(19) United States
(12) Patent Application Publication
(10) Pub. No.: US 2022/0387487 A1
(43) Pub. Date: Dec. 8, 2022

(54) COMPOSITIONS AND METHODS FOR TREATMENT OF CANCER

(71) Applicant: Aleta Biotherapeutics Inc., Natick, MA (US)

(72) Inventors: Roy Lobb, Wellesley, MA (US); Paul Rennert, Holliston, MA (US)

(21) Appl. No.: 17/605,711

(22) PCT Filed: Apr. 24, 2020
(86) PCT No.: PCT/US2020/029967

§ 371 Date: Oct. 22, 2021

Related U.S. Application Data
(60) Provisional application No. 62/839,376, filed on Apr. 26, 2019.

(51) Int. Cl. A61K 35/17; A61P 35/00

(52) U.S. Cl. CPC A61K 35/17; A61P 35/00

(57) ABSTRACT

Compositions, e.g., compositions comprising cellular therapeutics and/or protein therapeutics, and methods of using such compositions for treating cancer are described.

### CROSS REFERENCE TO RELATED APPLICATIONS

This application claims priority to U.S. Provisional Patent Application No. 62/839,376 the entire contents of which is hereby incorporated by reference.

### BACKGROUND

Adoptive cell therapy (ACT) is a treatment method in which cells are removed from a donor, cultured and/or manipulated in vitro, and then administered to a patient for the treatment of a disease. A variety of cell types have been used in ACT in an attempt to treat several classes of disorders. For the treatment of cancer, ACT generally involves the transfer of lymphocytes, such as chimeric antigen receptor (CAR) T cells. Use of such CAR T cells involves identifying an antigen on a tumor cell to which a CAR T cell can bind, but tumor heterogeneity can make antigen identification challenging. Accordingly, there remains a need for improved methods for treating cancer using adoptive cell therapy.

### SUMMARY

The present invention provides methods and compositions useful for treatment of cancer and/or for initiating or modulating immune responses. In some embodiments, the present invention provides cellular therapeutics (e.g., immune cells) comprising a constitutive expression construct, which comprises a promoter operably linked to a gene of interest. In some embodiments, the present invention provides cellular therapeutics (e.g., immune cells) comprising (i) an antigen binding receptor, wherein the antigen binding receptor comprises an antigen-binding domain, a transmembrane domain, and a cytosolic signaling domain, and (ii) an inducible expression construct, which comprises a promoter operably linked to a gene of interest. Among other things, the present invention encompasses the recognition that a combination of a cellular therapeutic described herein and one or more additional therapies (e.g., one or more additional cellular therapeutics (e.g., autologous CAR-T cell, allogenic CAR-T cell, CAR-NK cell, TCR-T cell, TIL cell, allogenic NK cell, autologous NK cell, gamma delta T cell, IPSC-derived cell, myeloid cell or other suitable cellular therapeutic cell type), antibody-drug conjugate, an antibody, and/or a polypeptide described herein), can lead to improved induction of beneficial immune responses, for example a cellular response (e.g., T-cell activation).

In some embodiments, the present disclosure provides methods of treating a subject having a tumor, comprising administering to the subject a cellular therapeutic described herein and/or a protein therapeutic described herein. In some embodiments, methods further comprise administration of one or more additional therapies (e.g., a second cellular therapeutic (e.g., autologous CAR-T cell, allogenic CAR-T cell, CAR-NK cell, TCR-T cell, TIL cell, allogenic NK cell, autologous NK cell, gamma delta T cell, IPSC-derived cell, myeloid cell or other suitable cellular therapeutic cell type), an antibody-drug conjugate, an antibody, and/or a polypeptide described herein).

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The figures of the drawing are for illustration purposes only, not for limitation.

FIG. 1 is a schematic depicting an exemplary cellular therapeutic.

FIG. 2 is a schematic depicting an exemplary “self amplifying” cellular therapeutic expressing an antigen binding receptor that does not include a signaling domain leading to induction of killing, and does include a signaling domain sufficient to induce gene transcription, and also encoding an inducible scFv-CD19 fusion protein and an inducible CAR (left) or a constitutively expressed CAR (right) that targets CD19.

FIG. 3 depicts sequence of CD19 variants from multi-mutant library. Amino acids at the diversified positions are indicated for numerous variants. The relative change in frequency from design to sorted population is presented in the lower table.

FIG. 4A is a ribbon diagram of the extracellular domain of CD19 highlighting potential regions for diversity to generate binding ligands. Orange: Ig domain 1 loops; blue: Ig domain 2 loops; red: Ig domain 2 sheet. FIG. 4B depicts tables of exemplary variant designs.

FIG. 5A depicts exemplary CD19 variants. Amino acids at the diversified positions are indicated for numerous clones. The relative change in frequency from design to sorted population is presented in the lower table.

FIG. 5B depicts exemplary CD19 variants. Amino acids at the diversified positions are indicated for numerous clones. The relative change in frequency from design to sorted population is presented in the lower table.

FIG. 5C depicts further exemplary CD19 variants. Amino acids at the diversified positions are indicated for numerous clones.

FIG. 5D depicts exemplary CD19 variants. Amino acids at the diversified positions are indicated for numerous clones.

FIG. 6 depicts exemplary CD19 variants. Amino acids at the diversified positions are indicated for numerous clones.

FIG. 7 depicts binding of biparatopic fusion proteins.

FIGS. 8A and 8B depicts cytotoxicity of a CD19-scFv protein.

FIG. 9 depicts cytotoxicity of purified biparatopic fusion proteins.

FIG. 10 depicts cytotoxicity of purified biparatopic fusion proteins.

FIG. 11 depicts cytotoxicity of purified biparatopic fusion proteins.

FIG. 12 depicts cytotoxicity of constitutively expressed biparatopic fusion proteins.

FIG. 13 depicts cytotoxicity of constitutively expressed biparatopic fusion proteins.

FIG. 14 depicts binding of purified biparatopic fusion proteins.

FIG. 15 depicts cytotoxicity of purified biparatopic fusion proteins.

FIG. 16A-16C demonstrate the equivalence of biparatopic fusion proteins with and without HIS tags.

FIG. 17 demonstrates the cytotoxicity equivalence of constitutively expressed biparatopic fusion proteins with and without HIS tags.

FIG. 18 demonstrates the cytotoxicity equivalence of constitutively expressed biparatopic fusion proteins with and without HIS tags in vivo.

FIG. 19 demonstrates the ability of biparatopic fusion proteins to bind multiple different alleles of CLEC12A.

FIG. 20 depicts binding of dual antigen fusion proteins.

FIGS. 21A and 21B depict cytotoxicity of dual antigen fusion proteins.

### DEFINITIONS

In order for the present invention to be more readily understood, certain terms are first defined below. Additional definitions for the following terms and other terms are set forth throughout the specification.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (including by intratracheal instillation), transdermal, vaginal and vitreal. In some embodiments, administration may be intratumoral or peritumoral. In some embodiments, administration may involve intermittent dosing. In some embodiments, administration may involve continuous dosing (e.g., perfusion) for at least a selected period of time.

Adoptive cell therapy: As used herein, “adoptive cell therapy” or “ACT” involves the transfer of immune cells with antitumour activity into cancer patients. In some embodiments, ACT is a treatment approach that involves the use of lymphocytes with antitumour activity, the in vitro expansion of these cells to large numbers and their infusion into a cancer-bearing host.

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Amelioration: As used herein, “amelioration” refers to prevention, reduction and/or palliation of a state, or improvement of the state of a subject. Amelioration includes, but does not require, complete recovery or complete prevention of a disease, disorder or condition.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are composed of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc regions of naturally-occurring antibodies bind to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered such glycosylation. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are fully human, or are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present disclosure is in a format selected from, but not limited to, intact IgG, IgE and IgM, bi- or multi-specific antibodies (e.g., Zybodies®, etc.), bi- or multi-paratopic antibodies, single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-Bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.)).

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antibody Fragment: As used herein, an “antibody fragment” includes a portion of an intact antibody, such as, for example, the antigen-binding or variable region of an antibody. Examples of antibody fragments include Fab, Fab′, F(ab′)₂, and Fv fragments; triabodies; tetrabodies; linear antibodies; single-chain antibody molecules; and multi-specific antibodies formed from antibody fragments. For example, antibody fragments include isolated fragments, “Fv” fragments (consisting of the variable regions of the heavy and light chains), recombinant single chain polypeptide molecules in which light and heavy chain variable regions are connected by a peptide linker (“scFv proteins”), recombinant single domain antibodies consisting of a variable region of an antibody heavy chain (e.g., VHH), and minimal recognition units consisting of the amino acid residues that mimic a hypervariable region (e.g., a hypervariable region of a heavy chain variable region (VH), a hypervariable region of a light chain variable region (VL), one or more CDR domains within the VH, and/or one or more CDR domains within the VL). In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)₂ fragment, scFv fragment, Fv fragment, dsFv diabody, dAb fragment, Fd′ fragment, Fd fragment, heavy chain variable region, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids.

Antigen: The term “antigen”, as used herein, refers to an agent that elicits an immune response; and/or an agent that binds to a T cell receptor (e.g., when presented by an MHC molecule) or to an antibody or antibody fragment. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer (e.g., other than a nucleic acid or amino acid polymer)) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source), or alternatively may exist on or in a cell. In some embodiments, an antigen is a recombinant antigen.

Antigen presenting cell: The phrase “antigen presenting cell” or “APC,” as used herein, has its art understood meaning referring to cells that process and present antigens to T-cells. Exemplary APC include dendritic cells, macrophages, B cells, certain activated epithelial cells, and other cell types capable of TCR stimulation and appropriate T cell costimulation.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 1%, 90%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

Cancer: The terms “cancer”, “malignancy”, “neoplasm”, “tumor”, and “carcinoma” are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias (T cell, B cell and myeloid), lymphomas (Hodgkins and non-Hodgkins), myelomas, myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Chimeric antigen receptor: “Chimeric antigen receptor” or “CAR” or “CARs” as used herein refers to engineered receptors, which graft an antigen specificity onto cells (for example T cells such as naive T cells, central memory T cells, effector memory T cells or combination thereof). CARs are also known as artificial T-cell receptors, chimeric T-cell receptors or chimeric immunoreceptors. In some embodiments, CARs comprise an antigen-specific targeting regions, an extracellular domain, a transmembrane domain, one or more co-stimulatory domains, and an intracellular signaling domain. In some embodiments, CARs comprise more than one antigen-specific targeting region, an extracellular domain, a transmembrane domain, one or more co-stimulatory domains, and an intracellular signaling domain. For example, CARs may have bispecific antigen targeting regions, whereby two distinct antigens are recognized.

Combination Therapy: As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

Domain: The term “domain” is used herein to refer to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecular (e.g., a small molecule, carbohydrate, a lipid, a nucleic acid, or a polypeptide). In some embodiments, a domain is a section of a polypeptide; in some such embodiments, a domain is characterized by a particular structural element (e.g., a particular amino acid sequence or sequence motif, α-helix character, β-sheet character, coiled-coil character, random coil character, etc.), and/or by a particular functional feature (e.g., binding activity, enzymatic activity, folding activity, signaling activity, etc.).

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Effector Function: As used herein, “effector function” refers a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP), and complement-mediated cytotoxicity (CMC). In some embodiments, an effector function is one that operates after the binding of an antigen, one that operates independent of antigen binding, or both.

Effector Cell: As used herein, “effector cell” refers to a cell of the immune system that expresses one or more Fc receptors and mediates one or more effector functions. In some embodiments, effector cells may include, but may not be limited to, one or more of monocytes, macrophages, neutrophils, dendritic cells, eosinophils, mast cells, platelets, large granular lymphocytes, Langerhans' cells, natural killer (NK) cells, T-lymphocytes, B-lymphocytes and may be from any organism including but not limited to humans, mice, rats, rabbits, and monkeys.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Extracellular domain: As used herein, “extracellular domain” (or “ECD”) refers to a portion of a polypeptide that extends beyond the transmembrane domain into extracellular space.

Fusion protein: As used herein, the term “fusion protein” generally refers to a polypeptide including at least two segments, each of which shows a high degree of amino acid identity to a peptide moiety that (1) occurs in nature, and/or (2) represents a functional domain of a polypeptide. Typically, a polypeptide containing at least two such segments is considered to be a fusion protein if the two segments are moieties that (1) are not included in nature in the same peptide, and/or (2) have not previously been linked to one another in a single polypeptide, and/or (3) have been linked to one another through action of the hand of man.

Gene: As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode functional RNA molecules such as tRNAs, RNAi-inducing agents, etc. For the purpose of clarity we note that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a protein-coding nucleic acid.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Immune response: As used herein, the term “immune response” refers to a response elicited in an animal. An immune response may refer to cellular immunity, humoral immunity or may involve both. An immune response may also be limited to a part of the immune system. For example, in certain embodiments, an immunogenic composition may induce an increased IFNγ response. In certain embodiments, an immunogenic composition may induce a mucosal IgA response (e.g., as measured in nasal and/or rectal washes). In certain embodiments, an immunogenic composition may induce a systemic IgG response (e.g., as measured in serum). In certain embodiments, an immunogenic composition may induce virus-neutralizing antibodies or a neutralizing antibody response. In certain embodiments, an immunogenic composition may induce a cytolytic (CTL) response by T cells.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein.

Individual, subject, patient: As used herein, the terms “subject,” “individual” or “patient” refer to a human or a non-human mammalian subject. The individual (also referred to as “patient” or “subject”) being treated is an individual (fetus, infant, child, adolescent, or adult) suffering from a disease, for example, cancer. In some embodiments, the subject is a human.

Linker: As used herein, the term “linker” refers to, e.g., in a fusion protein, an amino acid sequence of an appropriate length other than that appearing at a particular position in the natural protein and is generally designed to be flexible and/or to interpose a structure, such as an a-helix, between two protein moieties. In general, a linker allows two or more domains of a fusion protein to retain 50%, 55%, 60%, 65%, 70%, 75%, 80%, 85%, 90%, 95% or more of the biological activity of each of the domains. A linker may also be referred to as a spacer.

Masking moiety: As used herein, “masking moiety” refers to a molecular moiety that, when linked to an antigen-binding protein described herein, is capable of masking the binding of such antigen-binding moiety to its target antigen. An antigen-binding protein comprising such a masking moiety is referred to herein as a “masked” antigen-binding protein.

Nucleic acid. As used herein, “nucleic acid”, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to individual nucleic acid residues (e.g., nucleotides and/or nucleosides); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1,000, 1500, 2000, 2500, 3000, 3500, 4000, 4500, 5000 or more residues long. In some embodiments, a nucleic acid is single stranded; in some embodiments, a nucleic acid is double stranded. In some embodiments a nucleic acid has a nucleotide sequence comprising at least one element that encodes, or is the complement of a sequence that encodes, a polypeptide. In some embodiments, a nucleic acid has enzymatic activity.

Operably linked: As used herein, “operably linked” refers to a juxtaposition wherein the components described are in a relationship permitting them to function in their intended manner. A control sequence “operably linked” to one or more coding sequence(s) is ligated in such a way that expression of the one or more coding sequence(s) is achieved under conditions compatible with the control sequences. “Operably linked” sequences include both expression control sequences that are contiguous with the gene(s) of interest and expression control sequences that act in trans or at a distance to control the gene(s) of interest. The term “expression control sequence” as used herein refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

Paratope: As used herein, the term “paratope” refers to a portion of an antigen-binding polypeptide (e.g., antibody) that binds to an epitope of an antigen. As used herein, the term “biparatopic” (in the context of an antibody, construct or fusion protein described herein) refers to an antibody or construct that includes two paratopes, each of which binds to a different epitope on a single antigen. As used herein, the term “multiparatopic” (in the context of an antibody or a construct described herein) refers to an antibody or construct that includes two or more paratopes, each of which binds to a different epitope on a single antigen. In some embodiments, the two or more paratopes of a multiparatopic antibody or a fusion protein described herein bind to non-overlapping epitopes on a single antigen. In some embodiments, the two or more paratopes of a multiparatopic antibody or a fusion protein described herein bind to two epitopes on a single antigen that can share 1, 2, or 3 amino acids.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Peptide: The term “peptide” as used herein refers to a polypeptide that is typically relatively short, for example having a length of less than about 100 amino acids, less than about 50 amino acids, less than 20 amino acids, or less than 10 amino acids.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Promoter: As used herein, a “promoter” is a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide that encodes or specifies a gene product, causes the gene product to be produced in a cell under most or all physiological conditions of the cell. An “inducible” promoter is a nucleotide sequence that, when operably linked with a polynucleotide that encodes or specifies a gene product, causes the gene product to be produced in a cell substantially only when a promoter-specific inducer is present in the cell.

Protein: As used herein, the term “protein”, refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof.

Reference: As used herein, “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Solid tumor: As used herein, the term “solid tumor” refers to an abnormal mass of tissue that usually does not contain cysts or liquid areas. Solid tumors may be benign or malignant. Different types of solid tumors are named for the type of cells that form them. Examples of solid tumors are sarcomas, carcinomas, lymphomas, mesothelioma, neuroblastoma, retinoblastoma, etc.

Stage of cancer: As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

Subject: By “subject” is meant a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, or condition (e.g., cancer) has been diagnosed with and/or exhibits one or more symptoms of the disease, disorder, or condition.

Symptoms are reduced: According to the present invention, “symptoms are reduced” when one or more symptoms of a particular disease, disorder or condition is reduced in magnitude (e.g., intensity, severity, etc.) or frequency. For purposes of clarity, a delay in the onset of a particular symptom is considered one form of reducing the frequency of that symptom. It is not intended that the present invention be limited only to cases where the symptoms are eliminated. The present invention specifically contemplates treatment such that one or more symptoms is/are reduced (and the condition of the subject is thereby “improved”), albeit not completely eliminated.

T cell receptor: As used herein, a “T cell receptor” or “TCR” refers to the antigen-recognition molecules present on the surface of T-cells. During normal T-cell development, each of the four TCR genes, α, β, γ, and δ, can rearrange leading to highly diverse TCR proteins.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, “therapeutically effective amount” refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in one or more pro-angiogenic markers, an increase in anti-angiogenic markers, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen. In the context of cellular therapeutics a therapeutically effective dose can depend on diverse variables including the status of the patient cells used to create the cellular therapeutic, the dose of cells then administered to the patient, the expansion and persistence of those injected cells in the patient after injection and over time, the tumor antigen burden, the degree of pretreatment lymphodepletion and other factors known and unknown.

Transformation: As used herein, “transformation” refers to any process by which exogenous DNA or RNA is introduced into a host cell. Transformation may occur under natural or artificial conditions using various methods well known in the art. Transformation may rely on any known method for the insertion of foreign nucleic acid sequences into a prokaryotic or eukaryotic host cell. In some embodiments, a particular transformation methodology is selected based on the host cell being transformed and may include, but is not limited to, viral infection “transduction”), and transfection techniques, for example, electroporation, lipofection. In some embodiments, a “transformed” cell is stably transformed in that the inserted DNA is capable of replication either as an autonomously replicating plasmid or as part of the host chromosome. In some embodiments, a transformed cell transiently expresses introduced nucleic acid for limited periods of time. In some embodiments, a “transformed” cell is transformed in that the inserted DNA or RNA is a transposon or transposable element.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Tumor infiltrating lymphocyte: As used herein, the term “tumor-infiltrating lymphocytes” (TIL or TILs) refers to white blood cells of a subject afflicted with a cancer (such as melanoma), that have left the blood stream and have migrated into a tumor. In some embodiments, tumor-infiltrating lymphocytes have tumor specificity.

Vector: As used herein, “vector” refers to a nucleic acid molecule capable of transporting another nucleic acid to which it is associated. In some embodiments, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

### DETAILED DESCRIPTION

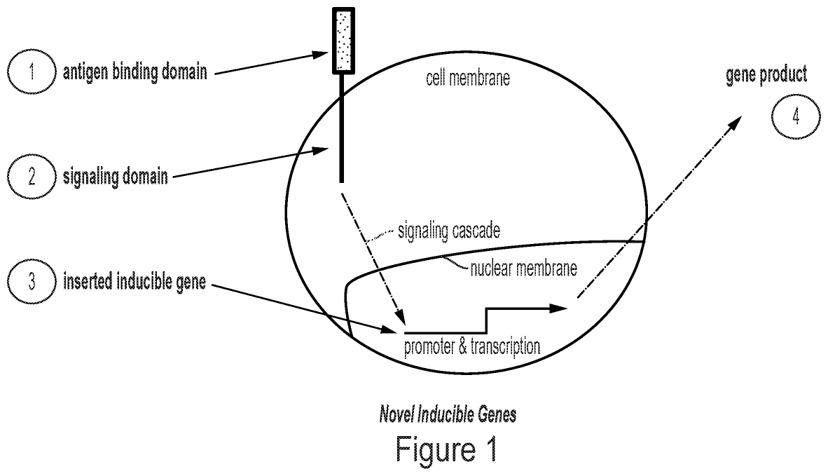

The disclosure is based, at least in part, on the discovery that a biparatopic fusion protein (e.g., a biparatopic fusion protein including an anti-CLL-1 scFv, an anti-CLL-1 VHH, and a CD19 variant) not only bound to CLL-1, but resulted in CAR-CD19 mediated cytotoxicity at a surprisingly greater level than that generated using a corresponding fusion protein that included an anti-CLL-1 scFv/CD19 variant or a corresponding fusion protein that included an anti-CLL-1 VHH/CD19 variant. Among other things, the present disclosure provides methods and compositions useful for treatment of cancer. Specifically, the present disclosure provides cellular therapeutics, e.g., immune cells, genetically modified with an integrated gene, e.g., a nucleotide sequence encoding one or more biparatopic fusion proteins described herein (e.g., a constitutive expression construct and/or an inducible expression construct that includes such nucleotide sequence). In some embodiments, expression of a nucleotide sequence encoding a biparatopic fusion protein described herein can be designed to be constitutive or inducible by appropriate selection, construction and/or design of an expressed promoter sequence operably linked to such nucleotide sequence, as described herein. In the case of a constitutive expression construct, a gene in the construct is constitutively expressed. In the case of an inducible expression construct, a cellular therapeutic can be genetically modified with a nucleic acid encoding an antigen binding receptor and with an inducible expression construct. Upon binding of a target antigen, an antigen binding receptor of a cellular therapeutic induces expression of a gene included in an inducible expression construct, e.g., as depicted in FIG. 1. In certain embodiments, expression of such gene facilitates and/or improves treatment of cancer, e.g., by one or more cellular therapies. The disclosure also specifically discloses protein therapeutics that include biparatopic fusion proteins encoded by such genes (e.g., soluble forms of such gene products, e.g., pharmaceutical compositions that include such proteins for administration), and nucleic acids encoding such biparatopic fusion proteins, such as for gene therapy.

### Constitutive Expression Constructs

In some embodiments, the disclosure includes constitutive expression constructs. In some embodiments, a constitutive expression construct comprises a nucleic acid sequence that includes at least a promoter operably linked to a nucleotide sequence encoding a biparatopic fusion protein described herein. A constitutive expression construct can comprise regulatory sequences, such as transcription and translation initiation and termination codons. In some embodiments, such regulatory sequences are specific to the type of cell into which the non-inducible expression construct is to be introduced, as appropriate. A constitutive expression construct can comprise a native or non-native promoter operably linked to a nucleotide sequence encoding a biparatopic fusion protein. Preferably, the promoter is functional in immune cells. Exemplary promoters include, e.g., CMV, E1F, VAV, TCRvbeta, MCSV, and PGK promoter. Operably linking of a nucleotide sequence with a promoter is within the skill of the artisan. In some embodiments, a constitutive expression construct is or includes a recombinant expression vector described herein.

### Inducible Expression Constructs and Inducible Expression

For inducible expression, a cellular therapeutic of the present disclosure can include (i) one or more types of antigen binding receptors comprising an extracellular domain, a transmembrane domain, and an intracellular (or cytoplasmic) domain, and (ii) an inducible expression construct.

Antigen Binding Receptors

The extracellular domain of an antigen binding receptor comprises a target-specific antigen binding domain. The intracellular domain (or cytoplasmic domain) of an antigen binding receptor comprises a signaling domain. The signaling domain includes an amino acid sequence that, upon binding of target antigen to the antigen binding domain, initiates and/or mediates an intracellular signaling pathway that can activate, among other things, an inducible expression construct described herein, such that an inducible gene is expressed. In some embodiments, a signaling domain further includes one or more additional signaling regions (e.g., costimulatory signaling regions) that activate one or more immune cell effector functions (e.g., native immune cell effector functions). In some embodiments, the signaling domain activates T cell activation, proliferation, survival, or other T cell function, but does not induce cytotoxic activity. In some embodiments, an antigen binding receptor includes all or part of a chimeric antigen receptor (CAR). Such CARs are known in the art (see, e.g., Gill et al., Immunol. Rev. 263:68-89 (2015); Stauss et al., Curr. Opin. Pharmacol. 24:113-118 (2015)).

Antigen Binding Domain

An antigen binding domain can be or include any polypeptide that specifically binds to a target antigen, e.g., a tumor antigen described herein. For example, in some embodiments, an antigen binding domain includes an antibody or antigen-binding fragment described herein (e.g., an Fab fragment, Fab′ fragment, F(ab′)₂ fragment, scFv fragment, Fv fragment, dsFv diabody, dAb fragment, Fd′ fragment, Fd fragment, an isolated complementarity determining region (CDR), a cameloid antibody, a masked antibody (e.g., Probody®), a single chain or Tandem diabody (TandAb®), a VHH, an Anticalin®, a single-domain antibody (e.g., Nanobody®), an ankyrin repeat protein or DARPIN®, an Avimer®, an Adnectin®, an Affilin®, an Affibody®, a Fynomer®, or a Centyrin®). In some embodiments, an antigen binding domain is or includes a T cell receptor (TCR) or antigen-binding portion thereof. In some embodiments, an antigen binding domain is a pH sensitive domain (see, e.g., Schroter et al., MAbs 7:138-51 (2015)). In some embodiments a polypeptide (e.g., CD19 or a CD19 variant described herein) can be engineered to function as an antigen binding domain.

Antigen binding domains can be selected based on, e.g., type and number of target antigens present on or near a surface of a target cell. For example, an antigen binding domain can be chosen to recognize an antigen that acts as a cell surface marker on a target cell associated with a particular disease state. In some embodiments, an antigen binding domain is selected to specifically bind to an antigen on a tumor cell. Tumor antigens are proteins that are produced by tumor cells and, in some embodiments, that elicit an immune response, for example T-cell mediated anti-tumor immune responses. Selection of an antigen binding domain can depend on, e.g., a particular type of cancer to be treated.

Transmembrane Domain

In general, a “transmembrane domain”, as used herein, refers to a domain having an attribute of being present in the membrane (e.g., spanning a portion or all of a cellular membrane). As will be appreciated, it is not required that every amino acid in a transmembrane domain be present in the membrane. For example, in some embodiments, a transmembrane domain is characterized in that a designated stretch or portion of a protein is substantially located in the membrane. As is well known in the art, amino acid or nucleic acid sequences may be analyzed using a variety of algorithms to predict protein subcellular localization (e.g., transmembrane localization). Exemplary such programs include psort (PSORT.org), Prosite (prosite.expasy.org), among others.

The type of transmembrane domain included in an antigen binding receptor described herein is not limited to any particular type. In some embodiments, a transmembrane domain is selected that is naturally associated with an antigen binding domain and/or intracellular domain. In some instances, a transmembrane domain includes a modification of one or more amino acids (e.g., deletion, insertion, and/or substitution), e.g., to avoid binding of such domains to a transmembrane domain of the same or different surface membrane proteins to minimize interactions with other members of the receptor complex.

A transmembrane domain can be derived either from a natural or from a synthetic source. Where the source is natural, a domain may be derived from any membrane-bound or transmembrane protein. Exemplary transmembrane regions can be derived from (e.g., can comprise at least a transmembrane region(s) of) an alpha, beta or zeta chain of a T-cell receptor, CD28, CD3 epsilon, CD45, CD4, CD5, CD8, CD9, CD16, CD22, CD27, CD33, CD37, CD64, CD80, CD86, CD134, CD137, TNFSFR25, or CD154. Alternatively, a transmembrane domain can be synthetic (and can, e.g., comprise predominantly hydrophobic residues such as leucine and valine). In some embodiments, a triplet of phenylalanine, tryptophan and valine are included at each end of a synthetic transmembrane domain. In some embodiments, a transmembrane domain is directly linked to a cytoplasmic domain. In some embodiments, a short oligo- or polypeptide linker (e.g., between 2 and 10 amino acids in length) may form a linkage between a transmembrane domain and an intracellular domain. In some embodiments, a linker is a glycine-serine doublet.

Cytoplasmic Domain

The intracellular domain (or cytoplasmic domain) comprises a signaling domain that, upon binding of target antigen to the antigen binding domain, initiates and/or mediates an intracellular signaling pathway that induces expression of an inducible expression construct described herein.

Intracellular signaling domains that can transduce a signal upon binding of an antigen to an immune cell are known, any of which can be used herein. For example, cytoplasmic sequences of a T cell receptor (TCR) are known to initiate signal transduction following TCR binding to an antigen (see, e.g., Brownlie et al., Nature Rev. Immunol. 13:257-269 (2013)). In some embodiments, a signaling domain includes an immunoreceptor tyrosine-based activation motif (ITAM). Examples of ITAM containing cytoplasmic signaling sequences include those derived from TCR zeta, FcR gamma, FcR beta, CD3 zeta, CD3 gamma, CD3 delta, CD3 epsilon, CD5, CD22, CD79a, CD79b, and CD66d (see, e.g., Love et al., Cold Spring Harb. Perspect. Biol. 2:a002485 (2010); Smith-Garvin et al., Annu. Rev. Immunol. 27:591-619 (2009)).

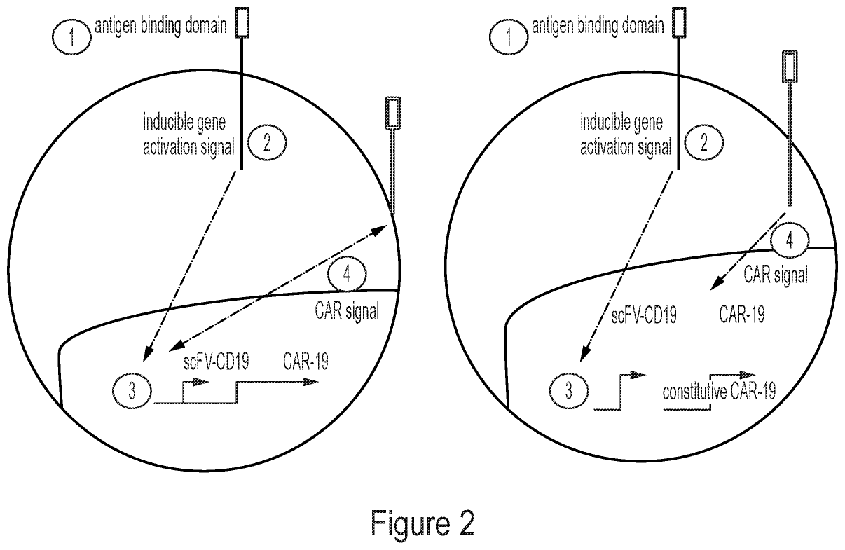

In some embodiments, an intracellular signaling domain does not include a sequence that transduces a signal leading to killing by T cells (e.g., CD8⁺ T cells). For example, TCR cytoplasmic sequences are known to activate a number of signaling pathways, some of which lead to killing (see, e.g., Smith-Garvin et al., Annu. Rev. Immunol. 27:591-619 (2009)). In some embodiments, an intracellular domain includes a signaling domain that leads to signal transduction that mediates expression of an inducible expression construct, but not induction of killing (e.g., as exemplified in FIG. 2). For example, the cytoplasmic domain can include a cytoplasmic portion of a PDGF receptor and, upon antigen binding by the antigen binding domain, can lead to an intracellular signal that induces a promoter of the inducible expression construct. One of skill in the art, based on knowledge in the art, can select an intracellular domain and a cognate promoter to be included within an inducible expression construct.

It is known that signals generated through a TCR alone are insufficient for full activation of a T cell and that a secondary or co-stimulatory signal is also required. Thus, in some embodiments, a signaling domain further includes one or more additional signaling regions (e.g., costimulatory signaling regions) that activate one or more immune cell effector functions (e.g., a native immune cell effector function described herein). In some embodiments, a portion of such costimulatory signaling regions can be used, as long as the portion transduces the effector function signal. In some embodiments, a cytoplasmic domain described herein includes one or more cytoplasmic sequences of a T cell co-receptor (or fragment thereof). Non-limiting examples of such T cell co-receptors include CD27, CD28, 4-1BB (CD137), OX40, CD30, CD40, PD-1, ICOS, lymphocyte function-associated antigen-1 (LFA-1), MYD88, CD2, CD7, LIGHT, NKG2C, B7-H₃, and a ligand that specifically binds with CD83.

In some embodiments, two or more signaling domains are linked to each other in a random or specified order. Optionally, a short oligo- or polypeptide linker, (e.g., between 2 and 10 amino acids in length) may form the linkage. In some embodiments, such linker is a glycine-serine doublet.

Inducible Expression Constructs

In some embodiments, an “inducible expression construct” as used herein may be or comprises a nucleic acid sequence that includes at least a promoter operably linked to a nucleotide sequence encoding a biparatopic fusion protein described herein. An inducible expression construct can comprise regulatory sequences, such as transcription and translation initiation and termination codons. In some embodiments, such regulatory sequences are specific to the type of cell into which an inducible expression construct is to be introduced, as appropriate. In some embodiments, such regulatory sequences are specific to a signaling pathway induced by a signaling domain described herein.

An inducible expression construct can comprise a native or non-native promoter operably linked to the nucleic acid encoding a biparatopic fusion protein. Preferably, the promoter is functional in immune cells. Operably linking of a nucleotide sequence with a promoter is within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus. In some embodiments, a promoter includes an NFAT, NF-κB, AP-1 or other recognition sequence, as examples.

In some embodiments, a promoter included in an inducible expression construct described herein is an IL-2 promoter, a cell surface protein promoter (e.g., CD69 promoter), a cytokine promoter (e.g., TNF promoter), a cellular activation promoter (e.g., CTLA4, OX40, CD40L), or a cell surface adhesion protein promoter (e.g., VLA-1 promoter). The selection of a promoter, e.g., strong, weak, inducible, tissue-specific, developmental-specific, having specific kinetics of activation (e.g., early and/or late activation), and/or having specific kinetics of expression of an induced gene (e.g., short or long expression) is within the ordinary skill of the artisan. In some embodiments, a promoter mediates rapid, sustained expression, measured in days (e.g., CD69). In some embodiments, a promoter mediates delayed expression, termed late-inducible (e.g., VLA1). In some embodiments, a promoter mediates rapid, transient expression (e.g., TNF, immediate early response genes and many others).

Upon antigen binding by an antigen binding receptor, a signal can be transduced from a signaling domain of an antigen binding receptor described herein to an inducible expression construct, e.g., using a known pathway (see, e.g., Chow et al., Mol. Cell. Biol. 19:2300-2307 (1999); Castellanos et al., J. Immunol. 159:5463-73 (1997); Kramer et al., JBC 270:6577-6583 (1995); Gibson et al., J. Immunol. 179:3831-40 (2007)); Tsytsykova et al., J. Biol. Chem. 271:3763-70 (1996); Goldstein et al., J. Immunol. 178:201-10 (2007)). Thus, upon binding of an antigen, an antigen binding receptor activates a signal transduction pathway that leads to induction of expression of a biparatopic fusion protein (e.g., by binding of a transcription factor to a promoter described herein).

### Biparatopic Fusion Proteins

In some embodiments, a cellular therapeutic described herein can include an expression construct (e.g., a constitutive expression construct or inducible expression construct) that encodes a biparatopic fusion protein. In some embodiments, a biparatopic fusion protein comprises two or more antigen binding proteins (e.g., antibodies or antibody fragments, e.g., Fab fragment, Fab′ fragment, F(ab′)₂ fragment, scFv fragment, Fv fragment, dsFv diabody, dAb fragment, Fd′ fragment, Fd fragment, CDR region, a cameloid antibody, a masked antibody (e.g., Probody®), a single chain or Tandem diabody (TandAb®), a VHH, an Anticalin®, a single-domain antibody (e.g., Nanobody®), an ankyrin repeat protein or DARPIN®, an Avimer®, an Adnectin®, an Affilin®, an Affibody®, a Fynomer®, or a Centyrin®) and at least one polypeptide antigen. In some embodiments, the two or more antigen binding proteins bind different epitopes of the same antigen. In some embodiments, the two or more antigen binding proteins bind to a tumor antigen (e.g., a TAA or TSA) as described herein. In some embodiments, a biparatopic fusion protein is or includes two antibody fragments and at least one additional non-antibody polypeptide. In some embodiments, the two or more antigen binding proteins include two or more antigen binding proteins that bind to a first tumor antigen and at least one antigen binding protein that binds a second tumor antigen. In some embodiments, a biparatopic fusion protein is or includes an scFv, a VHH, and at least one polypeptide antigen (e.g., CD19 or a CD19 variant described herein).

The two or more antigen binding proteins and at least one polypeptide antigen can be configured in any order within a biparatopic fusion protein. In some embodiments, the polypeptide antigen is linked (e.g., fused) to the amino terminus of one of the two or more antigen binding proteins. In some embodiments, the polypeptide antigen is linked (e.g., fused) to the carboxyl terminus of one of the two or more antigen binding proteins. For example, a biparatopic fusion protein that includes antigen binding protein A; antigen binding protein B; and a polypeptide antigen can be configured in any of the following configurations: (i) antigen binding protein A-antigen binding protein B-polypeptide antigen; (ii) antigen binding protein B-antigen binding protein A-polypeptide antigen; (iii) polypeptide antigen-antigen binding protein A-antigen binding protein B; (iv) polypeptide antigen-antigen binding protein B-antigen binding protein A; (v) antigen binding protein B-polypeptide antigen-antigen binding protein A; (vi) antigen binding protein A-polypeptide antigen-antigen binding protein B. For example, in a biparatopic fusion protein comprising an scFv (e.g., an scFv described herein), a VHH (e.g., a VHH described herein), and a polypeptide antigen (e.g., a CD19 variant described herein), the biparatopic fusion protein could be configured in any of the following configurations: (i) scFv-VHH—polypeptide antigen; (ii) scFv-polypeptide antigen—VHH; (iii) VHH-scFv—polypeptide antigen; (iv) VHH-polypeptide antigen—scFv; (v) polypeptide antigen—scFv-VHH; (vi) polypeptide antigen—VHH-scFv.

### Polypeptide Antigen

In some embodiments, a biparatopic fusion protein (or a fragment thereof) is expressed on the surface of the cellular therapeutic and/or is secreted by the cellular therapeutic and/or binds to the surface of a tumor cell. While any polypeptide antigen can be expressed from an expression construct described herein, in particular embodiments, a polypeptide antigen included in a biparatopic fusion protein is selected that is a target of (e.g., binds to) an antigen-binding protein described herein (e.g., an antibody or fragment thereof), an antibody fusion protein or an antibody-drug conjugate). In some embodiments, the antibody or antibody fusion protein can be, e.g., a known therapeutic antibody (e.g., one that exhibits ADCC or CDC), a therapeutic fusion protein, or a therapeutic antibody-drug conjugate. In some embodiments, a polypeptide antigen included in a biparatopic fusion protein is selected that is a target of (e.g., binds to) a CAR-bearing cell (e.g., a CAR-T cell).

In some embodiments, a polypeptide antigen binds one or more known anti-tumor antibodies. Various review articles have been published that describe useful anti-tumor antibodies (see, for example, Adler et al., Hematol. Oncol. Clin. North Am. 26:447-81 (2012); Li et al., Drug Discov. Ther. 7:178-84 (2013); Scott et al., Cancer Immun. 12:14 (2012); and Sliwkowski et al., Science 341:1192-1198 (2013)). Table 1 presents a non-comprehensive list of certain human polypeptide antigens targeted by known, available antibody agents, and notes certain cancer indications for which the antibody agents have been proposed to be useful:

TABLE 1

| | Antibody (commercial | |
|---|---|---|
| Human Antigen | or scientific name) | Cancer indication |
| CD2 | Siplizumab | Non-Hodgkin’s Lymphoma |
| CD3 | UCHT1 | |
| CD4 | HuMax-CD4 | Peripheral or Cutaneous T-cell |
| | | Lymphoma |
| CD19 | SAR3419, MEDI-551, | Diffuse Large B-cell Lymphoma |
| | FMC63 | |
| CD19 and CD3 or | Bispecific antibodies such | Non-Hodgkin’s Lymphoma |
| CD22 | as Blinatumomab, | |
| | DT2219ARL | |
| CD20 | Rituximab, Veltuzumab, | B cell malignancies (Non-Hodgkin's |
| | Tositumomab, | lymphoma, Chronic lymphocytic |
| | Ofatumumab, Ibritumomab, | leukemia) |
| | Obinutuzumab, | |
| CD22 (SIGLEC2) | Inotuzumab, | Chemotherapy-resistant hairy cell |
| | tetraxetan, CAT-8015, | leukemia, Hodgkin’s lymphoma |
| | DCDT2980S, Bectumomab | |
| CD30 | Brentuximab vedotin | |
| CD33 | Gemtuzumab ozogamicin | Acute myeloid leukemia |
| | (Mylotarg) | |
| CD37 | TRU-016 | Chronic lymphocytic leukemia |
| CD38 | Daratumumab | Multiple myeloma, hematological |
| | | tumors |
| CD40 | Lucatumumab | Non-Hodgkin’s lymphoma |
| CD52 | Alemtuzumab (Campath) | Chronic lymphocytic leukemia |
| CD56 (NCAM1) | Lorvotuzumab | Small Cell Lung Cancer |
| CD66e (CEA) | Labetuzumab | Breast, colon and lung tumors |
| CD70 | SGN-75 | Non-Hodgkin’s lymphoma |
| CD74 | Milatuzumab | Non-Hodgkin’s lymphoma |
| CD138 (SYND1) | BT062 | Multiple Myeloma |
| CD152 (CTLA-4) | Ipilimumab | Metastatic melanoma |
| CD221 (IGF1R) | AVE1642, IMC-A12, MK- | Glioma, lung, breast, head and |
| | 0646, R150, CP 751871 | neck, prostate and thyroid cancer |
| CD254 (RANKL) | Denosumab | Breast and prostate carcinoma |
| CD261 (TRAILR1) | Mapatumumab | Colon, lung and pancreas tumors |
| CD262 (TRAILR2) | HGS-ETR2, CS-1008 | and haematological malignancies |
| CD326 (Epcam) | Edrecolomab, 17-1A, | Colon and rectal cancer, malignant |
| | IGN101, Catumaxomab, | ascites, epithelial tumors (breast, |
| | Adecatumumab | colon, lung) |
| CD309 (VEGFR2) | IM-2C6, CDP791 | Epithelium-derived solid tumors |
| CD319 (SLAMF7) | HuLuc63 | Multiple myeloma |
| CD340 (HER2) | Trastuzumab, Pertuzumab, | Breast cancer |
| | Ado-trastuzumab | |
| | emtansine | |
| CAIX (CA9) | cG250 | Renal cell carcinoma |
| EGFR (c-erbB) | Cetuximab, Panitumumab, | Solid tumors including glioma, lung, |
| | nimotuzumab and 806 | breast, colon, and head and neck |
| | | tumors |
| EPHA3 (HEK) | KB004, IIIA4 | Lung, kidney and colon tumors, |
| | | melanoma, glioma and |
| | | haematological malignancies |
| Episialin | Epitumomab | Epithelial ovarian tumors |
| FAP | Sibrotuzumab and F19 | Colon, breast, lung, pancreas, and |
| | | head and neck tumors |
| HLA-DR beta | Apolizumab | Chronic lymphocytic leukemia, non- |
| | | Hodkin’s lymphoma |
| FOLR-1 | Farletuzumab | Ovarian tumors |
| 5T4 | Anatumomab | Non-small cell lung cancer |
| GD3/GD2 | 3F8, ch14.18, KW-2871 | Neuroectodermal and epithelial |
| | | tumors |
| gpA33 | huA33 | Colorectal carcinoma |
| GPNMB | Glembatumumab | Breast cancer |
| HER3 (ERBB3) | MM-121 | Breast, colon, lung, ovarian, and |
| | | prostate tumors |
| Integrin αVβ3 | Etaracizumab | Tumor vasculature |
| Integrin α5β1 | Volociximab | Tumor vasculature |
| Lewis-Y antigen | hu3S193, IgN311 | Breast, colon, lung and prostate |
| | | tumors |
| MET (HGFR) | AMG 102, METMAB, | Breast, ovary and lung tumors |
| | SCH900105 | |
| Mucin-1/CanAg | Pemtumomab, | Breast, colon, lung and ovarian |
| | oregovomab, Cantuzumab | tumors |
| PSMA | ADC, J591 | Prostate Cancer |
| Phosphatidylserine | Bavituximab | Solid tumors |
| TAG-72 | Minretumomab | Breast, colon and lung tumors |
| Tenascin | 81C6 | Glioma, breast and prostate |
| | | tumours |
| VEGF | Bevacizumab | Tumour vasculature |

In some embodiments, a biparatopic fusion protein described herein (or an expression construct (e.g., a constitutive expression construct or inducible expression construct) encoding one or more such polypeptide antigens), or a cellular therapeutic comprising such expression construct, is administered to a subject in combination with one or more of these (or other) known antibodies, or a fragment thereof. In some embodiments, a biparatopic fusion protein described herein (or an expression construct (e.g., a constitutive expression construct or inducible expression construct) encoding one or more such polypeptide antigens), or a cellular therapeutic comprising such expression construct, is administered to a subject in combination with a cellular therapeutic (e.g., a CAR-T cell) expressing one or more of these (or other) known antibodies, or a fragment thereof.

In some embodiments, a polypeptide antigen that binds to one or more known antibody-drug conjugates can be included in a biparatopic fusion protein described herein. Antibody-drug conjugates are known and include, e.g., brentuximab vedotin (ADCETRIS®, Seattle Genetics); ado-trastuzumab emtansine (KADCYLA®, Roche); Gemtuzumab ozogamicin (Wyeth); CMC-544; SAR3419; CDX-011; PSMA-ADC; BT-062; and IMGN901 (see, e.g., Sassoon et al., Methods Mol. Biol. 1045:1-27 (2013); Bouchard et al., Bioorganic Med. Chem. Lett. 24: 5357-5363 (2014)). In some such embodiments, a biparatopic fusion protein described herein (or an expression construct (e.g., a constitutive expression construct or inducible expression construct) encoding one or more such polypeptide antigens), or a cellular therapeutic comprising such expression construct, is administered to a subject in combination with one or more of these (or other) known antibody-drug conjugates.

CD19

In some embodiments the polypeptide antigen include in a biparatopic fusion protein is a tumor antigen. In some embodiments, the tumor antigen comprises CD19 or a fragment thereof. In some embodiments, the tumor antigen comprises a CD19 variant or a fragment thereof. In some embodiments, the tumor antigen comprises the extracellular domain (ECD), or fragment thereof of CD19 or a CD19 variant. In some embodiments, the tumor antigen comprises an epitope recognized by FMC63 (Nadler, Lee M “B Cell/Leukemia Panel Workshop: Summary and Comments” Leukocyte Typing II Ed. E. L. Reinherz et al., New York, 1986; Zola et al., Immunol Cell Biol. 69: 411-422; Nicholson et al., Mol Immunol. 34: 1157-1165).

CD19 is a 95 kDa type I transmembrane glycoprotein that is used as a biomarker of B cell development (Wang et al., Exp. Hematol. Oncol. 1:36 (2012)). CD19 expression in lymphoma and leukemia has made it an effective therapeutic target, especially for chimeric antigen receptor (CAR) T cell therapy (Maude et al., Blood 125:4017-4024 (2015)). Based on CD19's uniquely efficacious performance in CAR-T cell therapy, therapeutic approaches have been described that involve “converting” CD19⁻ tumors into CD19⁺ tumors using antibody-CD19 fusions or CD19 variants engineered to bind directly to tumor biomarkers (see, e.g., WO2017/075537 and WO2017/075533). In these contexts, the structural integrity—including proper folding, presentation of biological epitopes, and stability—of the CD19 extracellular region may be important to performance of the molecular therapy.

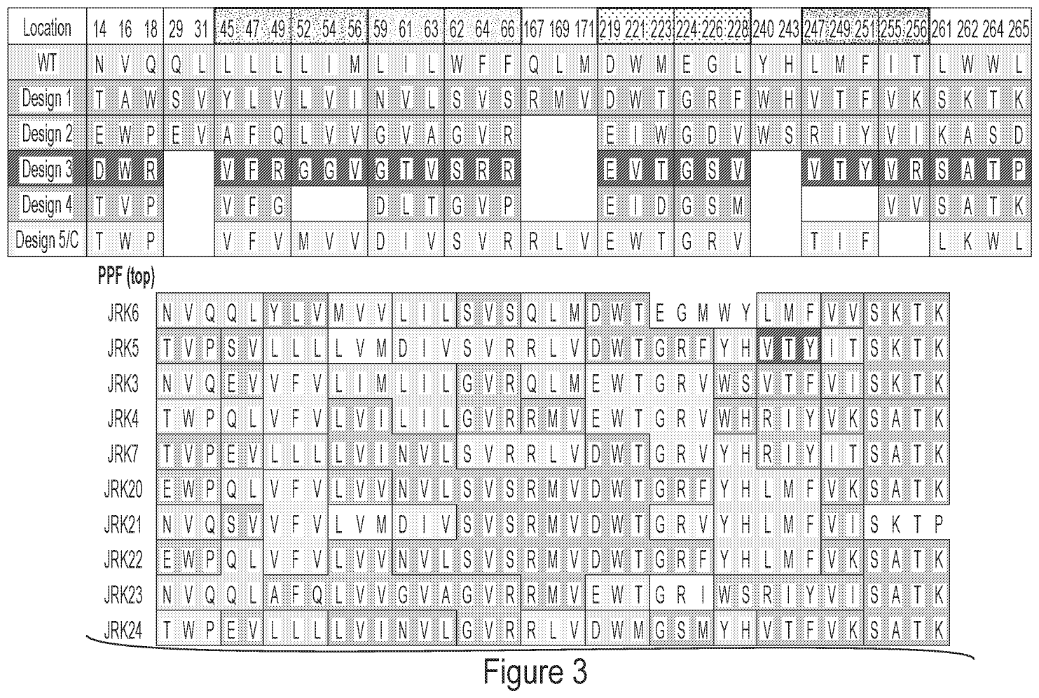

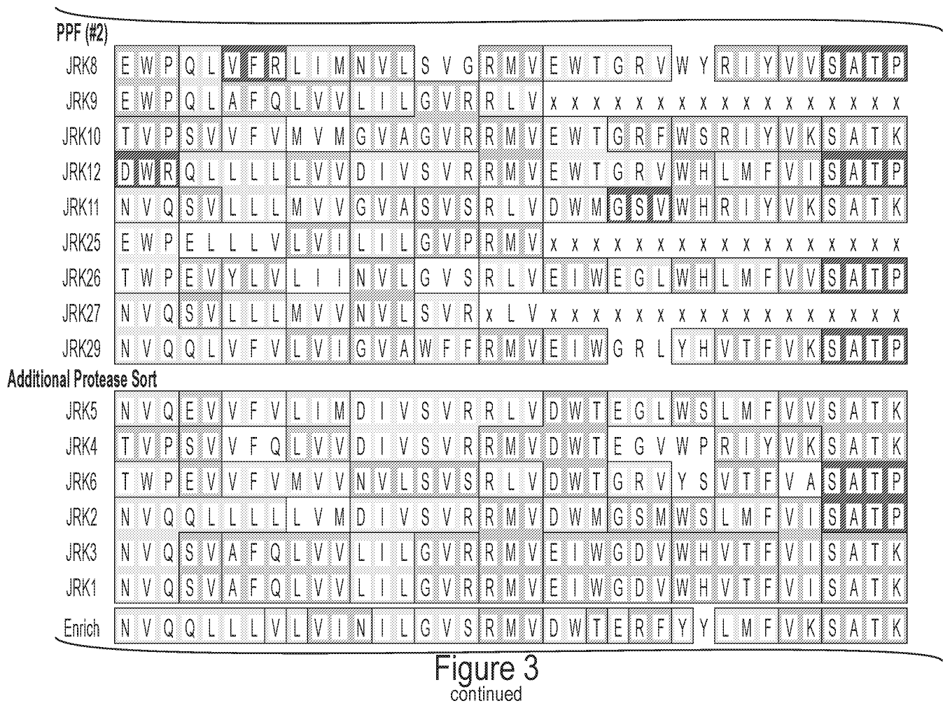

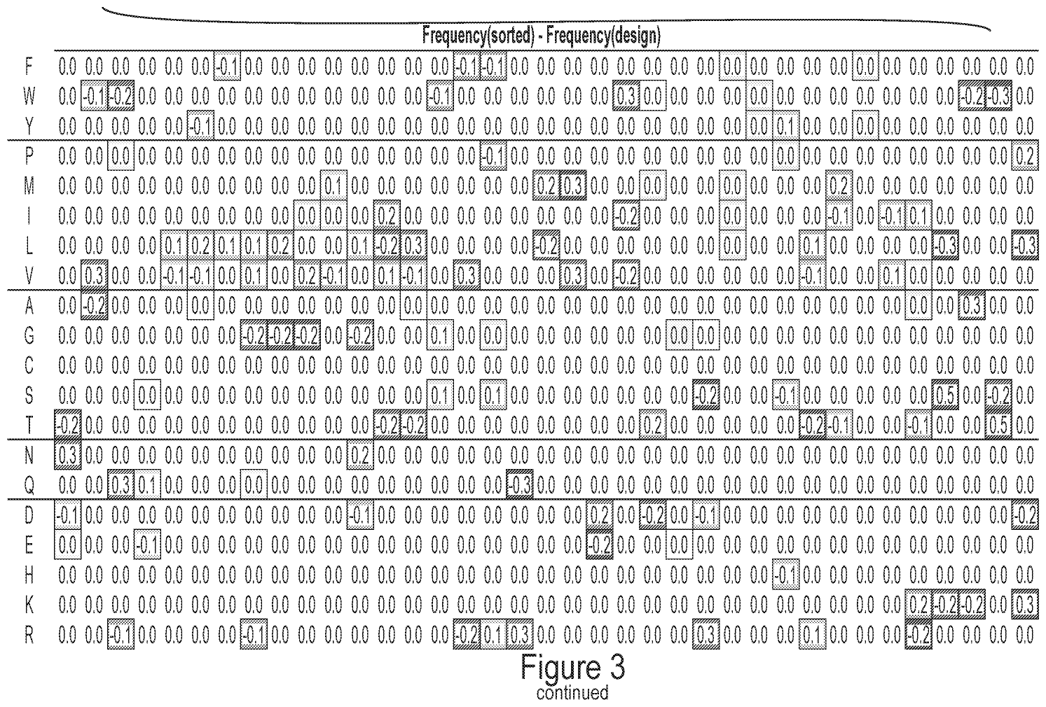

The extracellular region of CD19 was hypothesized to contain two C2-like immunoglobulin domains (see, e.g., Wang et al., Exp. Hematol. Oncol. 1:36 (2012); Tedder et al., Nat. Rev. Rheumatol. 5:572-577 (2009)). This is supported by homology modeling (Soding et al., Nucleic Acids Res. 33:244-248 (2005)) (see FIG. 3). However, a recently published structure demonstrated that CD19 does not include C2-like immunoglobulin domains (Teplyakov et al., Proteins 86:495-500 (2018)).

The nucleotide sequence of human CD19, as well as nucleotide sequences of specific domains of CD19, are known (see Genbank Accession No. M84371.1). For example, the nucleotide sequence encoding the extracellular domain of CD19 is:

(SEQ ID NO: 1)
CCCGAGGAACCTCTAGTGGTGAAGGTGGAAGAGGG
AGATAACGCTGTGCTGCAGTGCCTCAAGGGGACCT
CAGATGGCCCCACTCAGCAGCTGACCTGGTCTCGG
GAGTCCCCGCTTAAACCCTTCTTAAAACTCAGCCT
GGGGCTGCCAGGCCTGGGAATCCACATGAGGCCCC
TGGCCATCTGGCTTTTCATCTTCAACGTCTCTCAA
CAGATGGGGGGCTTCTACCTGTGCCAGCCGGGGCC
CCCCTCTGAGAAGGCCTGGCAGCCTGGCTGGACAG
TCAATGTGGAGGGCAGCGGGGAGCTGTTCCGGTGG
AATGTTTCGGACCTAGGTGGCCTGGGCTGTGGCCT
GAAGAACAGGTCCTCAGAGGGCCCCAGCTCCCCTT
CCGGGAAGCTCATGAGCCCCAAGCTGTATGTGTGG
GCCAAAGACCGCCCTGAGATCTGGGAGGGAGAGCC
TCCGTGTCTCCCACCGAGGGACAGCCTGAACCAGA
GCCTCAGCCAGGACCTCACCATGGCCCCTGGCTCC
ACACTCTGGCTGTCCTGTGGGGTACCCCCTGACTC
TGTGTCCAGGGGCCCCCTCTCCTGGACCCATGTGC
ACCCCAAGGGGCCTAAGTCATTGCTGAGCCTAGAG
CTGAAGGACGATCGCCCGGCCAGAGATATGTGGGT
AATGGAGACGGGTCTGTTGTTGCCCCGGGCCACAG
CTCAAGACGCTGGAAAGTATTATTGTCACCGTGGC
AACCTGACCATGTCATTCCACCTGGAGATCACTGC
TCGGCCAGTACTATGGCACTGGCTGCTGAGGACTG
GTGGCTGGAAG.

The amino acid sequence of the extracellular domain of CD19 is:

(SEQ ID NO: 2)
PEEPLVVKVEEGDNAVLQCLKGTSDGPTQQLTWSR
ESPLKPFLKLSLGLPGLGIHMRPLAIWLFIFNVSQ
QMGGFYLCQPGPPSEKAWQPGWTVNVEGSGELFRW
NVSDLGGLGCGLKNRSSEGPSSPSGKLMSPKLYVW
AKDRPEIWEGEPPCLPPRDSLNQSLSQDLTMAPGS
TLWLSCGVPPDSVSRGPLSWTHVHPKGPKSLLSLE
LKDDRPARDMWVMETGLLLPRATAQDAGKYYCHRG
NLTMSFHLEITARPVLWHWLLRTGGWK.

In some embodiments, a biparatopic fusion protein described herein comprises one or more CD19 variants. In some embodiments, a CD19 variant is or includes a full length CD19 polypeptide, or a portion thereof, that includes one or more amino acid substitutions described herein. In some embodiments, a CD19 variant is or includes a CD19 extracellular domain, or a portion thereof, that includes one or more amino acid substitutions described herein. In some embodiments, a CD19 variant is or includes a CD19 extracellular domain lacking 1, 2, 3, 4, 5, 6, 7, 8, 9, 10, 11, 12, 13, 14, 15, or more amino acids at the C-terminus, and includes one or more amino acid substitutions described herein.

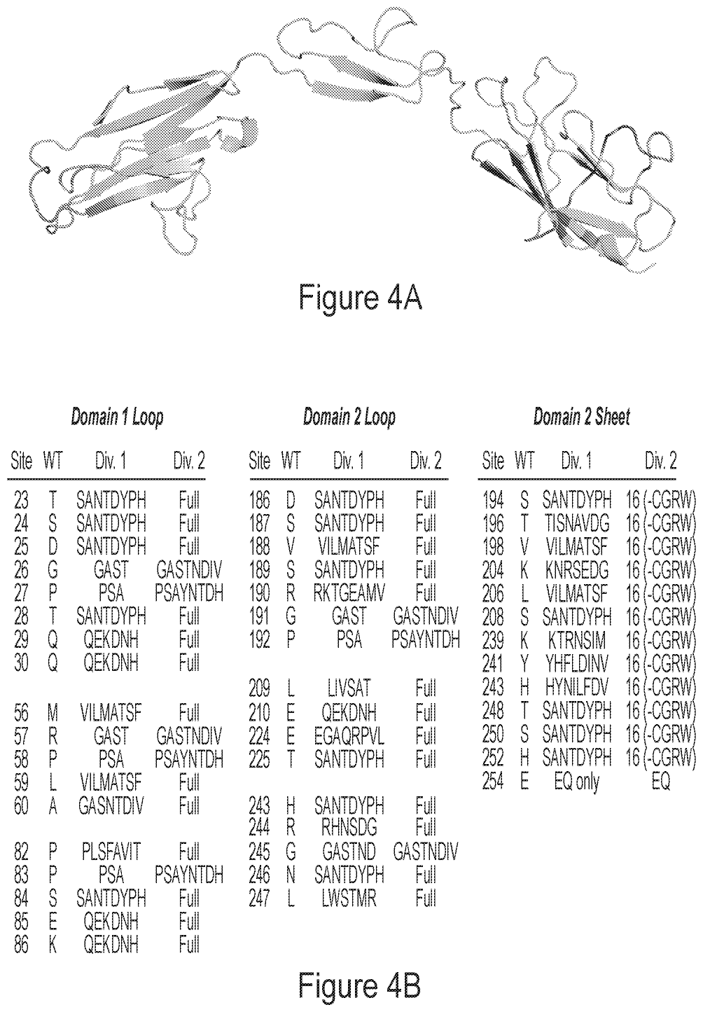

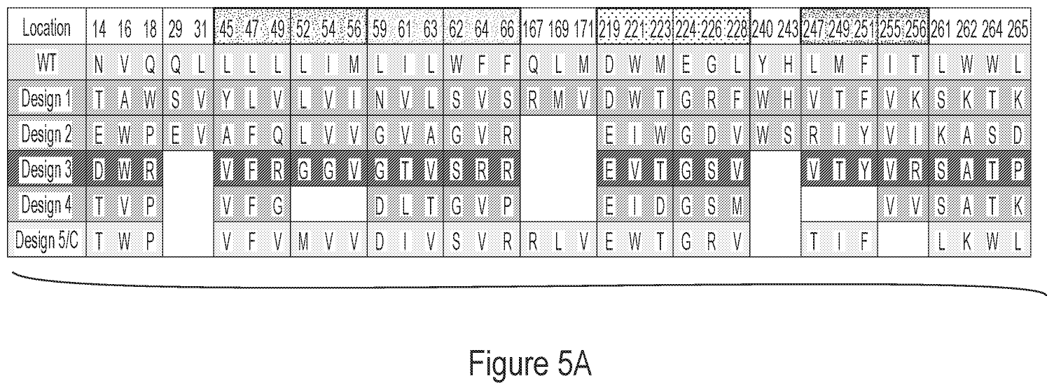

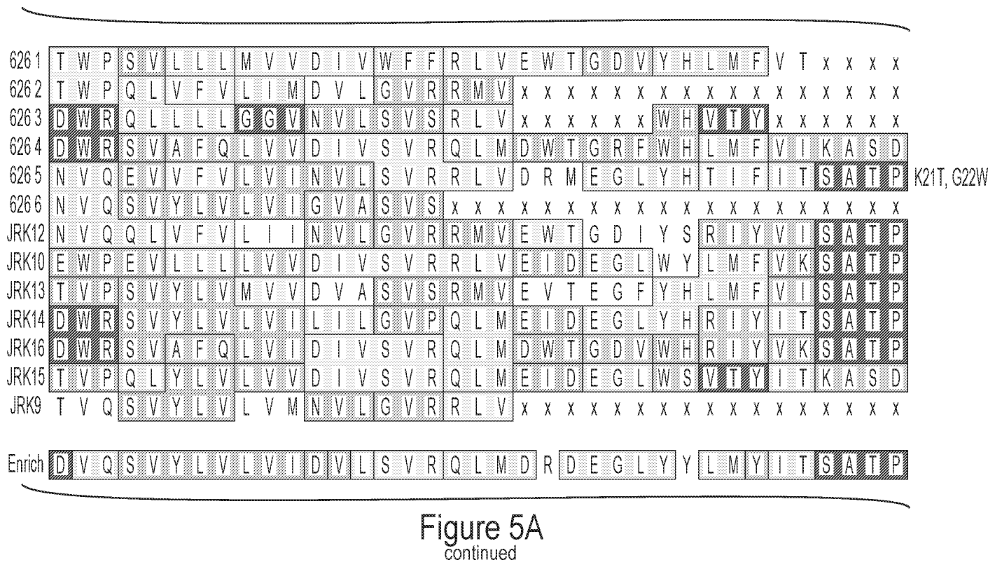

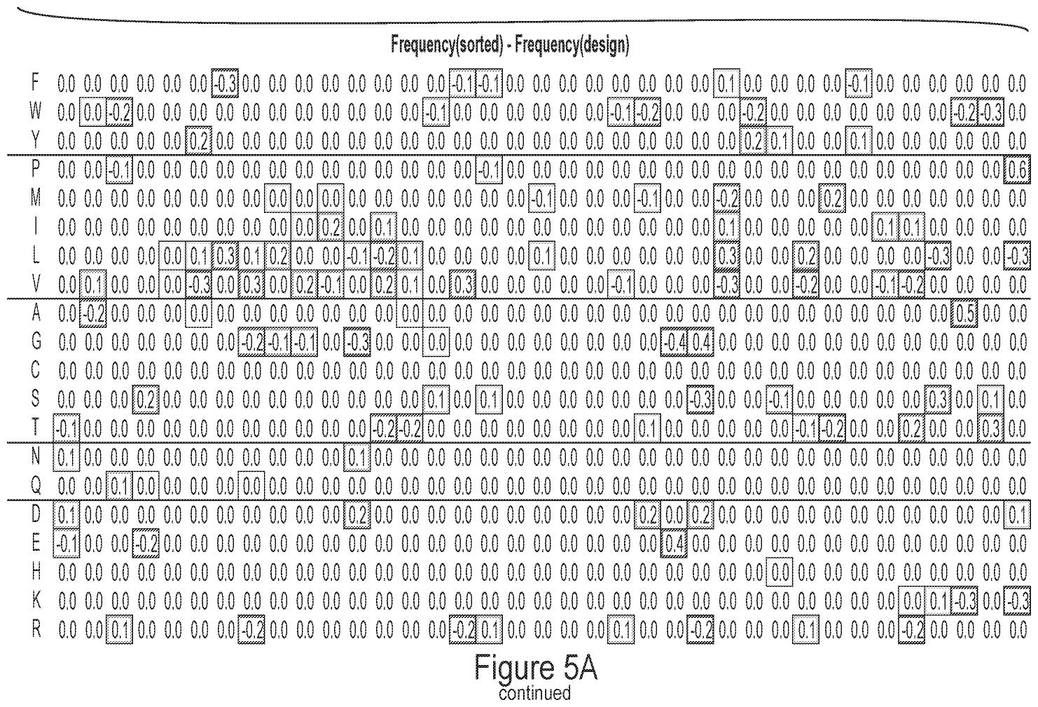

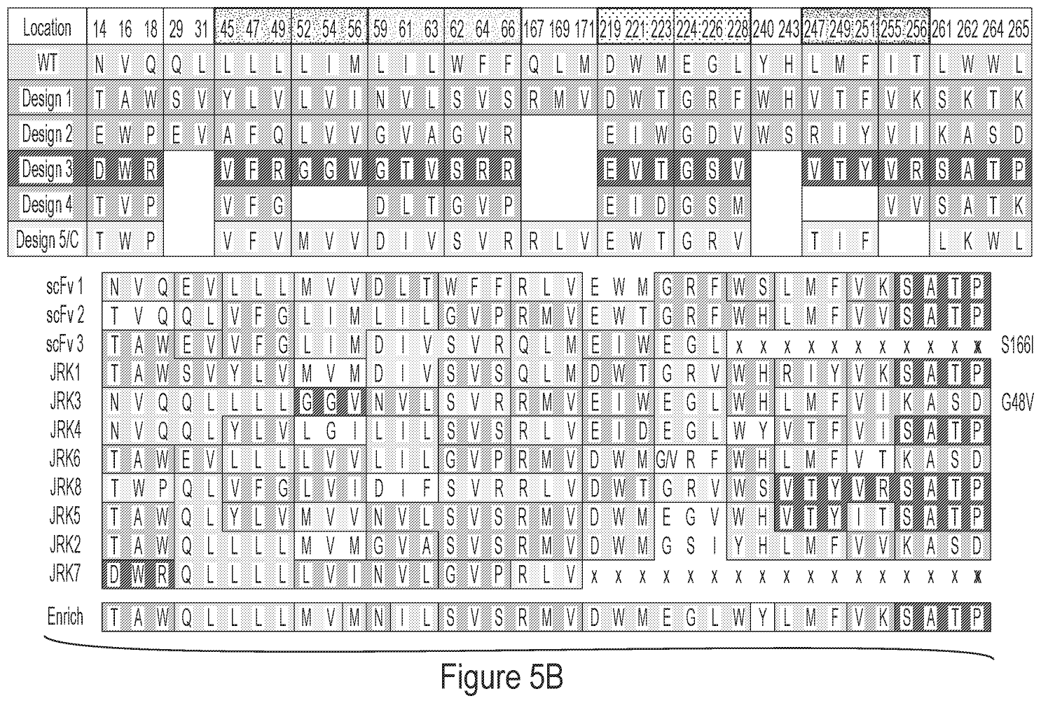

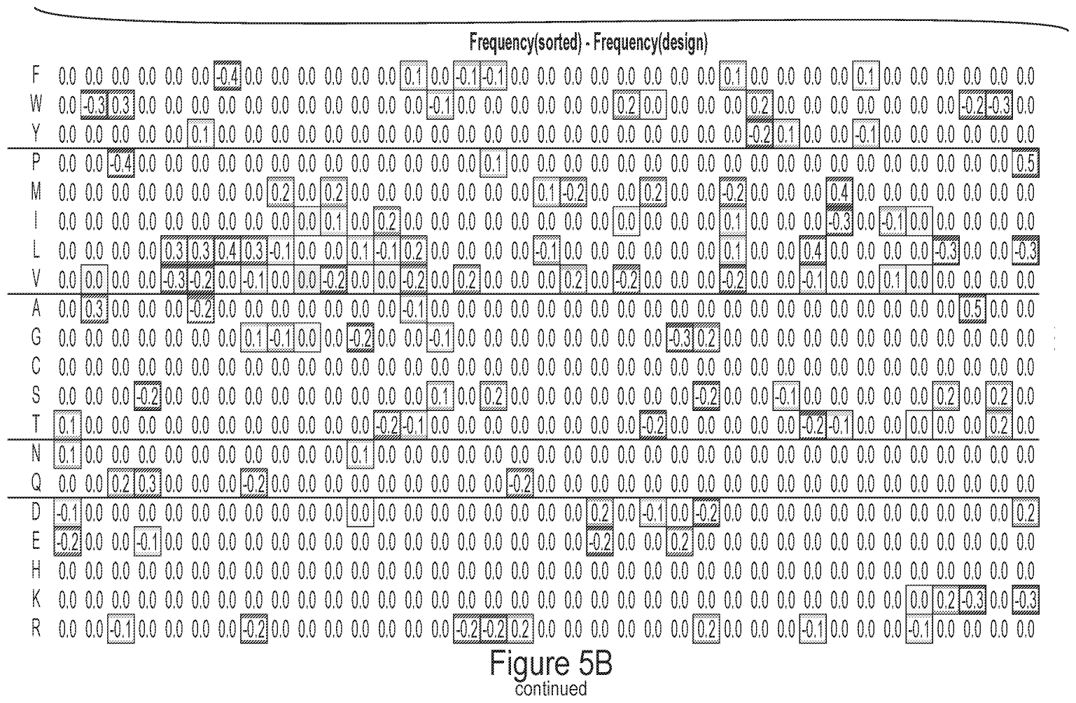

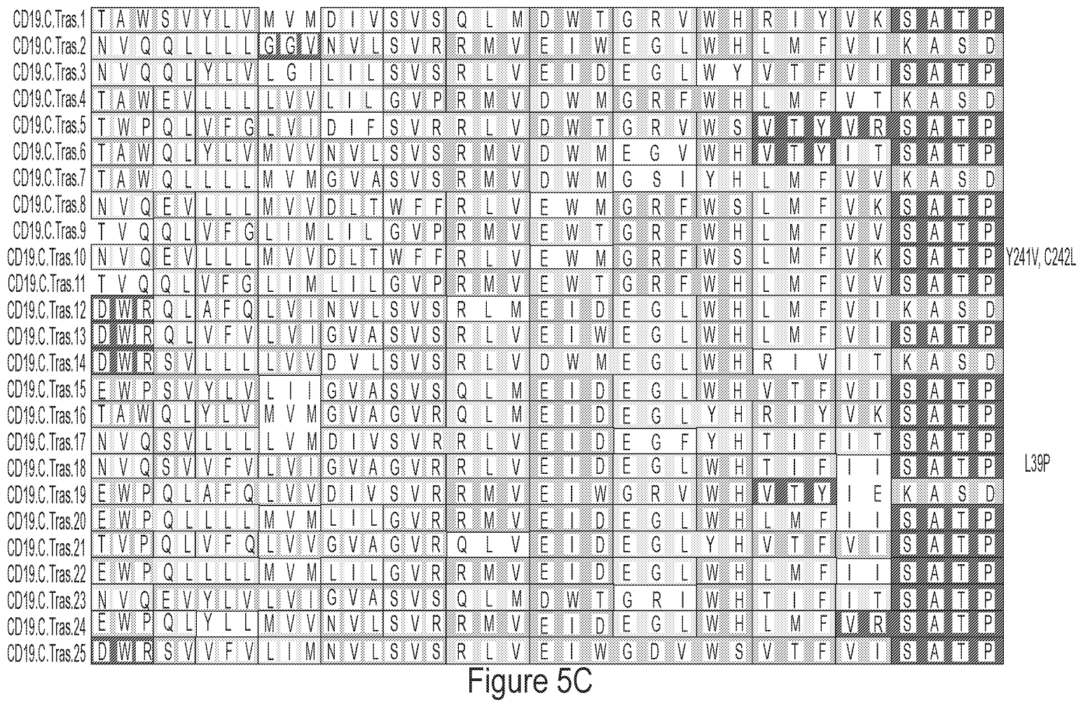

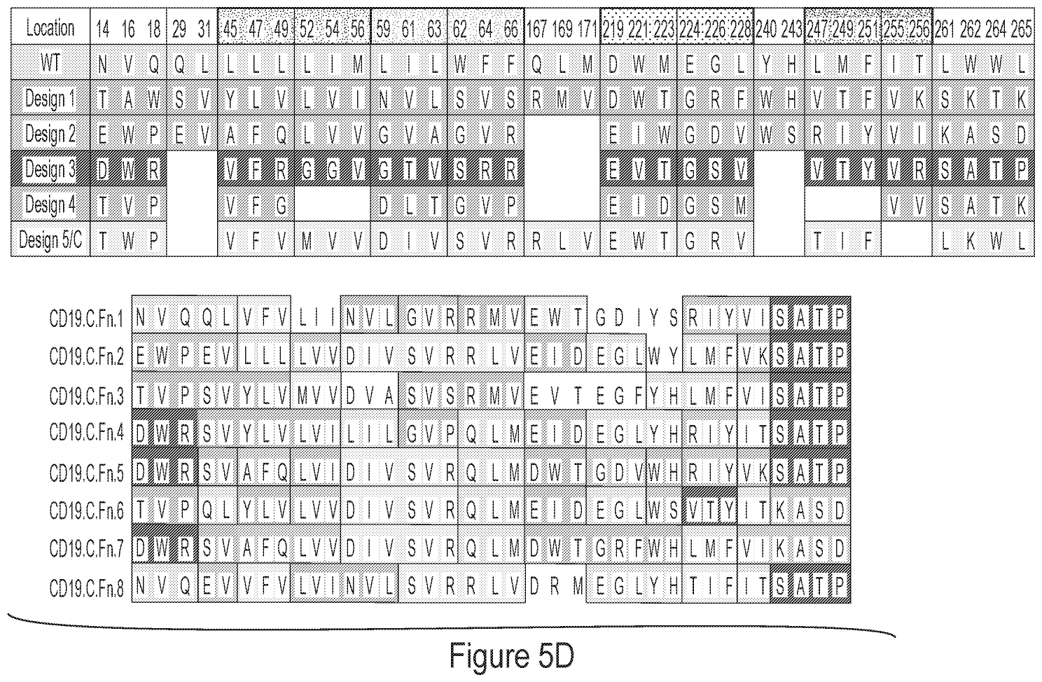

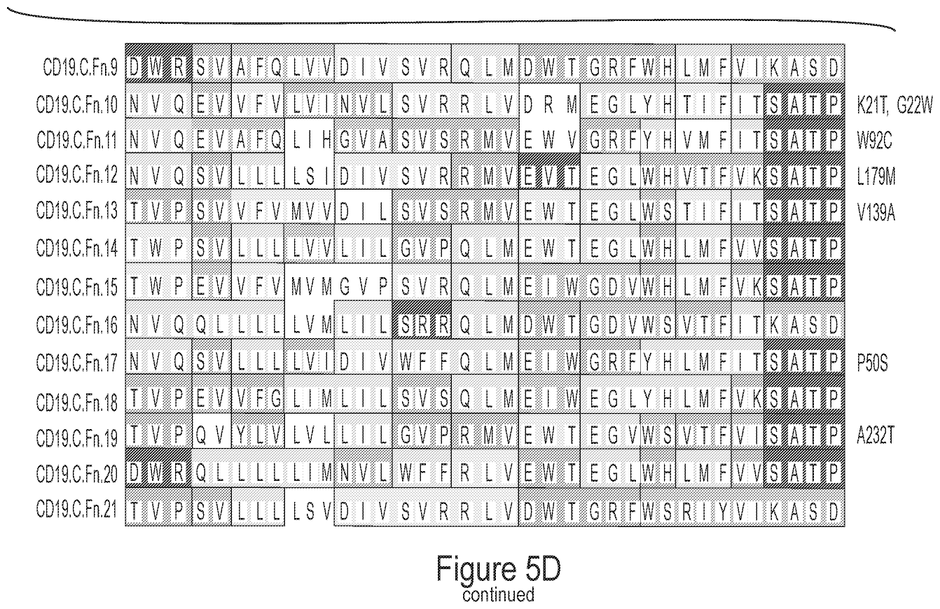

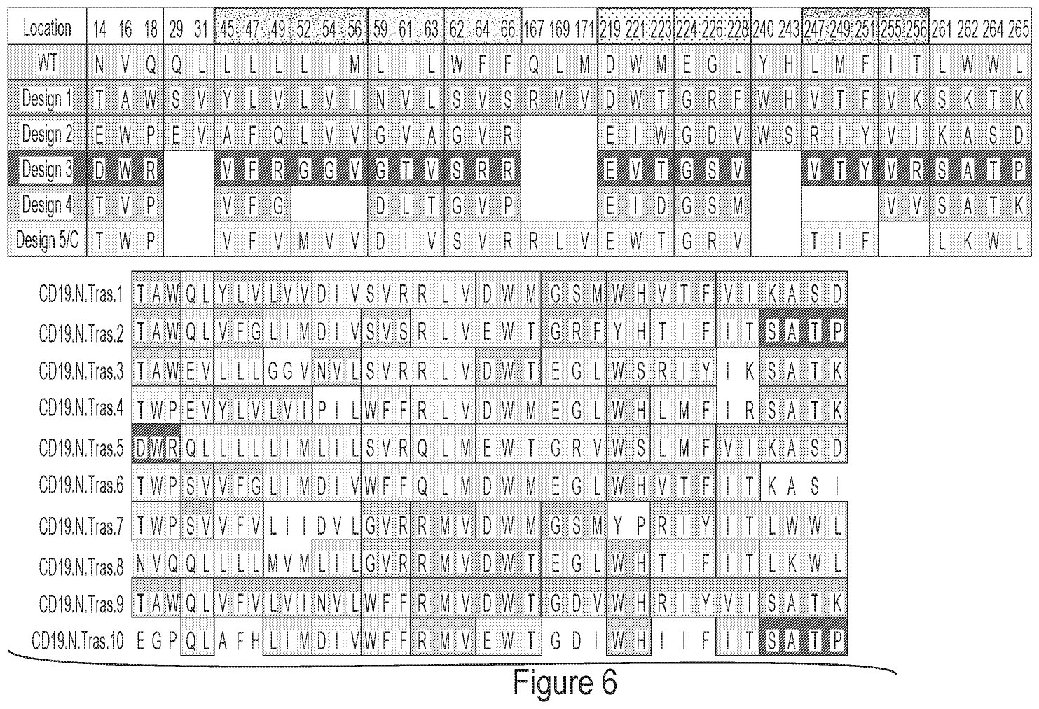

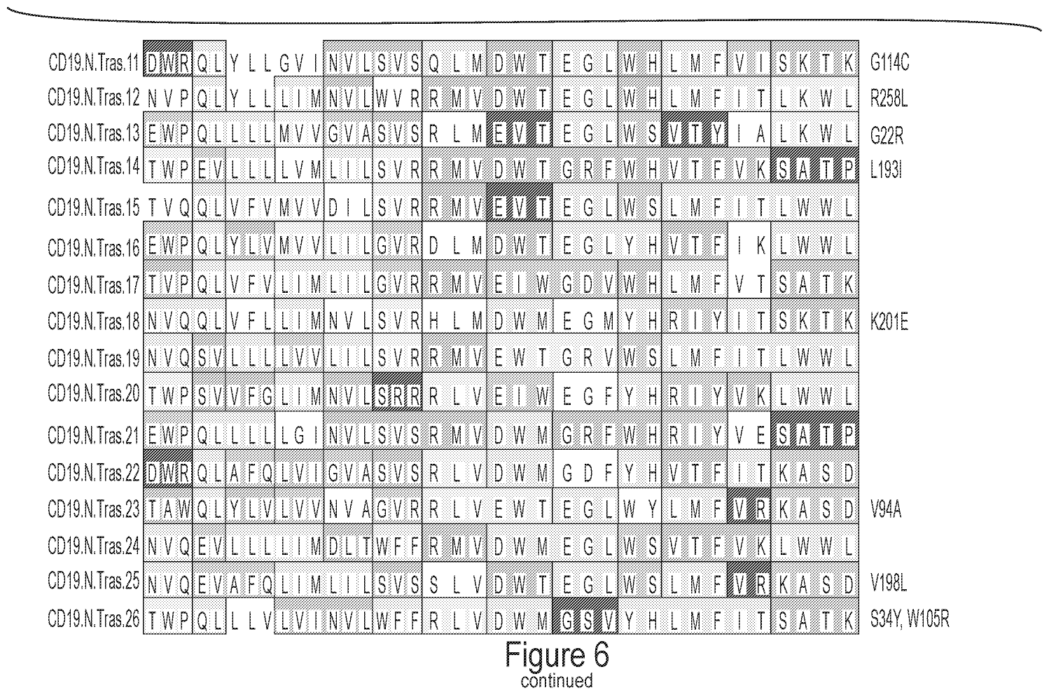

Thus, in some embodiments, a CD19 variant includes one or more of the amino acid substitutions of SEQ ID NO:2 listed in Table 1A, Table 1B, Table 2A, Table 2B, Table 3, Table 6, FIG. 3, FIG. 4B, FIG. 5A-FIG. 5D, or FIG. 6.

In some embodiments, a CD19 variant includes an amino acid substitution at one or more of the following amino acid positions of SEQ ID NO:2: 2, 3, 7, 8, 9, 10, 11, 12, 13, 14, 15, 16, 18, 20, 22, 25, 28, 29, 30, 31, 32, 33, 34, 38, 39, 45, 47, 49, 52, 53, 54, 55, 56, 57, 58, 59, 61, 62, 63, 64, 66, 68, 70, 72, 84, 90, 93, 94, 99, 100, 105, 108, 111, 113, 114, 115, 122, 123, 124, 125, 127, 130, 131, 132, 135, 138, 139, 140, 141, 142, 143, 144, 145, 146, 148, 149, 154, 167, 169, 171, 185, 189, 193, 194, 196, 198, 202, 204, 206, 207, 209, 211, 212, 213, 215, 216, 217, 219, 220, 221, 222, 223, 224, 225, 226, 228, 229, 230, 232, 235, 240, 243, 247, 249, 250, 251, 252, 253, 255, 256, 257, 258, 259, 260, 261, 262, 264, 265, 269, or 271. Exemplary amino acid substitutions at these positions are shown in Table 1A, Table 2A, Table 3, Table 6, FIG. 3, FIG. 4B, FIG. 5A-FIG. 5D, or FIG. 6. In some embodiments, a CD19 variant exhibits increased binding (relative to WT CD19) to an anti-CD19 antibody (e.g., FMC63 or 4G7); exhibits improved resistance (relative to WT CD19) to proteolysis (e.g., using a standard assay); and/or exhibits improved expression (relative to WT CD19) under thermal stress (e.g., using a standard assay).

In some embodiments, a CD19 variant includes one or more of the following amino acid substitutions at one or more of the following positions, as shown in Table 6:

TABLE 6

| Position of | Amino Acid Substitution |
|---|---|
| SEQ ID NO: 2 | (“*” denotes deletion) |
| 1 | F or I |
| 5 | V |
| 10 | M, R, Q, T, N, F, K, H, L, A, I, W, V, or Y |
| 13 | Q |
| 14 | T or D |
| 16 | F, W, Y, L, or M |
| 18 | P or L |
| 20 | G, P, D, S, N, or A |
| 21 | G or P |
| 23 | P, V, or R |
| 25 | L |
| 28 | D or Y |
| 29 | E |
| 30 | E |
| 31 | I, V, N, or M |
| 32 | E |
| 36 | D or G |
| 37 | D |
| 39 | N, D, E, H, S, T, or Q |
| 41 | G |
| 45 | Y or V |
| 47 | F, W, M, G, or T |
| 49 | A, C, or V |
| 52 | V |
| 53 | A |
| 54 | C or M |
| 55 | F, L, V, M, Y, A, P, or W |
| 56 | V, I, L, Q, or F |
| 57 | T, G, E, D, S, A, N, or Q |
| 59 | N, G, E, S, D, K, H, T, Q, A, R, M, V, W, or P |
| 60 | G |
| 61 | T |
| 62 | D, E, T, S, R, M, or Q |
| 63 | T, V, or A |
| 64 | T, V, G, A, R, E, S, Q, K, I, H, M, or Y |
| 66 | H, K, R, T, A, Q, S, P, G, or N |
| 70 | C, H, or A |
| 71 | D, S, or E |
| 79 | Y |
| 81 | P |
| 84 | M |
| 85 | A |
| 86 | D |
| 87 | N, G, H, F, or K |
| 89 | Y |
| 91 | A |
| 95 | S |
| 110 | K |
| 112 | D, N, H, or E |
| 113 | K or Y |
| 116 | N or Q |
| 126 | H |
| 129 | H |
| 130 | P or H |
| 132 | N or R |
| 133 | D or K |
| 139 | Y, T, or F |
| 140 | H |
| 141 | K, M, or N |
| 146 | Q or V |
| 147 | L, R, M, Q, or K |
| 152 | I, L, or V |
| 157 | D |
| 158 | N |
| 159 | H or P |
| 160 | L |
| 161 | D, N, or S |
| 162 | H |
| 167 | R or K |
| 171 | V or I |
| 177 | A |
| 178 | K, A, R, G, S, M, E, V, or N |
| 179 | I |
| 183 | I |
| 187 | A |
| 192 | W |
| 196 | L, M, V, I, or W |
| 197 | W |
| 199 | H |
| 201 | H or R |
| 205 | Q, V, L, D, A, or T |
| 208 | F or T |
| 210 | W |
| 212 | S, F, A, T, L, M, G, C, V, W, or P |
| 214 | * |
| 215 | Y, W, M, or A |
| 216 | H |
| 217 | Q, M, or L |
| 219 | E or I |
| 221 | V, I, or Y |
| 223 | T, D, W, N, S, Y, M, or F |
| 224 | D or G |
| 225 | D, N, S, or E |
| 226 | T, S, A, Q, M, Y, K, N, or E |
| 227 | I |
| 228 | V, M, or T |
| 231 | N or H |
| 232 | V |
| 234 | K, Q, R, M, or P |
| 235 | A or F |
| 239 | M or L |
| 240 | W |
| 243 | T, K, S, Q, M, or R |
| 247 | T or R |
| 250 | E or A |
| 252 | K, Q, I, V, E, or R |
| 253 | F |
| 254 | W |
| 255 | V |
| 256 | K |
| 257 | K, R, P, Q, or H |
| 258 | K |
| 259 | * |
| 260 | K, R, N, S, G, T, P, Y, or * |
| 261 | K, N, T, Q, W, R, S, A, G, P, H, or * |
| 262 | D, H, R, P, E, S, Q, N, T, A, K, N, I, M, Y, or G |
| 263 | K, P, R, or N |
| 264 | K, N, T, D, H, Q, G, S, P, R, E, A, or Y |
| 265 | P, K, G, R, or D |
| 266 | N, K, S, R, E, H, Q, G, D, A, or P |
| 268 | W, M, L, K, R, Q, or G |
| 269 | I, V, L, M, F, R, K, W, or T |
| 270 | N, P, or Q |
| 271 | K, H, or Q |

In some embodiments, a CD19 variant includes amino acid substitutions at one or more of the following sets of amino acid positions of SEQ ID NO:2: 5/7/9; 14/16/18; 29/31; 29/31/33; 35/37/39; 45/47/49; 52/54/56; 59/61/63; 62/64/66; 76/78/80; 86/88/90; 167/169/171; 175/177/179; 193/195/197; 206/208/210; 207/209/211; 219/221/223; 240/243; 224/226/228; 247/249/251; 253/255/256; 255/256; or 261/262/264/265. Exemplary amino acid substitutions at these sets of positions are shown in Table 1B, Table 2B, Table 3, Table 6, FIG. 4B, FIG. 3, FIG. 5A, FIG. 5B, FIG. 5C, FIG. 5D, or FIG. 6.

TABLE 1A
Single mutants with improved binding to FMC63 and 4G7:
Single Mutants

| FMC63 Binding | | | | 4G7 Binding | | | |
|---|---|---|---|---|---|---|---|
| Location | WT | Mutation | Score | Location | WT | Mutation | Score |
| 264 | W | N | 9. ± 1.3 | 59 | L | N | 13.5 ± 2.9 |
| 262 | W | D | 8.3 ± .8 | 59 | L | G | 10.6 ± .3 |
| 262 | W | K | 8.2 ± .7 | 59 | L | E | 10.6 ± .5 |
| 264 | W | S | 7.6 ± .5 | 59 | L | D | 10.9 ± .8 |
| 262 | W | R | 7.4 ± .3 | 59 | L | S | 10.1 ± .4 |
| 264 | W | P | 8.6 ± 1.5 | 62 | W | E | 10. ± .7 |
| 228 | L | V | 7.4 ± .4 | 57 | R | D | 10.1 ± .9 |
| 264 | W | K | 8. ± 1. | 57 | R | T | 9.6 ± .6 |
| 264 | W | G | 7.2 ± .3 | 59 | L | H | 9.6 ± .6 |
| 260 | V | K | 7.5 ± .7 | 16 | V | F | 9.8 ± 1. |
| 262 | W | P | 7.4 ± .7 | 62 | W | D | 10.8 ± 2. |
| 226 | G | T | 7.3 ± .6 | 64 | F | V | 8.9 ± .4 |
| 262 | W | S | 7. ± .3 | 57 | R | S | 8.8 ± .4 |
| 264 | W | Q | 7.4 ± .8 | 56 | M | I | 8.8 ± .5 |
| 262 | W | E | 6.9 ± .3 | 31 | L | I | 8.9 ± .6 |
| 264 | W | R | 6.9 ± .3 | 59 | L | K | 9.5 ± 1.3 |
| 260 | V | R | 6.7 ± .3 | 64 | F | G | 8.4 ± .2 |
| 262 | W | A | 6.9 ± .5 | 39 | L | D | 8.5 ± .5 |
| 264 | W | T | 7.2 ± .8 | 59 | L | Q | 8.5 ± .5 |
| 223 | M | T | 6.7 ± .4 | 59 | L | T | 8.8 ± .9 |
| 243 | H | T | 7.1 ± .9 | 57 | R | G | 8.1 ± .2 |
| 262 | W | Q | 6.8 ± .6 | 20 | L | G | 8.1 ± .2 |
| 212 | K | S | 6.8 ± .6 | 14 | N | T | 8.3 ± .5 |
| 262 | W | G | 6.4 ± .2 | 45 | L | Y | 9. ± 1.2 |
| 59 | L | G | 6.5 ± .3 | 64 | F | T | 8.8 ± 1.3 |
| 232 | A | V | 6.3 ± .2 | 57 | R | E | 8.2 ± .7 |
| 240 | Y | W | 6.3 ± .3 | 66 | F | R | 7.8 ± .3 |
| 16 | V | F | 7. ± 1. | 66 | F | K | 8.2 ± .8 |
| 215 | R | W | 7.1 ± 1.1 | 18 | Q | P | 7.6 ± .5 |
| 255 | I | V | 6.4 ± .4 | 55 | H | F | 8.2 ± 1.3 |
| 269 | G | I | 6.7 ± .8 | 64 | F | A | 7.2 ± .4 |
| 59 | L | S | 6.4 ± .4 | 16 | V | Y | 8.2 ± 1.5 |
| 59 | L | E | 6.3 ± .5 | 55 | H | L | 7.1 ± .5 |
| 262 | W | H | 6.7 ± 1. | 59 | L | A | 7.1 ± .5 |
| 226 | G | S | 6.1 ± .4 | 56 | M | V | 6.8 ± .3 |
| 264 | W | A | 6.3 ± .5 | 57 | R | N | 8.3 ± 2.1 |
| 64 | F | G | 6. ± .2 | 20 | L | P | 6.5 ± .3 |
| 66 | F | R | 6. ± .3 | 31 | L | V | 6.2 ± .3 |
| 269 | G | L | 6.1 ± .4 | 55 | H | Y | 6.4 ± .5 |
| 62 | W | E | 6.4 ± .7 | 64 | F | E | 6.4 ± .6 |
| 16 | V | W | 6.7 ± 1. | 262 | W | D | 6.6 ± .8 |
| 257 | A | R | 6.1 ± .5 | 262 | W | K | 6.5 ± .7 |
| 264 | W | D | 6.2 ± .6 | 55 | H | V | 6.1 ± .3 |
| 261 | L | K | 6.2 ± .6 | 262 | W | R | 5.9 ± .3 |
| 64 | F | V | 6. ± .4 | 264 | W | K | 6.6 ± 1. |
| 257 | A | K | 6.1 ± .5 | 66 | F | A | 6. ± .4 |
| 260 | V | S | 5.8 ± .3 | 64 | F | Q | 6.1 ± .6 |
| 212 | K | A | 6.1 ± .6 | 262 | W | S | 5.8 ± .3 |
| 57 | R | G | 5.7 ± .2 | 262 | W | P | 6. ± .7 |
| 20 | L | G | 5.7 ± .2 | 66 | F | T | 6.2 ± .8 |

TABLE 1B
Triple mutants with improved binding to FMC63 and 4G7:
Triple Mutants

| FMC63 Binding | | | | 4G7 Binding | | | |
|---|---|---|---|---|---|---|---|
| Location | WT | Mutations | Score | Location | WT | Mutations | Score |
| 62, 64, 66 | W, F, F | S, V, S | 15.4 ± 2.8 | 224, 226, 228 | E, G, L | D, N, F | 12.2 ± 2.4 |
| 62, 64, 66 | W, F, F | G, V, R | 14.1 ± 1.9 | 224, 226, 228 | E, G, L | G, S, M | 13. ± 3.3 |
| 62, 64, 66 | W, F, F | S, R, R | 13.7 ± 1.9 | 224, 226, 228 | E, G, L | G, R, I | 11.6 ± 1.9 |
| 62, 64, 66 | W, F, F | T, V, P | 16.6 ± 5.5 | 224, 226, 228 | E, G, L | G, S, L | 12.9 ± 3.3 |
| 224, 226, 228 | E, G, L | G, R, F | 12.8 ± 1.7 | 224, 226, 228 | E, G, L | G, T, V | 11.9 ± 2.4 |
| 62, 64, 66 | W, F, F | S, R, S | 13.2 ± 2.1 | 224, 226, 228 | E, G, L | E, A, I | 12.8 ± 3.3 |
| 62, 64, 66 | W, F, F | E, R, P | 16.5 ± 5.5 | 224, 226, 228 | E, G, L | E, N, V | 11.3 ± 1.9 |
| 62, 64, 66 | W, F, F | E, W, R | 14.2 ± 3.2 | 224, 226, 228 | E, G, L | G, A, T | 11.6 ± 2.4 |
| 62, 64, 66 | W, F, F | E, V, V | 13.4 ± 2.5 | 224, 226, 228 | E, G, L | G, E, V | 10.2 ± .9 |
| 224, 226, 228 | E, G, L | G, E, I | 14.3 ± 3.4 | 224, 226, 228 | E, G, L | G, A, V | 10. ± .8 |
| 62, 64, 66 | W, F, F | D, L, P | 16.3 ± 5.5 | 224, 226, 228 | E, G, L | G, R, Y | 11.5 ± 2.4 |
| 224, 226, 228 | E, G, L | G, D, V | 12. ± 1.2 | 224, 226, 228 | E, G, L | G, T, I | 10.2 ± 1.1 |
| 62, 64, 66 | W, F, F | A, R, L | 13.1 ± 2.3 | 224, 226, 228 | E, G, L | E, R, V | 10.5 ± 1.4 |
| 62, 64, 66 | W, F, F | W, S, S | 14. ± 3.2 | 224, 226, 228 | E, G, L | G, D, V | 10.3 ± 1.2 |
| 62, 64, 66 | W, F, F | T, R, Q | 16.3 ± 5.5 | 224, 226, 228 | E, G, L | G, R, F | 10.6 ± 1.7 |
| 62, 64, 66 | W, F, F | R, V, R | 12.3 ± 1.6 | 224, 226, 228 | E, G, L | E, S, V | 9.9 ± 1. |
| 59, 61, 63 | L, I, L | N, V, L | 13.9 ± 3.2 | 224, 226, 228 | E, G, L | G, R, V | 9.8 ± .9 |
| 62, 64, 66 | W, F, F | E, E, V | 16.2 ± 5.5 | 224, 226, 228 | E, G, L | G, R, L | 9.6 ± .7 |
| 62, 64, 66 | W, F, F | Y, S, R | 13.7 ± 3.2 | 224, 226, 228 | E, G, L | D, A, W | 12.2 ± 3.3 |
| 62, 64, 66 | W, F, F | D, V, M | 16. ± 5.6 | 219, 221, 223 | D, W, M | E, I, W | 12.2 ± 3.3 |
| 62, 64, 66 | W, F, F | W, V, A | 13.7 ± 3.2 | 224, 226, 228 | E, G, L | G, T, E | 10.4 ± 1.7 |
| 62, 64, 66 | W, F, F | T, V, F | 13.6 ± 3.2 | 224, 226, 228 | E, G, L | D, A, V | 12.1 ± 3.3 |
| 224, 226, 228 | E, G, L | E, E, V | 11.6 ± 1.2 | 224, 226, 228 | E, G, L | G, S, V | 9.8 ± 1.1 |
| 62, 64, 66 | W, F, F | R, A, F | 15.9 ± 5.6 | 224, 226, 228 | E, G, L | G, N, L | 10.3 ± 1.7 |
| 224, 226, 228 | E, G, L | G, S, M | 13.8 ± 3.4 | 224, 226, 228 | E, G, L | G, G, I | 9.8 ± 1.1 |
| 224, 226, 228 | E, G, L | D, V, V | 13.8 ± 3.4 | 224, 226, 228 | E, G, L | G, V, V | 9.6 ± 1. |
| 62, 64, 66 | W, F, F | E, C, S | 12.6 ± 2.3 | 224, 226, 228 | E, G, L | G, D, L | 9.8 ± 1.3 |
| 62, 64, 66 | W, F, F | E, R, R | 12.4 ± 2.1 | 224, 226, 228 | E, G, L | G, R, M | 9.7 ± 1.2 |
| 62, 64, 66 | W, F, F | D, V, R | 12.6 ± 2.3 | 224, 226, 228 | E, G, L | G, N, T | 11.9 ± 3.3 |
| 224, 226, 228 | E, G, L | G, A, T | 12.7 ± 2.4 | 224, 226, 228 | E, G, L | G, E, H | 9.8 ± 1.4 |
| 62, 64, 66 | W, F, F | S, V, A | 13.5 ± 3.2 | 224, 226, 228 | E, G, L | D, R, L | 9.9 ± 1.5 |
| 224, 226, 228 | E, G, L | G, V, V | 11.2 ± 1. | 224, 226, 228 | E, G, L | G, D, C | 11.6 ± 3.3 |
| 62, 64, 66 | W, F, F | T, A, S | 15.7 ± 5.6 | 224, 226, 228 | E, G, L | E, T, V | 10. ± 1.7 |
| 62, 64, 66 | W, F, F | H, F, R | 12.4 ± 2.3 | 224, 226, 228 | E, G, L | G, E, I | 11.6 ± 3.3 |
| 224, 226, 228 | E, G, L | G, R, V | 11. ± .9 | 224, 226, 228 | E, G, L | G, A, H | 11.6 ± 3.3 |
| 62, 64, 66 | W, F, F | T, V, R | 12.2 ± 2.1 | 224, 226, 228 | E, G, L | G, E, R | 9.1 ± 1. |
| 62, 64, 66 | W, F, F | T, M, S | 15.7 ± 5.6 | 224, 226, 228 | E, G, L | D, L, I | 9.4 ± 1.3 |
| 224, 226, 228 | E, G, L | G, S, V | 11.2 ± 1.1 | 224, 226, 228 | E, G, L | G, Y, V | 11.4 ± 3.3 |
| 62, 64, 66 | W, F, F | S, V, R | 11.8 ± 1.7 | 224, 226, 228 | E, G, L | G, M, V | 11.4 ± 3.3 |
| 62, 64, 66 | W, F, F | H, V, S | 14. ± 4. | 224, 226, 228 | E, G, L | G, S, I | 11.3 ± 3.3 |
| 224, 226, 228 | E, G, L | G, G, I | 11.2 ± 1.2 | 224, 226, 228 | E, G, L | G, K, M | 11.3 ± 3.3 |
| 59, 61, 63 | L, I, L | N, I, I | 15.6 ± 5.6 | 224, 226, 228 | E, G, L | G, S, G | 8.7 ± .8 |
| 62, 64, 66 | W, F, F | S, F, N | 15.6 ± 5.6 | 224, 226, 228 | E, G, L | D, L, V | 11.2 ± 3.3 |
| 62, 64, 66 | W, F, F | N, L, P | 15.6 ± 5.6 | 224, 226, 228 | E, G, L | G, C, V | 9. ± 1.1 |
| 62, 64, 66 | W, F, F | D, V, S | 13.3 ± 3.2 | 224, 226, 228 | E, G, L | G, E, W | 9. ± 1.1 |
| 224, 226, 228 | E, G, L | G, A, V | 10.8 ± .8 | 224, 226, 228 | E, G, L | G, Q, M | 11.2 ± 3.3 |
| 62, 64, 66 | W, F, F | W, T, A | 13.9 ± 4. | 224, 226, 228 | E, G, L | D, Y, T | 9.7 ± 1.9 |
| 62, 64, 66 | W, F, F | D, A, S | 15.5 ± 5.6 | 219, 221, 223 | D, W, M | E, V, N | 11.1 ± 3.3 |
| 224, 226, 228 | E, G, L | G, R, I | 12. ± 2. | 224, 226, 228 | E, G, L | G, A, M | 8.9 ± 1.2 |
| 62, 64, 66 | W, F, F | G, V, K | 12.3 ± 2.3 | 219, 221, 223 | D, W, M | E, I, D | 9.6 ± 1.9 |

TABLE 2A
Single mutants with improved resistance to proteolysis
Single Mutants

| Protease Stability-High | | | | Protease Stability-Low | | | |
|---|---|---|---|---|---|---|---|
| Location | WT | Mutation | Score | Location | WT | Mutation | Score |
| 132 | L | Q | 9.9 ± 2.1 | 149 | E | Y | 9.5 ± 3. |
| 261 | L | F | 9.7 ± 2.9 | 141 | A | I | 9.3 ± 3. |
| 149 | E | Y | 9. ± 3. | 209 | L | T | 7.5 ± 2.1 |
| 127 | S | Q | 8.1 ± 2.1 | 113 | L | C | 7.5 ± 2.2 |
| 130 | G | Q | 8.1 ± 2.1 | 9 | V | N | 7.8 ± 3. |
| 211 | L | T | 7.9 ± 2.1 | 122 | S | N | 6.1 ± 1.5 |
| 225 | T | Y | 8.7 ± 2.9 | 217 | A | W | 6.4 ± 1.7 |
| 209 | L | T | 7.9 ± 2.1 | 252 | H | M | 7.5 ± 3. |
| 140 | W | H | 7.6 ± 2.2 | 196 | T | M | 7.4 ± 3. |
| 125 | P | N | 6.5 ± 1.5 | 58 | P | Q | 7.4 ± 3. |
| 100 | G | D | 5.7 ± .9 | 72 | M | E | 7.4 ± 3. |
| 223 | M | F | 7.6 ± 3. | 258 | R | Y | 6.4 ± 2.1 |
| 250 | S | N | 7.6 ± 3. | 70 | Q | T | 7.2 ± 3. |
| 198 | V | Y | 6.3 ± 1.8 | 220 | M | C | 5.7 ± 1.5 |
| 108 | S | H | 5.7 ± 1.3 | 256 | T | D | 6.3 ± 2.1 |
| 217 | A | W | 6.1 ± 1.8 | 235 | Q | Y | 7.1 ± 3. |
| 193 | L | Q | 5.4 ± 1.2 | 257 | A | I | 7.1 ± 3. |
| 54 | I | K | 5.9 ± 1.7 | 261 | L | T | 7.1 ± 3. |
| 123 | E | Y | 4.9 ± 1. | 13 | D | M | 7.1 ± 3. |
| 256 | T | D | 6.1 ± 2.2 | 32 | T | H | 7.1 ± 3. |
| 262 | W | K | 6.1 ± 2.2 | 53 | G | K | 7.1 ± 3. |
| 141 | A | Y | 5.2 ± 1.2 | 54 | I | K | 5.8 ± 1.7 |
| 143 | D | M | 4.6 ± .6 | 11 | E | F | 5.5 ± 1.5 |
| 130 | G | D | 4.9 ± 1. | 194 | S | I | 5.7 ± 1.8 |
| 143 | D | N | 4.9 ± 1. | 2 | E | H | 6.1 ± 2.1 |
| 194 | S | W | 5.4 ± 1.5 | 28 | T | H | 6.9 ± 3. |
| 100 | G | M | 5.7 ± 1.8 | 271 | W | H | 5.6 ± 1.8 |
| 202 | G | M | 6. ± 2.2 | 204 | K | P | 6.8 ± 3. |
| 70 | Q | T | 6.7 ± 3. | 84 | S | H | 5.5 ± 1.7 |
| 212 | K | C | 5.5 ± 1.8 | 34 | S | E | 5.1 ± 1.4 |
| 111 | G | K | 5.1 ± 1.4 | 3 | E | I | 5.8 ± 2.1 |
| 131 | K | I | 5.4 ± 1.8 | 47 | L | W | 5.8 ± 2.1 |
| 130 | G | N | 4.8 ± 1.2 | 222 | V | D | 5.3 ± 1.8 |
| 146 | E | C | 4.2 ± .6 | 11 | E | L | 4.1 ± .6 |
| 220 | M | C | 5.1 ± 1.6 | 22 | G | I | 5.6 ± 2.1 |
| 94 | V | Y | 4.9 ± 1.4 | 25 | D | W | 5.2 ± 1.8 |
| 49 | L | S | 5.6 ± 2.1 | 72 | M | A | 5.2 ± 1.8 |
| 56 | M | C | 4.5 ± 1. | 148 | W | H | 4.7 ± 1.3 |
| 262 | W | Y | 5.6 ± 2.2 | 15 | A | C | 4.3 ± .9 |
| 115 | C | N | 4.3 ± .9 | 84 | S | K | 4.1 ± .9 |
| 93 | T | D | 4. ± .6 | 14 | N | C | 3.9 ± .7 |
| 124 | G | F | 4.5 ± 1.2 | 8 | K | F | 4.9 ± 1.8 |
| 132 | L | C | 4.3 ± 1. | 30 | Q | H | 4.1 ± .9 |
| 145 | P | I | 4. ± .8 | 49 | L | S | 5.3 ± 2.2 |
| 258 | R | E | 5.4 ± 2.2 | 38 | P | N | 4.7 ± 1.5 |
| 29 | Q | I | 4.6 ± 1.4 | 15 | A | H | 4.8 ± 1.8 |
| 52 | L | N | 6.2 ± 3. | 243 | H | S | 3.9 ± .9 |
| 113 | L | K | 4.6 ± 1.5 | 230 | P | N | 4.4 ± 1.4 |
| 185 | P | E | 4.7 ± 1.6 | 68 | V | F | 4.1 ± 1.1 |
| 141 | A | L | 3.9 ± .8 | 61 | I | K | 5.1 ± 2.2 |

TABLE 2B
Triple mutants with improved resistance to proteolysis
Triple Mutants
Protease Stability

| Location | WT | Mutations | Score |
|---|---|---|---|
| 62, 64, 66 | W, F, F | Y, S, T | 13.8 ± 5.6 |
| 62, 64, 66 | W, F, F | V, G, D | 11.9 ± 4. |
| 35, 37, 39 | R, S, L | E, G, W | 13.1 ± 5.6 |
| 45, 47, 49 | L, L, L | V, C, R | 13. ± 5.7 |
| 59, 61, 63 | L, I, L | Y, V, S | 11.4 ± 4.1 |
| 52, 54, 56 | L, I, M | E, E, G | 11.3 ± 4.1 |
| 52, 54, 56 | L, I, M | G, A, M | 12.9 ± 5.7 |
| 35, 37, 39 | R, S, L | S, C, S | 12.8 ± 5.7 |
| 45, 47, 49 | L, L, L | l, D, P | 12.8 ± 5.7 |
| 52, 54, 56 | L, I, M | E, V, V | 12.8 ± 5.7 |
| 52, 54, 56 | L, I, M | L, A, R | 12.8 ± 5.7 |
| 86, 88, 90 | K, W, P | G, M, Q | 12.8 ± 5.7 |
| 14, 16, 18 | N, V, Q | H, D, R | 12.5 ± 5.7 |
| 5, 7, 9 | L, V, V | S, R, E | 12.5 ± 5.7 |
| 45, 47, 49 | L, L, L | H, G, V | 12.5 ± 5.7 |
| 52, 54, 56 | L, I, M | Y, G, W | 12.5 ± 5.7 |
| 62, 64, 66 | W, F, F | S, K, S | 12.5 ± 5.7 |
| 76, 78, 80 | Y, C, P | F, S, G | 12.5 ± 5.7 |
| 86, 88, 90 | K, W, P | S, H, K | 12.5 ± 5.7 |
| 5, 7, 9 | L, V, V | E, W, A | 12.4 ± 5.7 |
| 52, 54, 56 | L, I, M | M, G, R | 12.4 ± 5.7 |
| 59, 61, 63 | L, I, L | S, Q, H | 12.4 ± 5.7 |
| 62, 64, 66 | W, F, F | P, W, G | 12.4 ± 5.7 |
| 193, 195, 197 | L, W, H | L, D, S | 12.4 ± 5.7 |
| 219, 221, 223 | D, W, M | V, G, W | 12.4 ± 5.7 |
| 59, 61, 63 | L, I, L | M, L, G | 10. ± 3.4 |
| 35, 37, 39 | R, S, L | S, C, R | 12.2 ± 5.7 |
| 45, 47, 49 | L, L, L | G, T, L | 12.2 ± 5.7 |
| 52, 54, 56 | L, I, M | R, V, P | 12.2 ± 5.7 |
| 52, 54, 56 | L, I, M | E, V, R | 12.2 ± 5.7 |
| 52, 54, 56 | L, I, M | Q, E, V | 12.2 ± 5.7 |
| 59, 61, 63 | L, I, L | K, M, A | 12.2 ± 5.7 |
| 59, 61, 63 | L, I, L | S, Q, R | 12.2 ± 5.7 |
| 62, 64, 66 | W, F, F | A, Q, T | 12.2 ± 5.7 |
| 86, 88, 90 | K, W, P | S, S, S | 12.2 ± 5.7 |
| 206, 208, 210 | L, S, E | K, L, R | 10.6 ± 4.1 |
| 206, 208, 210 | L, S, E | G, E, N | 12.2 ± 5.7 |
| 219, 221, 223 | D, W, M | E, G, L | 12.2 ± 5.7 |
| 5, 7, 9 | L, V, V | A, P, A | 12.1 ± 5.7 |
| 52, 54, 56 | L, I, M | S, R, F | 12.1 ± 5.7 |
| 52, 54, 56 | L, I, M | M, G, S | 12.1 ± 5.7 |
| 59, 61, 63 | L, I, L | T, L, G | 12.1 ± 5.7 |
| 59, 61, 63 | L, I, L | A, H, Q | 12.1 ± 5.7 |
| 59, 61, 63 | L, I, L | M, T, A | 12.1 ± 5.7 |
| 62, 64, 66 | W, F, F | V, E, E | 12.1 ± 5.7 |
| 86, 88, 90 | K, W, P | M, A, G | 12.1 ± 5.7 |
| 59, 61, 63 | L, I, L | S, Q, T | 9.7 ± 3.4 |
| 193, 195, 197 | L, W, H | V, G, F | 12. ± 5.7 |
| 206, 208, 210 | L, S, E | K, P, M | 12. ± 5.7 |
| 224, 226, 228 | E, G, L | G, F, A | 12. + 5.7 |

TABLE 3

| | Mutants with improved expression under thermal stress | | | | | | |
|---|---|---|---|---|---|---|---|
| | Triple Mutants | | | | Single Mutants | | |
| | Thermal Induction | | | | Thermal Induction | | |
| Location | WT | Mutations | Score | Location | WT | Mutation | Score |
| 247, 249, 251 | L, M, F | K, H, V | 12. ± 6.3 | 189 | S | H | 8.3 ± 3. |
| 253, 255, 256 | L, I, T | E, D, C | 9.5 ± 3.8 | 206 | L | C | 7.6 ± 3. |
| 167, 169, 171 | Q, L, M | M, R, M | 11.8 ± 6.3 | 216 | P | H | 7.6 ± 3. |
| 207, 209, 211 | L, L, L | G, K, W | 11.8 ± 6.3 | 256 | T | M | 6.7 ± 2.2 |
| 247, 249, 251 | L, M, F | K, G, V | 11.8 ± 6.3 | 94 | V | S | 5.4 ± 1.3 |
| 167, 169, 171 | Q, L, M | K, L, Q | 9.9 ± 4.6 | 261 | L | M | 5.6 ± 1.6 |
| 247, 249, 251 | L, M, F | K, A, D | 11.6 ± 6.3 | 213 | D | H | 5.4 ± 1.4 |
| 247, 249, 251 | L, M, F | I, G, T | 11.6 ± 6.3 | 99 | S | Q | 6.8 ± 2.9 |
| 167, 169, 171 | Q, L, M | S, G, M | 9.7 ± 4.6 | 229 | L | K | 5.6 ± 1.8 |
| 167, 169, 171 | Q, L, M | L, W, H | 11.4 ± 6.3 | 262 | W | H | 5.9 ± 2.2 |
| 247, 249, 251 | L, M, F | T, N, I | 11.4 ± 6.3 | 114 | G | E | 6.6 ± 2.9 |
| 247, 249, 251 | L, M, F | N, V, S | 11.4 ± 6.3 | 15 | A | E | 5.8 ± 2.1 |
| 247, 249, 251 | L, M, F | V, G, A | 8.3 ± 3.3 | 209 | L | H | 4.5 ± .9 |
| 253, 255, 256 | L, I, T | I, V, S | 9.5 ± 4.6 | 204 | K | D | 5.7 ± 2.2 |
| 175, 177, 179 | S, L, L | L, E, M | 11.2 ± 6.3 | 148 | W | P | 6.3 ± 2.9 |
| 193, 195, 197 | L, W, H | S, L, S | 11.2 ± 6.3 | 99 | S | P | 6.3 ± 2.9 |
| 207, 209, 211 | L, L, L | Y, P, G | 11.2 ± 6.3 | 207 | L | G | 3.8 ± .5 |
| 224, 226, 228 | E, G, L | A, L, L | 11.2 ± 6.3 | 90 | P | E | 5.4 ± 2.1 |
| 224, 226, 228 | E, G, L | E, L, A | 11.2 ± 6.3 | 206 | L | Q | 4.9 ± 1.6 |
| 253, 255, 256 | L, I, T | Q, C, G | 11.2 ± 6.3 | 138 | Y | E | 4.7 ± 1.5 |
| 253, 255, 256 | L, I, T | C, Q, S | 11.2 ± 6.3 | 99 | S | N | 6.1 ± 2.9 |
| 52, 54, 56 | L, I, M | G, G, M | 8.8 ± 4. | 10 | E | I | 5.2 ± 2.1 |
| 52, 54, 56 | L, I, M | E, W, V | 8.8 ± 4. | 142 | K | L | 4.7 ± 1.7 |
| 175, 177, 179 | S, L, L | R, T, M | 9.3 ± 4.6 | 52 | L | H | 6. ± 2.9 |
| 253, 255, 256 | L, I, T | D, S, W | 9.3 ± 4.6 | 135 | P | W | 5.1 ± 2.1 |
| 167, 169, 171 | Q, L, M | E, S, Y | 11. ± 6.3 | 144 | R | E | 4.3 ± 1.3 |
| 175, 177, 179 | S, L, L | R, T, P | 11. ± 6.3 | 139 | V | K | 5.9 ± 2.9 |
| 193, 195, 197 | L, W, H | P, A, R | 11. ± 6.3 | 209 | L | M | 4. ± 1.1 |
| 207, 209, 211 | L, L, L | L, M, R | 11. ± 6.3 | 259 | P | C | 4. ± 1.1 |
| 224, 226, 228 | E, G, L | T, W, G | 11. ± 6.3 | 52 | L | N | 5.9 ± 3. |
| 247, 249, 251 | L, M, F | S, T, V | 11. ± 6.3 | 131 | K | A | 5.8 ± 2.9 |
| 253, 255, 256 | L, I, T | D, Q, R | 11. ± 6.3 | 198 | V | N | 4.7 ± 1.9 |
| 253, 255, 256 | L, I, T | C, N, E | 11. ± 6.3 | 235 | Q | D | 4.4 ± 1.6 |
| 253, 255, 256 | L, I, T | K, D, D | 11. ± 6.3 | 207 | L | Q | 4. ± 1.3 |
| 253, 255, 256 | L, I, T | Q, G, C | 11. ± 6.3 | 194 | S | W | 4.2 ± 1.5 |
| 45, 47, 49 | L, L, L | G, E, H | 8.6 ± 4.1 | 52 | L | E | 3.9 ± 1.1 |
| 52, 54, 56 | L, I, M | L, E, V | 8.6 ± 4.1 | 209 | L | K | 3.8 ± 1.1 |
| 52, 54, 56 | L, I, M | C, G, V | 8.6 ± 4.1 | 140 | W | P | 5.6 ± 2.9 |
| 193, 195, 197 | L, W, H | A, G, L | 8.3 ± 3.9 | 142 | K | C | 5.5 ± 2.9 |
| 86, 88, 90 | K, W, P | T, T, G | 8.4 ± 4.1 | 7 | V | F | 5.5 ± 3. |
| 167, 169, 171 | Q, L, M | T, M, G | 10.7 ± 6.4 | 12 | G | C | 5.5 ± 3. |
| 175, 177, 179 | S, L, L | M, R, D | 10.7 ± 6.4 | 18 | Q | D | 5.5 ± 3. |
| 193, 195, 197 | L, W, H | Q, C, A | 10.7 ± 6.4 | 204 | K | E | 3.1 ± .6 |
| 193, 195, 197 | L, W, H | D, T, D | 10.7 ± 6.4 | 262 | W | A | 3.4 ± .9 |
| 207, 209, 211 | L, L, L | A, R, M | 10.7 ± 6.4 | 185 | P | W | 3.5 ± 1. |
| 207, 209, 211 | L, L, L | M, T, G | 10.7 ± 6.4 | 196 | T | H | 3.6 ± 1.1 |
| 219, 221, 223 | D, W, M | S, G, S | 10.7 ± 6.4 | 249 | M | K | 3.3 ± .8 |
| 247, 249, 251 | L, M, F | L, A, M | 10.7 ± 6.4 | 105 | W | S | 3.5 ± 1. |
| 247, 249, 251 | L, M, F | F, W, P | 10.7 ± 6.4 | 154 | C | T | 5.4 ± 2.9 |
| 253, 255, 256 | L, I, T | T, E, N | 10.7 ± 6.4 | 138 | Y | P | 4.5 ± 2.1 |

Anti-CLL-1 Antibodies

In some embodiments, an expressed biparatopic fusion protein can include two or more antibodies, or fragments thereof, that bind CLL-1. Human C-type lectin-like molecule-1 (CLL-1), also known as MICL or CLEC12A, is a type II transmembrane glycoprotein and member of the large family of C-type lectin-like receptors involved in immune regulation. CLL-1 has previously been identified from myeloid-derived cells. The intracellular domain of CLL-1 contains an immunotyrosine-based inhibition motif (ITIM) and a YXXM motif Phosphorylation of ITIM-containing receptors on a variety of cells results in inhibition of activation pathways through recruitment of protein tyrosine phosphatases SHP-1, SHP-2 and SHIP. The YXXM motif has a potential SH2 domain-binding site for the p85 subunit of PI-3 kinase, 13 which has been implicated in cellular activation pathways, revealing a potential dual role of CLL-1 as an inhibitory and activating molecule on myeloid cells. Indeed, association of CLL-1 with SHP-1 and SHP-2 has been demonstrated experimentally in transfected and myeloid-derived cell lines.

Antibodies include, e.g., intact IgG, IgE and IgM, bi- or multi-specific antibodies (e.g., biparatopic, Zybodies®, etc.), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-Bodies®, Affibodies®, a TrimerX®, MicroProteins, Fynomers®, Centyrins®, and a KALBITOR®.

In some embodiments, a biparatopic fusion protein includes at least two antibodies (or antibody fragments), each of which binds CLL-1/CLEC12A. Anti-CLL-1 antibodies (and fragments, e.g., scFv) are known in the art. In some embodiments, a biparatopic fusion protein includes one or more scFvs that bind CLL-1/CLEC12A, e.g., sequences and antibodies or fragments thereof disclosed in U.S. Pat. No. 7,741,443; Kenderian et al., Blood 2016 128:766; Laborda et al., Int. J. Mol. Sci. 2017, 18, 2259; Tashiro et al., Mol. Ther. Vol. 25 No 9, 2202-2213, 2017; Wang et al. J Hematol Oncol. 2018 Jan. 10; 11(1):7; Lu et al., Angew Chem Int Ed Engl. 2014 Sep. 8; 53(37):9841-5; International Patent Application WO2016120219; International Patent Application WO2013169625.

Anti-CLL-1 Single Domain Antibodies

In some embodiments, an antibody is a single domain antibody. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art known, or any future single domain antibodies. Single domain antibodies may be derived from any species including, but not limited to mouse, human, camel, llama, goat, rabbit, bovine. According to one aspect of the disclosure, a single domain antibody as used herein is a naturally occurring single domain antibody known as heavy chain antibody devoid of light chains. Such single domain antibodies are disclosed in, e.g., WO 94/04678. Such variable domains derived from a heavy chain antibody naturally devoid of light chain is referred to herein as a “VHH” or “nanobody”. Such a VHH molecule can be derived from antibodies raised in Camelidae species, for example in camel, dromedary, llama, vicuna, alpaca and guanaco. Other species besides Camelidae (e.g., Homo sapiens) may produce heavy chain antibodies naturally devoid of light chain; such VHHs are within the scope of the disclosure.

The amino acid residues of VHH domains from Camelids are numbered according to the general numbering for VH domains given by Kabat et al., “Sequence of proteins of immunological interest”, US Public Health Services, NIH (Bethesda, Md.), Publication No 91-3242 (1991); see also Riechmann et al., J. Immunol. Methods 231:25-38 (1999). According to this numbering, FR1 comprises the amino acid residues at positions 1-30, CDR1 comprises the amino acid residues at positions 31-35, FR2 comprises the amino acids at positions 36-49, CDR2 comprises the amino acid residues at positions 50-65, FR3 comprises the amino acid residues at positions 66-94, CDR3 comprises the amino acid residues at positions 95-102, and FR4 comprises the amino acid residues at positions 103-113.

It should be noted, however (as is well known in the art for VH domains and for VHH domains), that the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of VH domains, which methods can also be applied in an analogous manner to VHH domains, are known in the art. However, in the present disclosure, claims and figures, the numbering according to Kabat and applied to VHH domains as described above will be followed, unless indicated otherwise.

In some embodiments, a biparatopic fusion protein described herein includes one or more anti-CLL-1 single domain antibodies. In some embodiments, an anti-CLL-1 single domain antibody is or includes a VHH having the amino acid sequence of any one of SEQ ID Nos:203-225, or a fragment thereof (e.g., a CLL-1 binding fragment thereof). As indicated in the listing of sequences provided herein, each of SEQ ID Nos:203-225 includes VHH amino acids at the N-terminus, and the following amino acids at the C-terminus: (i) a linker of 9 amino acids (TSGPGGQGA), (ii) a myc-tag (EQKLISEEDL), (iii) a linker of 2 amino acids (GA), (iv) a hexa-histidine tag (HIHIHHHH), and (v) an additional 3 amino acids (GAS). Thus, in some embodiments, the disclosure provides biparatopic fusion proteins that include an antibody that is or includes a VHH having a portion (e.g., a CLL-1 binding portion) of the amino acid sequence of any one of SEQ ID Nos:203-225, wherein the portion lacks one or more of (i)-(v) (and/or lacks a portion of one or more of (i)-(v)). In some embodiments, the disclosure provides biparatopic fusion proteins that include an antibody that is or includes a VHH having a portion (e.g., a CLL-1 binding portion) of the amino acid sequence of any one of SEQ ID Nos:203-225, wherein the portion lacks one or more of the C-terminal amino acids TSGPGGQGAEQKLISEEDLGAHHHHHHGAS depicted in each of SEQ ID Nos:203-225. In some embodiments, the disclosure provides biparatopic fusion proteins that include an antibody that is or includes a VHH having a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

In some embodiments, the disclosure provides biparatopic fusion proteins that include an antibody that is or includes a VHH having a portion (e.g., a CLL-1 binding portion) of the amino acid sequence of any one of SEQ ID Nos:203-225, wherein the portion lacks one or more of (i)-(v) (and/or lacks a portion of one or more of (i)-(v)), and wherein the portion lacks one or more (e.g., 1, 2, 3, 4, 5, or more), additional amino acids (i.e., other than an amino acid included in (i)-(v)). In some embodiments, the disclosure provides biparatopic fusion proteins that include an antibody that is or includes a VHH having a portion (e.g., a CLL-1 binding portion) of the amino acid sequence of any one of SEQ ID Nos:203-225, wherein the portion lacks one or more of the C-terminal amino acids TSGPGGQGAEQKLISEEDLGAHHHHHHGAS depicted in each of SEQ ID Nos:203-225, and wherein the portion lacks one or more (e.g., 1, 2, 3, 4, 5, or more), additional amino acids. In some embodiments, the disclosure provides biparatopic fusion proteins that include an antibody that is or includes a VHH having a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225, and wherein the portion lacks one or more (e.g., 1, 2, 3, 4, 5, or more), additional amino acids.

In some embodiments, the disclosure provides biparatopic fusion proteins that include an antibody that is or includes a VHH having an amino acid sequence that is at least 85%, 90%, 91%, 92%, 93%, 94%, 95%, 96%, 97%, 98%, 99%, or 100% identical to a portion (e.g., a CLL-1 binding portion) of the amino acid sequence of any one of SEQ ID Nos:203-225, wherein the portion lacks one or more of (i)-(v) (and/or lacks a portion of one or more of (i)-(v)). In some embodiments, the disclosure provides biparatopic fusion proteins that include an antibody that is or includes a VHH having an amino acid sequence that is at least 85%, 90%, 91%, 92%, 93%, 94%, 95%, 96%, 97%, 98%, 99%, or 100% identical to a portion (e.g., a CLL-1 binding portion) of the amino acid sequence of any one of SEQ ID Nos:203-225, wherein the portion lacks one or more of the C-terminal amino acids TSGPGGQGAEQKLISEEDLGAHHHHHHGAS depicted in each of SEQ ID Nos:203-225. In some embodiments, the disclosure provides biparatopic fusion proteins that include an antibody that is or includes a VHH having an amino acid sequence that is at least 85%, 90%, 91%, 92%, 93%, 94%, 95%, 96%, 97%, 98%, 99%, or 100% identical to a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

In some embodiments, the disclosure provides biparatopic fusion proteins that include an antibody that is or includes a VHH having an amino acid sequence that is at least 85%, 90%, 91%, 92%, 93%, 94%, 95%, 96%, 97%, 98%, 99%, or 100% identical to a portion (e.g., a CLL-1 binding portion) of the amino acid sequence of any one of SEQ ID Nos:203-225, wherein the portion lacks one or more of (i)-(v) (and/or lacks a portion of one or more of (i)-(v)), and wherein the portion lacks one or more (e.g., 1, 2, 3, 4, 5, or more), additional amino acids (i.e., other than an amino acid included in (i)-(v)). In some embodiments, the disclosure provides biparatopic fusion proteins that include an antibody that is or includes a VHH having an amino acid sequence that is at least 85%, 90%, 91%, 92%, 93%, 94%, 95%, 96%, 97%, 98%, 99%, or 100% identical to a portion (e.g., a CLL-1 binding portion) of the amino acid sequence of any one of SEQ ID Nos:203-225, wherein the portion lacks one or more of the C-terminal amino acids TSGPGGQGAEQKLISEEDLGAHHHHHHGAS depicted in each of SEQ ID Nos:203-225, and wherein the portion lacks one or more (e.g., 1, 2, 3, 4, 5, or more), additional amino acids. In some embodiments, the disclosure provides biparatopic fusion proteins that include an antibody that is or includes a VHH having an amino acid sequence that is at least 85%, 90%, 91%, 92%, 93%, 94%, 95%, 96%, 97%, 98%, 99%, or 100% identical to a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225, and wherein the portion lacks one or more (e.g., 1, 2, 3, 4, 5, or more), additional amino acids.

In some embodiments, the disclosure provides biparatopic fusion proteins that include an antibody that is or includes a VHH comprising at least one CDR (e.g., CDR1, CDR2, and/or CDR3) depicted in any one of SEQ ID Nos:203-225. In some embodiments, the disclosure provides biparatopic fusion proteins that include an antibody that is or includes a VHH comprising a portion of at least one CDR (e.g., CDR1, CDR2, and/or CDR3) depicted in any one of SEQ ID Nos:203-225, wherein the portion lacks 1, 2, 3, 4, 5, or more amino acids of a CDR depicted in any one of SEQ ID Nos:203-225. In some embodiments, the disclosure provides biparatopic fusion proteins that include an antibody that is or includes a VHH comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) depicted in any one of SEQ ID Nos:203-225. In some embodiments, the disclosure provides biparatopic fusion proteins that include an antibody that is or includes a VHH comprising an amino acid sequence that is at least 85%, 90%, 91%, 92%, 93%, 94%, 95%, 96%, 97%, 98%, 99%, or 100% identical to a portion of at least one CDR (e.g., CDR1, CDR2, and/or CDR3) depicted in any one of SEQ ID Nos:203-225, wherein the portion lacks 1, 2, 3, 4, 5, or more amino acids of a CDR depicted in any one of SEQ ID Nos:203-225.

In some embodiments, the disclosure provides biparatopic fusion proteins that include an antibody that is or includes a VHH comprising CDR1, CDR2, and/or CDR3 of any one of Groups 1-13 depicted in Table 5A and/or Table 5B. In some embodiments, the disclosure provides biparatopic fusion proteins that include an antibody that is or includes a VHH comprising (i) CDR1 and CDR2; (ii) CDR2 and CDR3; (iii) CDR1 and CDR3; or (iv) CDR1, CDR2, and CDR3 of any one of Groups 1-13 depicted in Table 5A and/or Table 5B (e.g., wherein the CDRs are from one particular Group, or wherein the CDRs are selected from two or more different Groups). Table 5A:

TABLE 5A

| Group | CDR1 | CDR2 | CDR3 |
|---|---|---|---|
| 1 | CAASGSIFAINEI | VAACASDGNTY | DANSRGNYY |
| 2 | CVVSGDTRSI | VAACASDGNTY | DANSRGNYY |
| 3 | CVASGSIRSI | VAACASDGNTY | DANSRGNYY |
| 4 | CAASGFTFNSYA | WVSDINSGGGS | ATELRGSDYY |
| | | TN | RGPIREYAY |
| 5 | CAASGLTFSNYA | AINWSGGTTD | AASYRLRITV |
| | | | VVTPDEYHY |
| 6 | CAASGFAFDDYA | WVSSISWNGG | VKLVDSGWYS |
| | | GTY | AYDY |
| 7 | CVVSGATSNVNA | LVAAISSGGS | AAQDWATEGYE |
| | | TS | YDY |
| 8 | CVVSGTMFSGKD | VATVSSDGGT | HFLWGRHY |
| | | D | |
| 9 | CVASGNDISGSA | VAVDAPRER | GPSLRTFHGRE |
| | | PF | WYRPPWFTS |
| 10 | CAASGSIFSINA | VAVVSRFGET | NARIRGNYGSRI |
| | | T | DY |
| 11 | CVVSGNMLDLNT | LVAALGISTY | ARDYNFES |
| 12 | GSDRSINV | ITSGGTT | KADTRWGGMY |
| 13 | GRTIDNGA | INWSGGAT | ASRRGVDLRR |
| | | | NSYEYDY |

TABLE 5B
CDRs as identified based on IMGT
numbering and ANARCI software
(http://opig.stats.ox.ac.uk/webapps/
sabdab-sabpred/ANARCI.php)

| Group | CDR1 | CDR2 | CDR3 |
|---|---|---|---|
| 1 | GSIFAINEINL | CASDGNT | DANSRGNYY |
| 2 | GDTRSINL | CASDGNT | DANSRGNYY |
| 3 | GSIRSINV | CASDGNT | DANSRGNYY |
| 4 | GFTFNSYA | INSGGGST | ATELRGSDYYRGPIREYAY |
| 5 | GLTFSNYA | INWSGGTT | AASYRLRITVVVTPDEYHY |
| 6 | GFAFDDYA | ISWNGGGT | VKLVDSGWYSAYDY |
| 7 | GATSNVNA | ISSGGST | AAQDWATEGYEYDY |
| 8 | GTMFSGKD | VSSDGGT | HFLWGRHY |
| 9 | GNDISGSA | VDAPRERP | GPSLRTFHGREWYRPPWFTS |
| 10 | GSIFSINA | VSRFGET | NARIRGNYGSRIDY |
| 11 | GNMLDLNT | LGIST | ARDYNFES |
| 12 | GSDRSINV | ITSGGTT | KADTRWGGMY |
| 13 | GRTIDNGA | INWSGGAT | ASRRGVDLRRNSYEYDY |

As will be understood by those of skill in the art, any such CDR sequence may be readily combined, e.g., using molecular biology techniques, with any other antibody sequences or domains provided herein or otherwise known in the art, including any framework regions, CDRs, or constant domains, or portions thereof as disclosed herein or otherwise known in the art, as may be present in an antibody or binding molecule of any format as disclosed herein or otherwise known in the art.

The binding properties of an antibody described herein to an antigen (e.g., CLL-1) can be measured by methods known in the art, e.g., one of the following methods: BIACORE analysis, Enzyme Linked Immunosorbent Assay (ELISA), x-ray crystallography, sequence analysis and scanning mutagenesis. The binding interaction of an antibody and an antigen (e.g., CLL-1) can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Additionally, a KinExA® (Kinetic Exclusion Assay) assay, available from Sapidyne Instruments (Boise, Id.) can also be used.

Information from SPR can be used to provide an accurate and quantitative measure of the equilibrium dissociation constant (K_{D}), and kinetic parameters, including Kₒₙ and K_{off}, for the binding of an antibody to an antigen (e.g., CLL-1). Such data can be used to compare different molecules. Information from SPR can also be used to develop structure-activity relationships (SAR). Variant amino acids at given positions can be identified that correlate with particular binding parameters, e.g., high affinity.

In certain embodiments, an antibody described herein exhibits high affinity for binding an antigen (e.g., CLL-1). In various embodiments, K_{D} of an antibody as described herein for an antigen (e.g., CLL-1) is less than about 10⁻⁴, 10⁻⁵, 10⁻⁶, 10⁻⁷, 10⁻⁹, 10⁻⁹, 10⁻¹⁰, 10⁻¹¹, 10⁻¹², 10⁻¹³, 10⁻¹⁴, or 10⁻¹⁵ M. In certain instances, K_{D} of an antibody as described herein for an antigen (e.g., CLL-1) is between 0.001 and 1 nM, e.g., 0.001 nM, 0.005 nM, 0.01 nM, 0.05 nM, 0.1 nM, 0.5 nM, or 1 nM.

Other Antibodies

Although exemplified biparatopic fusion proteins include antibodies that bind CLL-1, a biparatopic fusion protein can include any antibody that can bind to one or more tumor antigens, including the exemplary antibodies listed in Table 1. Tumor antigens are known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1α, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, and mesothelin.

In some embodiments, a tumor antigen is or comprises one or more antigenic cancer epitopes associated with a malignant tumor. Malignant tumor antigens that include such epitopes include, e.g., tissue-specific antigens such as MART-1, tyrosinase and GP 100 in melanoma and prostatic acid phosphatase (PAP) and prostate-specific antigen (PSA) in prostate cancer. Other tumor antigens belong to the group of transformation-related molecules such as the oncogene HER-2/Neu/ErbB-2. Yet another group of tumor antigens are onco-fetal antigens such as carcinoembryonic antigen (CEA). In B-cell lymphoma the tumor-specific idiotype immunoglobulin constitutes a tumor-specific immunoglobulin antigen that is unique to the individual tumor. B-cell differentiation antigens such as CD19, CD20 and CD37 are other tumor antigens in B-cell lymphoma. Some of these antigens (e.g., CEA, HER-2, CD19, CD20, idiotype) have been used as targets for passive immunotherapy with monoclonal antibodies with limited success.

A tumor antigen described herein can be a tumor-specific antigen (TSA) or a tumor-associated antigen (TAA). A TSA is (or is believed to be) unique to tumor cells and does not occur on other cells in the body (e.g., does not occur to a significant extent on other cells). A TAA is not unique to a tumor cell and instead is also expressed on a normal cell (e.g., expressed under conditions that fail to induce a state of immunologic tolerance to the antigen). For example, TAAs can be antigens that are expressed on normal cells during fetal development when the immune system is immature and unable to respond, or they can be antigens that are normally present at extremely low levels on normal cells but that are expressed at higher levels on tumor cells.

Non-limiting examples of TSA or TAA antigens include differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other tumor antigens include TSP-180, MAGE-4, MAGE-5, MAGE-6, RAGE, NY-ESO, erbB, p185erbB2, p180erbB-3, c-met, nm-23H₁, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68P1, CO-029, FGF-5, G250, Ga733\EpCAM, HTgp-175, M344, MA-50, MG7-Ag, MOV18, NB/70K, NY-CO-1, RCAS1, SDCCAG16, TA-90Mac-2 binding protein\cyclophilin C-associated protein, TAAL6, TAG72, TLP, MUC16, IL13Rα2, FRα, VEGFR2, Lewis Y, FAP, EphA2, CEACAM5, EGFR, CA6, CA9, GPNMB, EGP1, FOLR1, endothelial receptor, STEAP1, SLC44A4, Nectin-4, AGS-16, guanylyl cyclase C, MUC-1, CFC1B, integrin alpha 3 chain (of a3b1, a laminin receptor chain), and TPS.

In some embodiments, a tumor antigen is CD19, CD20, CD22, CD30, CD72, CD180, CD171 (L1CAM), CD123, CD133, CD138, CD37, CD70, CD79a, CD79b, CD56, CD74, CD166, CD71, CLL-1/CLECK12A, ROR1, Glypican 3 (GPC3), Mesothelin, CD33/IL3Ra, IL1RAP, c-Met, PSCA, PSMA, Glycolipid F77, EGFRvIII, GD-2, MY-ESO-1, or MAGE A3. Additional tumor antigens can be identified, e.g., by sequencing tumor genomes and exomes, and/or by high-sensitivity mass spectrometry analysis of the tumor proteome, any of which can be used in methods described herein. Other tumor antigens are described in, e.g., WO2017/075537.

In some embodiments, a tumor antigen is a generic or “housekeeping” membrane protein, e.g., found on every cell. In some embodiments, a tumor antigen is a tumor stem cell marker. In some embodiments, a tumor antigen is a neoantigen (i.e., an antigen that arises in a tumor itself, e.g., because of aberrant proliferation).

Biparatopic Fusion Proteins with Cleavable Linkers

In some embodiments, any of the biparatopic fusion proteins described herein can include a linker between any of the components of the fusion protein (e.g., scFv, VHH, CD19 variant). A variety of suitable linkers and methods for preparing fusion proteins including linkers are known in the art. The linker can be cleavable, e.g., under physiological conditions, e.g., under intracellular conditions, such that cleavage of the linker releases the fusion partners. The linker can be, e.g., a peptidyl linker that is cleaved by, e.g., a plasma peptidase or protease enzyme, including, but not limited to, aminopeptidase, plasmin, and kinin-kallikrein. In some embodiments, the linker can be cleaved by a tumor associated protease, e.g., matriptase, Cathepsin B. In some embodiments, cleavage by a tumor-associated protease induces a conformational change in CD19 allowing for binding and/or expression of the CAR epitope to allow killing. In some embodiments, the peptidyl linker is at least two amino acids long or at least three amino acids long. In some embodiments, the peptidyl linker is P2A.

Masked Biparatopic Fusion Proteins

In some embodiments, an expressed biparatopic fusion protein is or includes a masked version of one or more antigen-binding protein(s) described herein (e.g., antibody or antibody fragment described herein). In some embodiments, an expressed biparatopic fusion protein includes a masked version of an antibody or antibody fragment described herein (e.g., a Probody® as described in, e.g., Sandersjoo et al. Cell. Mol. Life Sci. (2015) 72:1405-1415; US 2015/0183875; U.S. Pat. Nos. 8,513,390; and 9,120,853). In some embodiments, a masked fusion protein comprises an antibody, or fragment thereof, a masking moiety, a cleavable moiety, and/or a linker.

In some embodiments, a masked fusion protein comprises two or more antigen-binding proteins (e.g., antibody, or fragment thereof), and a masking moiety. In some embodiments, a masking moiety is an amino acid sequence coupled to the antigen-binding protein (e.g., antibody or fragment), and positioned such that it reduces the protein's ability to specifically bind its target (“masking” the antigen-binding protein). In some embodiments, a masking moiety is coupled to the antigen-binding protein by way of a linker. In some embodiments, specific binding of a masked antigen-binding protein, to its target is reduced or inhibited, as compared to the specific binding of an “unmasked” antigen-binding protein, or as compared to the specific binding of the parental antigen-binding protein, to the target. In some embodiments, a masked antigen-binding protein demonstrates no measurable binding or substantially no measurable binding to the target, and/or demonstrates no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding to the target, as compared to the binding of an unmasked antigen-binding protein, or as compared to the binding of the parental antigen-binding protein to the target, e.g., for at least 2, 4, 6, 8, 12, 28, 24, 30, 36, 48, 60, 72, 84, 96 hours, or 5, 10, 15, 30, 45, 60, 90, 120, 150, 180 days, or 1, 2, 3, 4, 5, 6, 7, 8, 9, 10, 11, 12 months or greater, e.g., when measured in vivo or in a Target Displacement in vitro immunoabsorbent assay (described in U.S. Pat. No. 8,513,390).

In some embodiments, specific binding of a masked antigen-binding protein to its target is reduced or inhibited, as compared to specific binding of the unmasked antigen-binding protein, or as compared to the specific binding of the parental antigen-binding protein to the target. The K_{d} of the masked antigen-binding protein towards the target can be at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times greater than that of the unmasked antigen-binding protein, or than that of the parental antigen-binding protein. Conversely, the binding affinity of the masked antigen-binding protein towards the target can be at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times lower than that of the unmasked antigen-binding protein, or than that of the parental antigen-binding protein.

Masking moieties are known in the art and include, e.g., known binding partners of antibodies, or fragments thereof. In some embodiments, a masking moiety is an amino acid sequence at the N-terminus, at the C-terminus, and/or within an internal site (e.g., an antigen binding loop) of the antigen-binding protein. In some embodiments, a masking moiety is or includes one or more pairs of cysteine residues, e.g., resulting in formation of a disulfide bond between cysteine pairs. In some such embodiments, disulfide bonds result in a conformationally constrained structure, which can be “unmasked” by cleavage of the disulfide bond by, e.g., a reducing agent. Exemplary masking moieties are described in, e.g., Sandersjoo et al. Cell. Mol. Life Sci. (2015) 72:1405-1415; US 2015/0183875; U.S. Pat. Nos. 8,513,390; and 9,120,853.

In some embodiments, a masked biparatopic fusion protein includes a masking moiety on one or more of the antigen binding proteins. In some embodiments, a masking moiety is at the N-terminus of one or more antigen binding proteins included in an expressed biparatopic fusion protein. In some embodiments, a masking moiety is at the C-terminus of one or more antigen binding proteins included in an expressed biparatopic fusion protein. In some embodiments, a masked antibody additionally includes one or more cleavable moieties. In some embodiments, a cleavable moiety is or includes, e.g., one or more amino acid sequences that can serve as a substrate for one or more proteases, such as one or more extracellular proteases. In some embodiments, a cleavable moiety is or includes a cysteine-cysteine pair capable of forming a disulfide bond, which can be cleaved by action of a reducing agent. In other embodiments, a cleavable moiety is or includes a substrate capable of being cleaved upon photolysis.

In some embodiments, a cleavable moiety is selected based on presence of a protease in or in proximity to tissue with a desired target of an antibody, or fragment thereof. In some embodiments, target tissue is a cancerous tissue. Proteases having substrates in a number of cancers, e.g., solid tumors, are known in the art (see, e.g., La Rocca et al, (2004) British J. of Cancer 90(7): 1414-1421). In some embodiments, a cleavable moiety is or includes a target for, e.g., legumain, plasmin, TMPRSS-3/4, MMP-9, MT1-MMP, ADAM (a disintegrin and metalloproteinase, e.g., ADAMs1-20, e.g., ADAM8, ADAM10, ADAM17), cathepsin (e.g., cathepsin A, B, C, D, E, F, G, H, L, K, O, S, V, or W (Tan et al., World J. Biol. Chem. 4:91-101 (2013)), caspase, human neutrophil elastase, beta-secretase, matriptase, uPA, or PSA.

In some embodiments, a masked fusion protein described herein includes a linker, e.g., C-terminal and/or N-terminal to a masking moiety and/or cleavage moiety. In some embodiments, a linker may provide flexibility for the masking moiety to reversibly inhibit binding of the antigen-binding protein to its target. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids. In some embodiments, a masking moiety is fused to an antigen-binding protein through a polypeptide linker. In some embodiments, a linker used to fuse a masking moiety to an antigen-binding protein is a cleavable moiety described herein. In some embodiments a masking moiety is fused, directly or by linker, to the N-terminus of an antigen-binding protein. In some embodiments a masking moiety is fused, directly or by linker, to the C-terminus of an antigen-binding protein.

In some embodiments, a masked fusion protein described herein can additionally or alternatively be produced and/or purified using known methods. In some embodiments, such produced and/or purified masked fusion protein can be used, as described herein, as a protein therapeutic.

### Methods of Producing Cellular Therapeutics

In general, a cellular therapeutic described herein can be produced from an immune cell, e.g., a cell useful in or capable of use in adoptive cell therapy. In some embodiments, a cellular therapeutic is produced from a cell type selected from a group consisting of TILs, T-cells, virus specific T cells (VSTs), CD8⁺ cells, CD4⁺ cells, NK-cells, delta-gamma T-cells, regulatory T-cells, peripheral blood mononuclear cells or IPSC derived cells. As used herein “tumor-infiltrating lymphocytes” or TILs refer to white blood cells that have left the bloodstream and migrated into a tumor. Lymphocytes can be divided into three groups including B cells, T cells and natural killer cells. As used herein “T-cells” refers to CD3⁺ cells, including CD4⁺ helper cells, CD8⁺ cytotoxic T-cells and delta-gamma T cells.

In certain embodiments a cellular therapeutic is produced by genetically modifying (e.g., transforming) a cell, e.g., an immune cell, with a nucleic acid encoding an antigen binding receptor and/or an expression construct described herein (e.g., (i) a first recombinant expression vector that includes a nucleic acid encoding an antigen binding receptor and a second recombinant expression vector that includes an inducible expression construct, (ii) a single recombinant expression vector that includes both a nucleic acid encoding an antigen binding receptor and an inducible expression construct; or (iii) a recombinant expression vector that includes a constitutive expression construct). The recombinant expression vector can comprise any type of nucleotides, including, but not limited to DNA and RNA, which can be single-stranded or double-stranded, synthesized or obtained in part from natural sources, and which can contain natural, non-natural or altered nucleotides. A recombinant expression vector can comprise naturally-occurring or non-naturally-occurring internucleotide linkages, or both types of linkages. In some embodiments, a recombinant expression vector can be or comprise a transposon.

A recombinant expression vector can be any suitable recombinant expression vector. Suitable vectors include those designed for propagation and expansion or for expression or both, such as plasmids and viruses. For example, a vector can be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, also can be used. Examples of plant expression vectors useful in the context of the disclosure include pBI01, pBI101.2, pBI101.3, pBI121 and pBIN19 (Clontech). Examples of animal expression vectors useful in the context of the disclosure include pcDNA, pEUK-Cl, pMAM, and pMAMneo (Clontech). In some embodiments, a bicistronic IRES vector (e.g., from Clontech) is used to include both a nucleic acid encoding an antigen binding receptor and an inducible expression construct described herein.

In some embodiments, a recombinant expression vector is a viral vector. Suitable viral vectors include, without limitation, retroviral vectors, alphaviral, vaccinial, adenoviral, adeno-associated viral, herpes viral, and fowl pox viral vectors, and preferably have a native or engineered capacity to transform an immune cell (e.g., T cell).

Recombinant expression vectors can be prepared using standard recombinant DNA or RNA techniques described in, for example, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, N Y, 1994. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

A recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the recombinant expression vectors include, for instance, neomycin/G418 resistance genes, puromycin resistance genes, hygromycin resistance genes, histidinol resistance genes, tetracycline resistance genes, and ampicillin resistance genes.

Vectors useful in the context of the disclosure can be “naked” nucleic acid vectors (i.e., vectors having little or no proteins, sugars, and/or lipids encapsulating them), or vectors complexed with other molecules. Other molecules that can be suitably combined with the vectors include without limitation viral coats, cationic lipids, liposomes, polyamines, gold particles, and targeting moieties such as ligands, receptors, or antibodies that target cellular molecules.

Vector DNA or RNA can be introduced into a cell, e.g., an immune cell, via conventional transformation or transfection techniques. As used herein, the terms “transformation” and “transfection” are intended to refer to a variety of art-recognized techniques for introducing foreign nucleic acid (e.g., DNA or RNA) into a cell, including calcium phosphate or calcium chloride co-precipitation, DEAE-dextran-mediated transfection, lipofection, gene gun, or electroporation.

### Protein Therapeutics

In some aspects, biparatopic fusion proteins described herein can be produced and used as protein therapeutics instead of, or in addition to, being produced by a cell (e.g., cellular therapeutic) described herein. Such polypeptides can be included in a composition, e.g., a pharmaceutical composition, and used as a protein therapeutic. For example, a protein therapeutic that includes a polypeptide that is or comprises a target for a cellular therapeutic, e.g., a CAR-T cell or ADC, can be administered in combination with such cellular therapeutic, e.g., CAR-T cell or ADC.

In one example, a protein therapeutic includes a biparatopic fusion protein that includes a first antibody (e.g., anti-CLL-1 antibody, e.g., anti-CLL-1 scFv, e.g., anti-CLL-1 scFv described herein), a second antibody (e.g., anti-CLL-1 antibody, e.g., anti-CLL-1 VHH, e.g., anti-CLL-1 VHH described herein), and a CD19 variant described herein.

A variety of methods of making polypeptides are known in the art and can be used to make a polypeptide to be included in a protein therapeutic. For example, a polypeptide can be recombinantly produced by utilizing a host cell system engineered to express a nucleic acid encoding the polypeptide. Recombinant expression of a gene can include construction of an expression vector containing a polynucleotide that encodes the polypeptide. Once a polynucleotide has been obtained, a vector for the production of the polypeptide can be produced by recombinant DNA or RNA technology using techniques known in the art. Known methods can be used to construct expression vectors containing polypeptide coding sequences and appropriate transcriptional and translational control signals. These methods include, for example, in vitro recombinant DNA or RNA techniques, synthetic techniques, and in vivo genetic recombination.

An expression vector can be transferred to a host cell by conventional techniques, and transfected cells can then be cultured by conventional techniques to produce polypeptide.

A variety of host expression vector systems can be used (see, e.g., U.S. Pat. No. 5,807,715). Such host-expression systems can be used to produce polypeptides and, where desired, subsequently purified. Such host expression systems include microorganisms such as bacteria (e.g., E. coli and B. subtilis) transformed with recombinant bacteriophage DNA, plasmid DNA or cosmid DNA expression vectors containing polypeptide coding sequences; yeast (e.g., Saccharomyces and Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing polypeptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For bacterial systems, a number of expression vectors can be used, including, but not limited to, the E. coli expression vector pUR278 (Ruther et al., 1983, EMBO 12:1791); pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST).

For expression in mammalian host cells, viral-based expression systems can be utilized (see, e.g., Logan & Shenk, 1984, Proc. Natl. Acad. Sci. USA 8 1:355-359). The efficiency of expression can be enhanced by inclusion of appropriate transcription enhancer elements, transcription terminators, etc. (see, e.g., Bittner et al., 1987, Methods in Enzymol. 153:516-544).

In addition, a host cell strain can be chosen that modulates expression of inserted sequences, or modifies and processes the gene product in the specific fashion desired. Different host cells have characteristic and specific mechanisms for post-translational processing and modification of proteins and gene products. Appropriate cell lines or host systems can be chosen to ensure the correct modification and processing of the polypeptide expressed. Such cells include, for example, established mammalian cell lines and insect cell lines, animal cells, fungal cells, and yeast cells. Mammalian host cells include, e.g., BALB/c mouse myeloma line (NSO/l, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); human fibrosarcoma cell line (e.g., HT1080); baby hamster kidney cells (BHK, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

For long-term, high-yield production of recombinant proteins, host cells are engineered to stably express a polypeptide. Host cells can be transformed with DNA controlled by appropriate expression control elements known in the art, including promoter, enhancer, sequences, transcription terminators, polyadenylation sites, and selectable markers. Methods commonly known in the art of recombinant DNA technology can be used to select a desired recombinant clone.

Once a polypeptide and/or fusion protein described herein has been produced by recombinant expression, it may be purified by any method known in the art for purification, for example, by chromatography (e.g., ion exchange, affinity, and sizing column chromatography), centrifugation, differential solubility, or by any other standard technique for purification of proteins. For example, an antibody can be isolated and purified by appropriately selecting and combining affinity columns such as Protein A column with chromatography columns, filtration, ultra filtration, salting-out and dialysis procedures (see Antibodies: A Laboratory Manual, Ed Harlow, David Lane, Cold Spring Harbor Laboratory, 1988). Further, as described herein, a polypeptide and/or fusion protein can be fused to heterologous polypeptide sequences to facilitate purification. Alternatively or additionally, a polypeptide and/or fusion protein can be partially or fully prepared by chemical synthesis. For example, polypeptides included in a biparatopic fusion protein described herein can be produced (e.g., recombinantly and/or chemically synthesized) and conjugated (e.g., chemically conjugated) to produce the fusion protein. Alternatively or additionally, a polypeptide can be purified from natural sources.

### Viral Delivery

In some embodiments, a nucleic acid encoding biparatopic fusion protein described can be introduced in a viral vector. In some embodiments, such a viral vector can be used to introduce a biparatopic fusion protein into a cancer cell (e.g., a tumor cell). Introduction of such biparatopic fusion protein can increase susceptibility to a subject's immune system and/or one or more additional therapeutic agents (see, e.g., WO2017/075533).

Vector Design

A nucleic acid sequence encoding a biparatopic fusion protein described herein can be cloned into a number of types of vectors. For example, a nucleic acid can be cloned into a plasmid, a phagemid, a phage derivative, an animal virus, and a cosmid. Other vectors can include expression vectors, replication vectors, probe generation vectors, sequencing vectors, and viral vectors. In other examples, the vector can be a foamy viral (FV) vector, a type of retroviral vector made from spumavirus. Viral vector design and technology is well known in the art as described in Sambrook et al, (Molecular Cloning: A Laboratory Manual, 2001), and in other virology and molecular biology manuals.

Viral Transduction

Viruses are highly efficient at nucleic acid delivery to specific cell types, while often avoiding detection by the infected host immune system. These features make certain viruses attractive candidates as vehicles for introduction of cellular therapy targets into cancer cells, e.g., solid tumor cells. A number of viral based systems have been developed for gene transfer into mammalian cells. Examples of viral vectors include, but are not limited to, retroviruses, adenoviruses, adeno-associated viruses, herpes viruses, lentiviruses, poxviruses, herpes simplex 1 virus, herpes virus, oncoviruses (e.g., murine leukemia viruses), and the like. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Lentiviral and Retroviral transduction can be enhanced by the addition of polybrene (SantaCruz sc-134220; Millipore TR-1003-G; Sigma 107689), a cationic polymer (also known as hexamehtrine bromide) that is used to increase the efficiency of the retrovirus transduction.

For example, retroviruses provide a platform for gene delivery systems. Retroviruses are enveloped viruses that belong to the viral family Retroviridae. Once in a host's cell, the virus replicates by using a viral reverse transcriptase enzyme to transcribe its RNA into DNA. The retroviral DNA replicates as part of the host genome, and is referred to as a provirus. A selected gene can be inserted into a vector and packaged in retroviral particles using techniques known in the art. The recombinant virus can then be isolated and delivered to cells of the subject in vivo. A number of retroviral systems are known in the art, (see, e.g., U.S. Pat. Nos. 5,994,136, 6,165,782, and 6,428,953).

Retroviruses include the genus of Alpharetrovirus (e.g., avian leukosis virus), the genus of Betaretrovirus; (e.g., mouse mammary tumor virus) the genus of Deltaretrovirus (e.g., bovine leukemia virus and human T-lymphotropic virus), the genus of Epsilonretrovirus (e.g., Walleye dermal sarcoma virus), and the genus of Lentivirus. In some embodiments, a retrovirus is a lentivirus a genus of viruses of the Retroviridae family, e.g., characterized by a long incubation period. Lentiviruses are unique among the retroviruses in being able to infect non-dividing cells; they can deliver a significant amount of genetic information into the DNA of the host cell, so can be used as an efficient gene delivery vector. In some examples, a lentivirus can be, but not limited to, human immunodeficiency viruses (HIV-1 and HIV-2), simian immunodeficiency virus (S1V), feline immunodeficiency virus (FIV), equine infections anemia (EIA), and visna virus. Vectors derived from lentiviruses offer the means to achieve significant levels of gene transfer in vivo.

In some embodiments, a vector is an adenovirus vector. Adenoviruses are a large family of viruses containing double stranded DNA. They replicate the DNA of the host cell, while using a host's cell machinery to synthesize viral RNA DNA and proteins. Adenoviruses are known in the art to affect both replicating and non-replicating cells, to accommodate large transgenes, and to code for proteins without integrating into the host cell genome.

In some embodiments, an AAVP vector is used. An AAVP vector is a hybrid of prokaryotic-eukaryotic vectors, which are chimeras of genetic cis-elements of recombinant adeno-associated virus and phage. An AAVP combines selected elements of both phage and AAV vector systems, providing a vector that is simple to produce in bacteria and can exhibit little or no packaging limit, while allowing infection of mammalian cells combined with integration into the host chromosome. Vectors containing many of the appropriate elements are commercially available, and can be further modified by standard methodologies to include the necessary sequences. Among other things, AAVPs do not require helper viruses or trans-acting factors. In addition, the native tropism of AAV for mammalian cells is eliminated since there is not AAV capsid formation. Other methods and details are in U.S. Pat. No. 8,470,528 and Hajitou A. et al., Cell, 125: 358-398.

In some embodiments, a human papilloma (HPV) pseudovirus is used. DNA plasmids can be packaged into papillomavirus L1 and L2 capsid protein to generate pseudovirion that can efficiently deliver DNA. The encapsulation can protect the DNA from nucleases and provides a targeted delivery with a high level of stability. Many of the safety concerns associated with the use of viral vectors can be mitigated with an HPV pseudovirus. Other methods and examples are in Hung, C., et al., Plos One, 7:7 (e40983); 2012, U.S. Pat. No. 8,394,411, and Kines, R., et al Int J of Cancer, 2015.

In some embodiments, an oncolytic virus is used. Oncolytic virus therapy can selectively replicate the virus in cancer cells, and can subsequently spread within a tumor, e.g., without affecting normal tissue. Alternatively, an oncolytic virus can preferentially infect and kill cells without causing damage to normal tissues. Oncolytic viruses can also effectively induce immune responses to themselves as well as to the infected tumor cell. Typically, oncolytic viruses fall into two classes: (I) viruses that naturally replicate preferentially in cancer cells and are nonpathogenic in humans. Exemplary class (I) oncolytic viruses include autonomous parvoviruses, myxoma virus (poxvirus), Newcastle disease virus (NDV; paramyxovirus), reovirus, and Seneca valley virus (picornavirus). A second class (II), includes viruses that are genetically manipulated for use as vaccine vectors, including measles virus (paramyxovirus), poliovirus (picornavirus), and vaccinia virus (poxvirus). Additionally, oncolytic viruses may include those genetically engineered with mutations/deletions in genes required for replication in normal but not in cancer cells including adenovirus, herpes simplex virus, and vesicular stomatitis virus. Oncolytic viruses can be used as a viral transduction method due to their low probability of genetic resistance because they can target multiple pathways and replicate in a tumor-selective method. The viral dose within a tumor can increase over time due to in situ viral amplification (as compared to small molecule therapies which decrease with time), and safety features can be built in (i.e., drug and immune sensitivity).

### Administration

Certain embodiments of the disclosure include methods of administering to a subject a cellular therapeutic described herein (or a population thereof), a protein therapeutic described herein, a composition comprising a cellular therapeutic, and/or a composition comprising a protein therapeutic, e.g., in an amount effective to treat a subject. In some embodiments, the method effectively treats cancer in the subject.

In some embodiments, an immune cell is obtained from a subject and is transformed, e.g., transduced, with inducible expression construct or a constitutive expression construct described herein, e.g., an expression vector comprising an inducible expression construct or a constitutive expression construct described herein, to obtain a cellular therapeutic. Thus, in some embodiments, a cellular therapeutic comprises an autologous cell that is administered into the same subject from which an immune cell was obtained. Alternatively, an immune cell is obtained from a subject and is transformed, e.g., transduced, with an inducible expression construct or a constitutive expression construct described herein, e.g., an expression vector comprising an inducible expression construct or a constitutive expression construct described herein, to obtain a cellular therapeutic that is allogenically transferred into another subject.

In some embodiments, a cellular therapeutic is autologous to a subject, and the subject can be immunologically naive, immunized, diseased, or in another condition prior to isolation of an immune cell from the subject.

In some embodiments, additional steps can be performed prior to administration to a subject. For instance, a cellular therapeutic can be expanded in vitro after contacting (e.g., transducing or transfecting) an immune cell with an inducible expression construct or a constitutive expression construct described herein (e.g., an expression vector comprising an inducible expression construct or a constitutive expression construct), but prior to the administration to a subject. In vitro expansion can proceed for 1 day or more, e.g., 2 days or more, 3 days or more, 4 days or more, 6 days or more, or 8 days or more, prior to the administration to a subject. Alternatively, or in addition, in vitro expansion can proceed for 21 days or less, e.g., 18 days or less, 16 days or less, 14 days or less, 10 days or less, 7 days or less, or 5 days or less, prior to administration to a subject. For example, in vitro expansion can proceed for 1-7 days, 2-10 days, 3-5 days, or 8-14 days prior to the administration to a subject.

In some embodiments, during in vitro expansion, a cellular therapeutic can be stimulated with an antigen (e.g., a TCR antigen). Antigen specific expansion optionally can be supplemented with expansion under conditions that non-specifically stimulate lymphocyte proliferation such as, for example, anti-CD3 antibody, anti-Tac antibody, anti-CD28 antibody, or phytohemagglutinin (PHA). The expanded cellular therapeutic can be directly administered into a subject or can be frozen for future use, i.e., for subsequent administrations to a subject.

In some embodiments, a cellular therapeutic is treated ex vivo with interleukin-2 (IL-2) prior to infusion into a cancer patient, and the cancer patient is treated with IL-2 after infusion. In some embodiments, a cellular therapeutic is treated ex vivo with IL-2 and/or other cytokines, eg., IL-7, IL-15 and/or IL-21. Furthermore, in some embodiments, a cancer patient can undergo preparative lymphodepletion—the temporary ablation of the immune system—prior to administration of a cellular therapeutic. A combination of cytokine treatment and preparative lymphodepletion can enhance persistence of a cellular therapeutic.

In some embodiments, a cellular therapeutic is transduced or transfected with a nucleic acid encoding a cytokine, which nucleic acid can be engineered to provide for constitutive, regulatable, or temporally-controlled expression of the cytokine. Suitable cytokines include, for example, cytokines which act to enhance the survival of T lymphocytes during the contraction phase, which can facilitate the formation and survival of memory T lymphocytes.

In certain embodiments, a cellular therapeutic is administered prior to, substantially simultaneously with, or after the administration of another therapeutic agent, such as a cancer therapeutic agent. The cancer therapeutic agent can be, e.g., a chemotherapeutic agent, a biological agent, or radiation treatment. In some embodiments, a subject receiving a cellular therapeutic is not administered a treatment which is sufficient to cause a depletion of immune cells, such as lymphodepleting chemotherapy or radiation therapy.

A cellular therapeutic described herein can be formed as a composition, e.g., a cellular therapeutic and a pharmaceutically acceptable carrier. In certain embodiments, a composition is a pharmaceutical composition comprising at least one cellular therapeutic described herein and a pharmaceutically acceptable carrier, diluent, and/or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., a cellular therapeutic, and does not elicit any detrimental side effects or toxicity under the conditions of use.

A composition can be formulated for administration by any suitable route, such as, for example, intravenous, intratumoral, intraarterial, intramuscular, intraperitoneal, intrathecal, epidural, and/or subcutaneous administration routes. Preferably, the composition is formulated for a parenteral route of administration.

A composition suitable for parenteral administration can be an aqueous or nonaqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

Dosage administered to a subject, particularly a human, will vary with the particular embodiment, the composition employed, the method of administration, and the particular site and subject being treated. However, a dose should be sufficient to provide a therapeutic response. A clinician skilled in the art can determine the therapeutically effective amount of a composition to be administered to a human or other subject in order to treat or prevent a particular medical condition. The precise amount of the composition required to be therapeutically effective will depend upon numerous factors, e.g., such as the specific activity of the cellular therapeutic, and the route of administration, in addition to many subject-specific considerations, which are within those of skill in the art.

Any suitable number cellular therapeutic cells can be administered to a subject. While a single cellular therapeutic cell described herein is capable of expanding and providing a therapeutic benefit, in some embodiments, 10² or more, e.g., 10³ or more, 10⁴ or more, 10⁵ or more, or 10⁸ or more, cellular therapeutic cells are administered. Alternatively, or additionally 10¹² or less, e.g., 10¹¹ or less, 10⁹ or less, 10⁷ or less, or 10⁵ or less, cellular therapeutic cells described herein are administered to a subject. In some embodiments, 10²-10⁵, 10⁴-10⁷, 10³-10⁹, or 10⁵-10¹⁰ cellular therapeutic cells described herein are administered.

A dose of a cellular therapeutic described herein can be administered to a mammal at one time or in a series of subdoses administered over a suitable period of time, e.g., on a daily, semi-weekly, weekly, bi-weekly, semi-monthly, bi-monthly, semi-annual, or annual basis, as needed. A dosage unit comprising an effective amount of a cellular therapeutic may be administered in a single daily dose, or the total daily dosage may be administered in two, three, four, or more divided doses administered daily, as needed.

A polypeptide described herein can be incorporated into a pharmaceutical composition (e.g., for use as a protein therapeutic). Pharmaceutical compositions comprising a polypeptide can be formulated by methods known to those skilled in the art (see, e.g., Remington's Pharmaceutical Sciences pp. 1447-1676 (Alfonso R. Gennaro, ed., 19th ed. 1995)). Pharmaceutical composition can be administered parenterally in the form of an injectable formulation comprising a sterile solution or suspension in water or another pharmaceutically acceptable liquid. For example, a pharmaceutical composition can be formulated by suitably combining a polypeptide with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in pharmaceutical preparations is such that a suitable dose within the designated range is provided.

The sterile composition for injection can be formulated in accordance with conventional pharmaceutical practices using distilled water for injection as a vehicle. For example, physiological saline or an isotonic solution containing glucose and other supplements such as D-sorbitol, D-mannose, D-mannitol, propylene glycol, polyethylene glycol and sodium chloride may be used as an aqueous solution for injection.

Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

Route of administration can be parenteral, for example, administration by injection, transnasal administration, transpulmonary administration, or transcutaneous administration. Administration can be systemic or local by intravenous injection, intramuscular injection, intraperitoneal injection, subcutaneous injection.

A suitable means of administration can be selected based on the age and condition of the subject. A single dose of a pharmaceutical composition containing a polypeptide can be selected from a range of 0.001 to 1,000 mg/kg of body weight. On the other hand, a dose can be selected in the range of 0.001 to 1,00000 mg/body weight, but the present disclosure is not limited to such ranges. Dose and method of administration can vary depending on the weight, age, condition, and the like of the subject, and can be suitably selected as needed by those skilled in the art.

### Tumors

The present disclosure provides technologies useful in the treatment of any tumor. In some embodiments, a tumor is or comprises a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms. In some embodiments, a tumor is a melanoma. In some embodiments, a tumor is a B cell tumor.

In some embodiments, a tumor is or comprises a solid tumor, including but not limited to breast carcinoma, a squamous cell carcinoma, a colon cancer (e.g., colorectal), a head and neck cancer, ovarian cancer, a lung cancer, mesothelioma, a genitourinary cancer, a rectal cancer, a gastric cancer, or an esophageal cancer.

In some particular embodiments, a tumor is or comprises an advanced tumor, and/or a refractory tumor. In some embodiments, a tumor is characterized as advanced when certain pathologies are observed in a tumor (e.g., in a tissue sample, such as a biopsy sample, obtained from a tumor) and/or when cancer patients with such tumors are typically considered not to be candidates for conventional chemotherapy. In some embodiments, pathologies characterizing tumors as advanced can include tumor size, altered expression of genetic markers, invasion of adjacent organs and/or lymph nodes by tumor cells. In some embodiments, a tumor is characterized as refractory when patients having such a tumor are resistant to one or more known therapeutic modalities (e.g., one or more conventional chemotherapy regimens) and/or when a particular patient has demonstrated resistance (e.g., lack of responsiveness) to one or more such known therapeutic modalities.

### Combination Therapy

As described herein, in some embodiments, a cellular therapeutic and/or a protein therapeutic is administered in combination with a second cellular therapeutic, an antibody-drug conjugate, an antibody, and/or a polypeptide. In some embodiments, the extent of tumor targeting and/or killing by a second cellular therapeutic (e.g., CAR-T cell) is higher than a level observed or measured in the absence of combined therapy with a cellular therapeutic or a protein therapeutic described herein.

A pharmaceutical composition comprising a cellular therapeutic and/or a protein therapeutic described herein can optionally contain, and/or be administered in combination with, one or more additional therapeutic agents, such as a cancer therapeutic agent, e.g., a chemotherapeutic agent or a biological agent. Examples of chemotherapeutic agents that can be used in combination with a cellular therapeutic described herein include platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, pemetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicalutamide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

Examples of biological agents that can be used in the compositions and methods described herein include monoclonal antibodies (e.g., rituximab, cetuximab, panitumumab, tositumomab, trastuzumab, alemtuzumab, gemtuzumab ozogamicin, bevacizumab, catumaxomab, denosumab, obinutuzumab, ofatumumab, ramucirumab, pertuzumab, ipilimumab, nivolumab, nimotuzumab, lambrolizumab, pidilizumab, siltuximab, BMS-936559, RG7446/MPDL3280A, MEDI4736, tremelimumab, or others listed in Table 1 herein), enzymes (e.g., L-asparaginase), cytokines (e.g., interferons and interleukins), growth factors (e.g., colony stimulating factors and erythropoietin), cancer vaccines, gene therapy vectors, or any combination thereof.

In some embodiments, treatment methods described herein are performed on subjects for which other treatments of the medical condition have failed or have had less success in treatment through other means. Additionally, the treatment methods described herein can be performed in conjunction with one or more additional treatments of the medical condition. For instance, the method can comprise administering a cancer regimen, e.g., nonmyeloablative chemotherapy, surgery, hormone therapy, and/or radiation, prior to, substantially simultaneously with, or after the administration of a cellular therapeutic and/or a protein therapeutic described herein, or composition thereof. In certain embodiments, a subject to which a cellular therapeutic and/or a protein therapeutic described herein is administered can also be treated with antibiotics and/or one or more additional pharmaceutical agents.

Exemplary amino acid and nucleotide sequences of the disclosure are listed in the following Table:

TABLE 7

| | Amino acid | Nucleotide | |
|---|---|---|---|
| Construct | SEQ ID | SEQ ID | |
| No: | NO: | NO: | Name |
| 186 | 301 | 300 | CD19-anti-CLL-1 scFv |
| 321 | 303 | 302 | CD 19 variant-anti-CLL-1 |
| | | | VHH(1B12) |
| 330 | 305 | 304 | anti-CLL-1 VHH(2H3)-CD19 |
| | | | variant |
| 357 | 307 | 306 | anti-CLL-1 scFv-anti-CLL-1 |
| | | | VHH(2H3)-CD19 variant |
| 221 | 309 | 308 | CAR-CD19 that secretes |
| | | | the fusion protein #186 |
| 405 | 311 | 310 | CAR-CD19 that secretes |
| | | | the fusion protein #357 |
| 355 | 313 | 312 | CAR-CD19 that secretes |
| | | | the fusion protein #321 |
| 356 | 315 | 314 | CAR-CD19 that secretes |
| | | | the fusion protein #330 |
| 142 | 317 | 316 | CAR-CD19 that secretes a |
| | | | CD19-anti-Her2 scFv |
| 260 | 319 | 318 | CAR-CLL-1scFv |
| 440 | 321 | 320 | anti-CD33 scFv-anti-CLL VHH |
| | | | (2H3)- CD 19 variant |
| 518 | 323 | 322 | anti-CLL-1 scFv-anti-CLL-1 |
| | | | VHH(2H3)-CD19 variant |
| | | | (no His-tag) |
| 410 | 325 | 324 | anti-CD33 scFv-variant |
| | | | CD19-His |
| 468 | 327 | 326 | CAR-CD19 without a Flag |
| | | | tag that secretes the fusion |
| | | | protein #518 |
| 408 | 331 | 330 | CAR anti-CLL-1 VHH (2H3) |

In any of the embodiments described herein, a protein and/or fusion protein described herein has an amino acid sequence having at least 80%, 85%, 90%, 91%, 92%, 93%, 94%, 95%, 96%, 97%, 98%, or 99% identity to a disclosed amino acid sequence, and/or is encoded by a nucleotide sequence having at least 80%, 85%, 90%, 91%, 92%, 93%, 94%, 95%, 96%, 97%, 98%, or 99% identity to a disclosed nucleotide sequence herein.

All publications, including GenBank sequences, cited herein are expressly incorporated by reference herein.

### EXEMPLIFICATION

### Example 1: Binding of Biparatopic Fusion Proteins

The ability of an expressed biparatopic construct comprising the fusion protein containing a CD19 variant, an anti-CLL-1 scFv, and an anti-CLL-1 VHH (#357) to bind CLL-1 positive U937 cells was evaluated relative to the binding of an expressed construct containing a fusion protein comprised of a CD19 variant and an anti-CLL-1 VHH (#330) and an expressed construct containing a fusion protein comprised of a wild type CD19 and an anti-CLL-1 scFv (#186). Briefly, binding affinity was determined by the following method: U937 cells were washed with FB (FACS buffer PBS+1% BSA+0.1% sodium azide) and then suspended in FB and blocked with human Fc block (Becton Dickinson) at room temperature (RT) for 10 minutes. Approximately 5×10⁵ cells were aliquoted per sample. The cells were spun, washed with FB, and then suspended in 100 μl of the biparatopic or monospecific fusion protein dilution (supernatant or purified starting at 10 μg/ml) in FB. The cell/fusion protein mixture was incubated for 30 minutes at 4° C. and then washed 2× with FB. The cells were suspended in 100 μl FB and stained with FMC63-PE (Millipore 5 μl/test) and incubated 30 minutes at 4° C. After washing 2× with FB, the cells were fixed with a final concentration of 1% PFA in PBS (Thermo Scientific) and then analyzed by flow cytometry (Accuri C6, Becton-Dickinson).

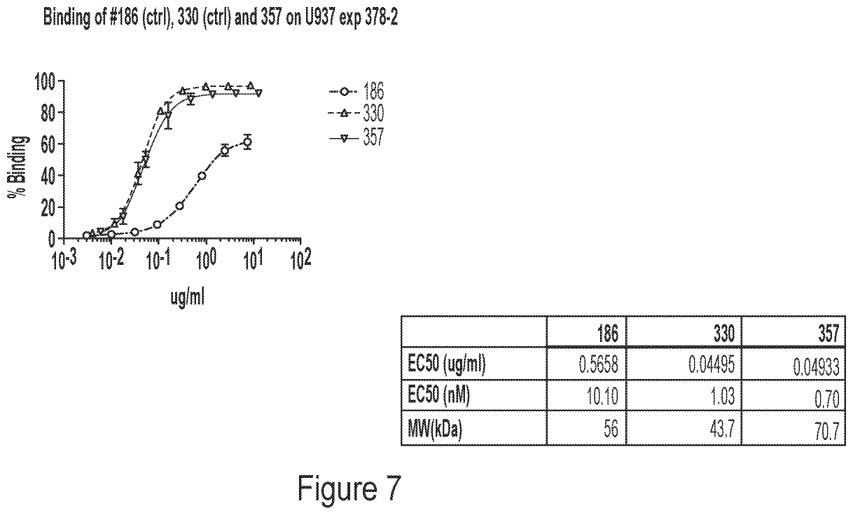

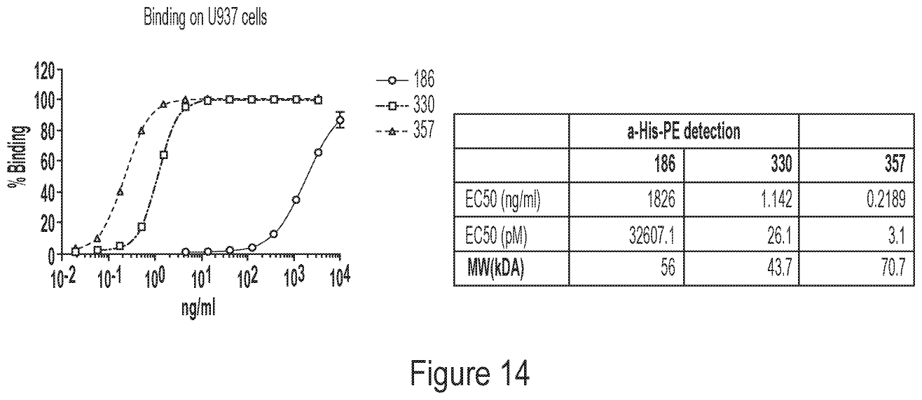

As seen in FIG. 7 the expressed biparatopic fusion protein and the fusion protein comprising an anti-CLL-1 VHH appear to bind with similar affinity (0.7 nM and 1.0 nM respectively). The difference in EC₅₀ values for fusion proteins #330 and #357 do not appear to be statistically significant. Notably, both the expressed biparatopic and VHH containing fusion protein bind with greater affinity than the fusion protein containing wild type CD19 and an anti-CLL-1 scFv which has an affinity of ˜10 nM. Notably, once the fusion proteins are purified and the protein concentration determined to provide dilutions more accurate than supernatant, the biparatopic fusion protein binds better than the fusion protein comprising an anti-CLL-1 VHH, and both bind better than the fusion protein containing wild type CD19 and an anti-CLL-1 scFv. (FIG. 14).

The binding of the biparatopic fusion proteins to CLEC12A was also examined by ELISA. A 96-well ELISA plate was coated with 1 μg/ml of anti-CD19 monoclonal antibody FMC63 in 0.1 M Carbonate, pH9.5. The plate was left to incubate overnight at 4° C. The coated plate was blocked with TBS/0.3% nonfat dry milk (NFD) for 60 minutes at RT. The plate was washed with TBST (0.1 M Tris, 0.5 M NaCl, 0.05% Tween 20. The purified biparatopic fusion protein was diluted in TBS/BSA and added at varying amounts from 0.005 μg/ml to 1 μg/ml, covering more than three logs of final concentration. The biparatopic fusion protein was allowed to incubate for 1 hour at RT, then the plate was washed and the HRP-coupled anti-His antibody was added for 60 minutes at RT, then used for enzymatic detection, following the manufacturer's directions. The apparent EC₅₀ was calculated using the 4-parameter curve fitting function of Softmax software.

The results of this binding experiment are shown in Table 8. The biparatopic fusion protein #357 binds with an EC₅₀ of 0.01 nM as does the single VHH #330 fusion protein. The scFv fusion protein has a much weaker binding affinity of ˜2.5 nM.

TABLE 8

| | CLEC12A | CLEC12A | CLEC12A-cell |
|---|---|---|---|
| | binding (ELISA | binding (FACs | cytotoxicity |
| | EC50) | EC50) | (IC50 n = 2-3) |
| #186 | 2.52 nM | 10 nM | 100 pM |
| #321 | 0.09 nM | 3 nM | 27 pM |
| #330 | 0.013 nM | 1 nM | 7.8 pM |
| #357 | 0.01 nM | 0.7 nM | 0.65 pM |

### Example 2: Cytotoxicity Mediated by Expressed Biparatopic Fusion Proteins

Several fusion proteins were purified and evaluated for their ability to bridge killing of CLL-1 expressing cells by CAR-T cells that target CD19. Luciferase was introduced to U937 cells and 29T-CLL cells by lentiviral transduction. On day 1 U937 cells were seeded at 1×10⁴ per well of a round-bottom 96 well plate (Thermo Fisher) or 293T-CLL-1 cells were seeded in a flat bottom plate in cell culture media (RPMI 1640, 10% FBS). On day 2, the fusion proteins #357, #330 and #321 were added at 0.2 μg/ml (20 ng/well) with serial 3 fold dilutions where indicated, then left to incubate at 37° C. for 1 hour using the cell culture incubator.

CAR-CD19-directed-T cells or untransduced cells (UTD) were freshly thawed from pre-aliquoted vials kept in liquid nitrogen and washed once with medium to remove DMSO. The CAR19 T cells were then added to the 96 well plate where indicated, using a T cell:target cell (aka effector:target) cell ratio of 10:1 or 1:1, where the target cells were U937 cells.

On day 3, the plate was spun at 550 RCF for 5 minutes and rinsed with PBS then spun again to remove the PBS. 20 μl 1× lysis buffer (Luciferase assay system Promega) was added and the lysate was transferred into 96 well opaque tissue culture plates. The 96 well opaque tissue culture plate, containing 20 μl of cell lysate per well, was placed into a luminometer with injector (Glomax Multi Detection System from Promega). The injector added 100 μl of Luciferase assay reagent per well, then the well was read immediately. The plate was then advanced to the next well for a repeat of the inject-then-read process. The 293T-CLL-1 cell line cytotoxicity assay was similar except that the cells were seeded into flat bottom plates and the cells were not spun out prior to lysis.

The % cell death (aka cytotoxicity) was calculated as follows:

% killing=[1−luc reading (experimental wells)/luc reading (tumor cells without CAR T cells)]×100.

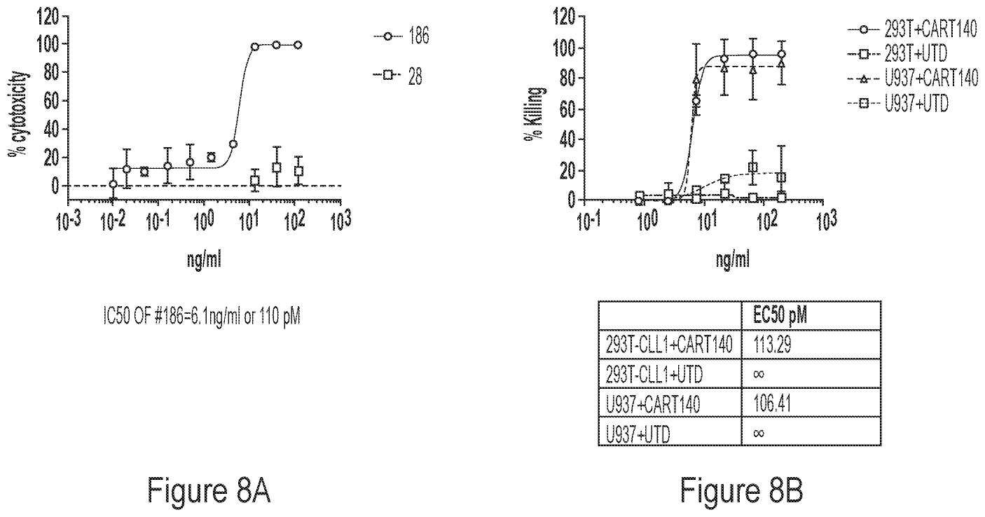

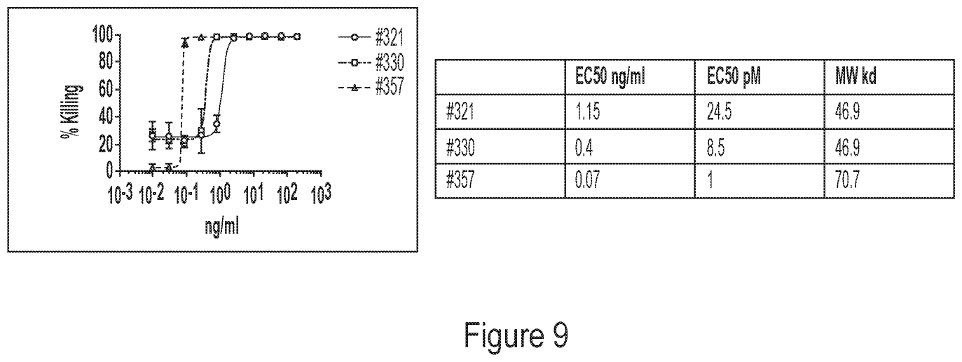

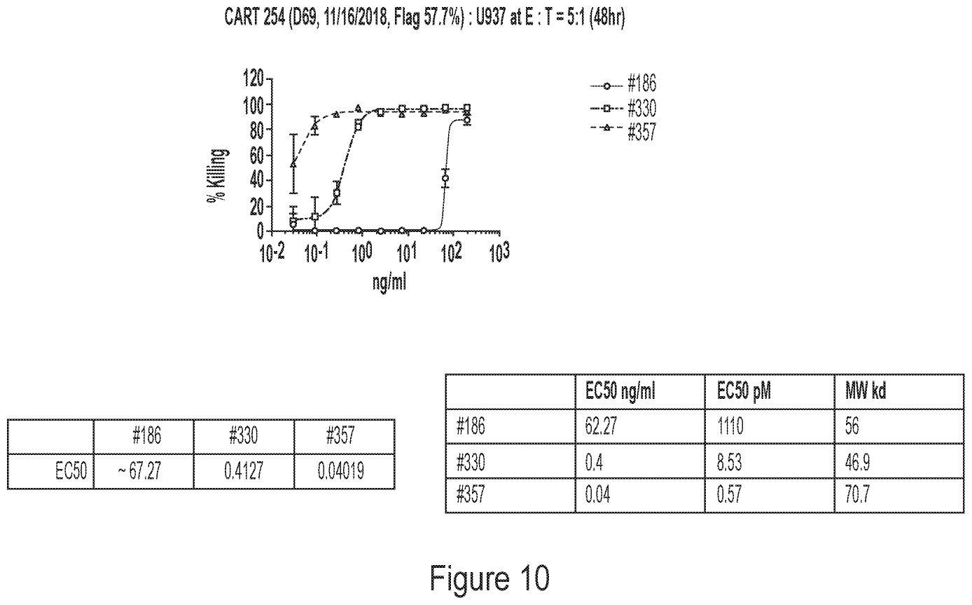

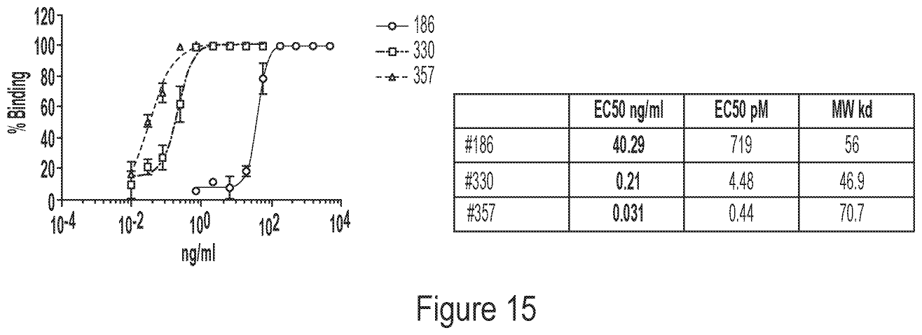

Purified fusion protein #186 consistently produced an IC₅₀ value of approximately 100 pM on U937 and on 293T cells expressing CLL-1, as shown in FIG. 8. In FIG. 8A, the negative control is protein #28 (CD19 protein). In FIG. 8B untransduced T cells (UTD) serve as the negative control. Purified fusion protein generated from biparatopic construct #357 mediated more potent killing than fusion protein #330 (˜8-fold) and fusion protein #321 (˜25-fold), as shown in FIG. 9. In a similar experiment but using a lower E:T ratio of 5:1, the increase in potency of the biparatopic fusion protein was even more pronounced. The results (FIG. 10) showed that biparatopic fusion protein #357 mediated CAR-CD19 cytotoxicity by ˜15-fold greater than fusion protein #330. In this particular experiment, the potency of fusion protein #186 was low at 1 nM. These results are further confirmed by the experimental results shown in FIG. 15.

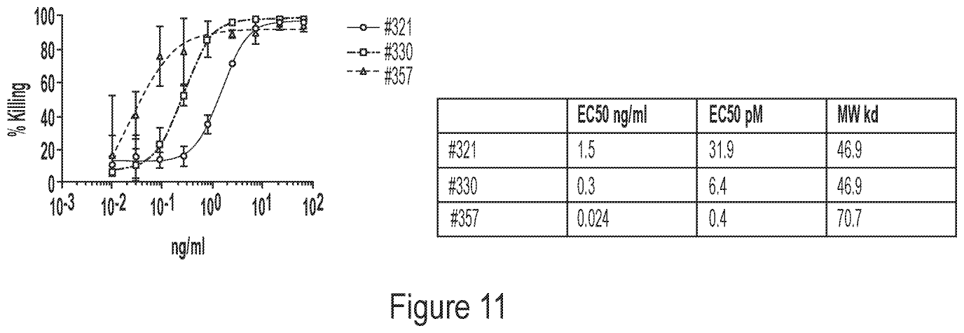

A third cytotoxicity assay performed using the 5:1 E:T ratio showed similar trends in potency (FIG. 11) such that biparatopic fusion protein #357 mediated CAR-CD19 cytotoxicity by ˜15-fold greater than fusion protein #330.

### Example 3: Cytotoxicity of Constitutively Expressed Biparatopic Constructs

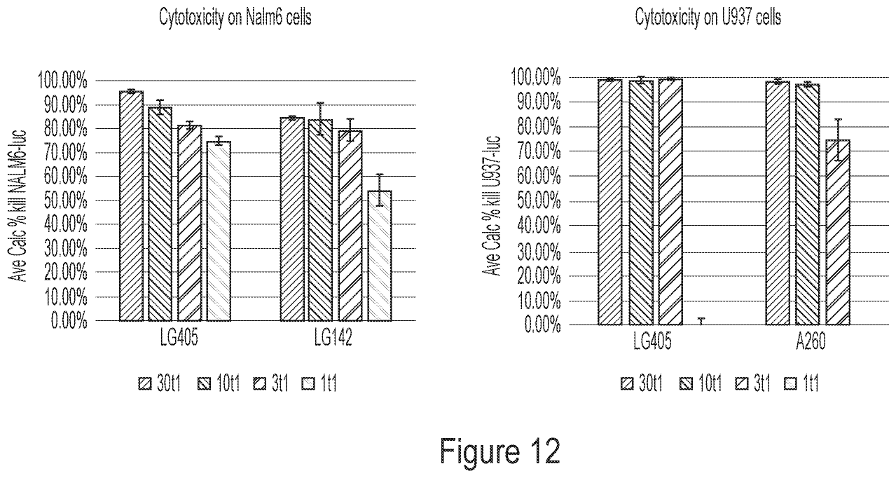

To test the cytotoxicity of constitutively expressed biparatopic constructs sequences encoding a CD19 binding CAR upstream of biparatopic fusion proteins were introduced to T cells by lentiviral transduction. Briefly, the anti-CD19 CAR sequence (CAR-CD19) derived from the FMC63 antibody (VL-VH) with a FLAG-tag, CD28 linker and transmembrane domain plus CD28, 4-1BB and CD3 zeta intracellular domains, followed by a P2A site and then the anti-CLL-1 VHH 2H3-variant CD19 fusion protein sequences, was chemically synthesized and cloned into a lentiviral vector by Lentigen Technologies vector. Viral particles were produced by Lentigen for further studies. For the anti-CLL-1 CAR control (A260), the sequence was synthesized and cloned into a modified vector from Systems Biosciences. To make the CAR only lentiviral particles, supernatants containing lentivirus were generated by transient transfection of HEK 293 FT cells, as described by the SBI protocol. Pelleted lentiviral particles were suspended in PBS and used for primary T cell transductions. Selected CD3+ human primary T cells were cultivated in ImmunoCult-XF T cell expansion medium (serum/xeno-free) supplemented with 20 IU/ml IL-2 at a density of 3×10⁵ cells/ml, activated with CD3/CD28 T cell Activator reagent (STEMCELL Technologies) and transduced on day 1 with CAR-CD19 plus fusion protein or the anti-CLL-1 CAR lentiviral particles, in the presence of 1× Transdux (SBI). Cells were propagated until harvest on day 10, at which time the surface expression of CAR-CD19 was assessed by flow cytometry with anti-FLAG antibody (Invitrogen). The expression construct comprised a “P2A” cleavage site separating the CD19 CAR from the biparatopic fusion proteins so that the resultant transcription/translation would result in the CAR-CD19 expressed on the T cell surface and the secretion of the biparatopic fusion protein. Three constructs were tested: LG405 expresses CAR-CD19 and secretes the variant CD19-bi-paratopic anti-CLEC12A fusion protein, LG142, a control construct, expresses CAR-CD19 and secretes a wild type CD19-anti-Her2 scFv fusion protein; and A260 is a control CAR that directly recognizes CLL-1 (CLEC12A) (CAR-CLEC12A). The cells expressing the LG405 construct were co-cultured with CD19 positive NALM6 cells and CLL-1 positive U937 cells at varying effector to target ratios. The positive control for Nalm6 cytotoxicity is LG142 that also expressed CAR-CD19. The positive control for U937 cytotoxicity is A260, a CAR-CLEC12A. FIG. 12 shows that LG405-transduced T cells that express CAR-CD19 and secretes=the biparatopic fusion protein were able to induce killing of both CD19 positive NALM6 cells and CLL-1 positive U937 cells. Notably, the cytoxic activity achieved against U937 cells was 100% even at a 3:1 E:T ratio.

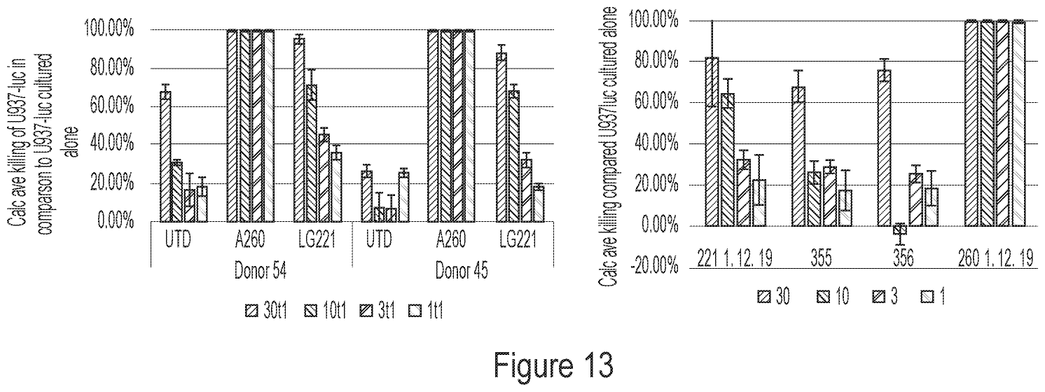

CAR T cells secreting the single scFv-based or single VHH-based fusion proteins are less potent against U937 cells. Three constructs were tested: LG221-transduced T cells expressed CAR-CD19 and secrete a CD19-anti-CLL-1 scFv fusion protein, LG355-transduced T cells express CAR-CD19 and secrete a CD19-anti-CLL-1 VHH (1B12) fusion protein; and LG356-transduced T cells express CAR-CD19 and secrete an anti-CLL-1 VHH (2H3)-CD19 fusion protein. In two assays, CAR T cells made from LG221, LG355, and LG356 lost activity against U937 cells below a 30:1 E:T ratio and are therefore much less potent than CAR T cells made from LG405 that secretes the variant CD19-bi-paratopic anti-CLEC12A fusion protein (FIG. 13).

### Example 4: Binding of Additional Biparatopic Constructs

To further confirm the binding and cytotoxicity capabilities of an expressed biparatopic fusion protein comprising a CD19 variant, an anti-CLL-1 scFv, and an anti-CLL-1 VHH, a construct was generated with the same features as construct #357 but lacking the HIS tag. U937 (ATCC) and OCI-AML-5 (DSMZ) cells were cultured as detailed by the supplier. The cells were washed with PBS, suspended in 50 μl FACS buffer (PBS+1% BSA and 0.1% sodium azide) and blocked with human Fc block (Becton Dickinson) at room temperature for 10 minutes. Then, 50 μl of the fusion protein, #357 or #518, starting at 1 μg/ml with 3 fold dilutions in FACS buffer was added. The cell/fusion protein mixture was incubated for 30 minutes at 4° C. and then washed twice with FACS buffer. The cells were suspended in 100 μl of FACS buffer, stained with FMC63-PE (Millipore 5 l/test), and incubated for 30 minutes at 4° C. The cells were washed twice with FACS buffer, fixed with a final concentration of 1% PFA in PBS (Thermo Scientific) and then analyzed by flow cytometry.

To test cytotoxicity a 96 well round bottom plate was seeded with 50 μl of U937 cells carrying the luciferase gene (U937-luc) at 1×10⁴ cells/well in RPMI 1640 medium/10% FBS without antibiotics (RPMI/FBS). Dilutions of the biparatopic fusion proteins #357 or #518 were made in 25 μl RPMI/FBS, starting at 60 ng/ml with 3 fold dilutions and added to the cells. The CAR-CD19 T cells (CAR254) were thawed and washed once with RPMI/FBS via centrifugation at 550 RCF for 10 minutes. The CAR T cells were added to the wells to give a CAR:target cell ratio (E:T) of 10:1. The plates were incubated at 37° C. for 48 hours. The plate was centrifuged at 550 RCF for 5 minutes, the pellet was rinsed with PBS, and spun again. Then, 20 μl of 1× lysis buffer (Promega Cat. #E1500) was added to the pellet, and the lysate was transferred into a 96 well opaque tissue culture plate (Fisher Scientific Cat. #353296). The plates were read in a luminometer with an injector to dispense the substrate (Promega Cat. #E1500). The percent killing was calculated based upon the average loss of luminescence of the experimental vs the control (untreated) cells.

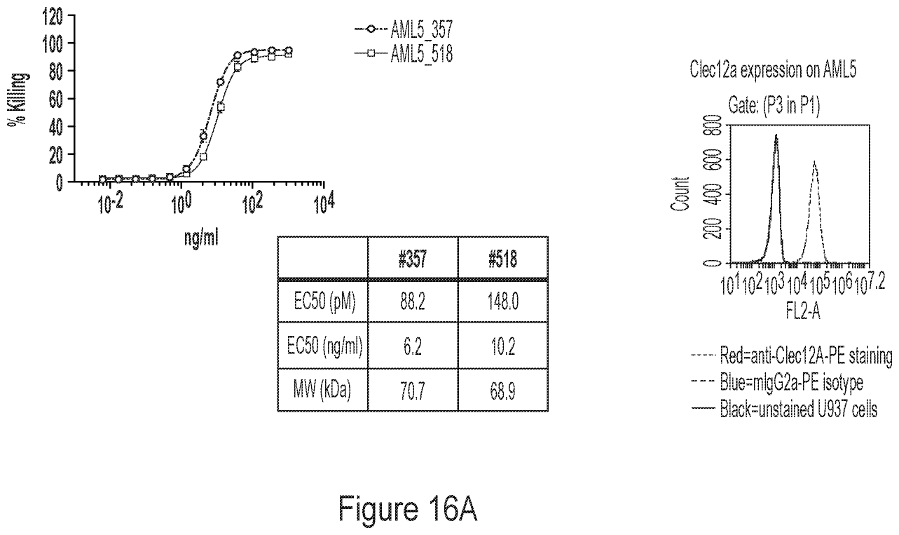

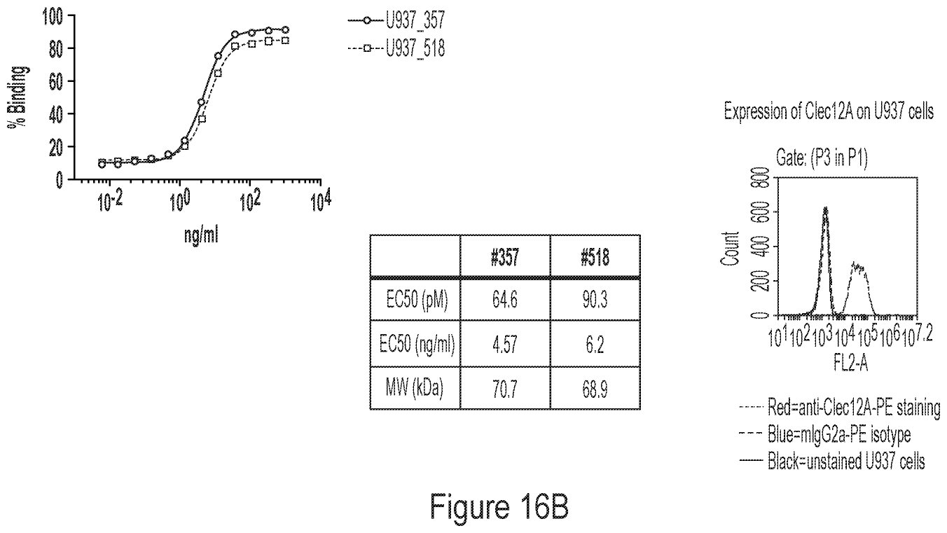

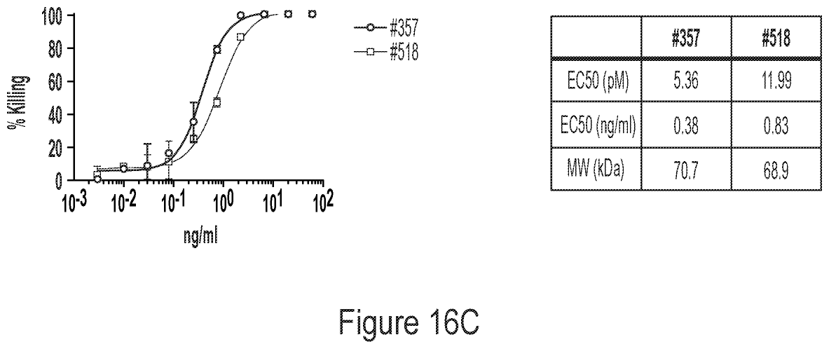

FIG. 16A shows the HIS-tagged biparatopic construct (#357) and the construct with no HIS-tag (#518) demonstrate equivalent binding by FACS on the CLEC12A-positive cell line AML5. A similar equivalence is observed using the CLEC12A-positive cell line U937 (FIG. 16B). The equivalent ability of both fusion proteins to bridge CAR-CD19 T cells to U937 target cells and mediate potent cytotoxicity is demonstrated in FIG. 16C.

### Example 5: Cytotoxicity of Constitutively Expressed Biparatopic Constructs without his-Tag

To further confirm the cytotoxicity of constitutively expressed biparatopic constructs, sequences encoding a CD19 binding CAR upstream of biparatopic fusion proteins lacking a His tag were introduced to T cells by lentiviral transduction. In addition, the FLAG-tag was removed from the CAR-CD19 sequence. The resulting construct was termed #468.

To produce CAR-CD19 T cells that secrete CD19-anti-CLL fusion proteins, CD3-positive human primary T cells from 2 donors were cultivated in ImmunoCult-XF T cell expansion medium (serum/xeno-free) supplemented with 20 IU/ml IL-2 at a density of 3×10⁵ cells/ml, activated with anti-CD3/anti-CD28 T cell Activator reagent (STEMCELL Technologies) and transduced on day 1 with CAR-405 lentiviral particles (Lentigen, 1.6-3.3×10⁶ TU/ml) or CAR-468 lentiviral particles (Flash, qPCR is: 4.3×10⁹ TU/ml) in the presence of 1× Transdux (SBI). Cells were propagated until harvest on day 10. For control CARs, CAR-254, CAR-260 and CAR408, particles were added based on a SupT1 titer. The percent CAR-CD19 expression was measured by staining the CAR T cells with anti-FLAG antibody or with CD19-Fc (CAR-468). Briefly, 500,000 cells were incubated with anti-FLAG antibody diluted 1:100 (Thermo Fisher) followed by anti-rabbit APC (1:100 dilution, Thermo Fisher) or 0.25 μg/ml CD19-Fc (R&D Systems) followed by 1:200 dilution of anti-Fc gamma (Jackson ImmunoResearch). Cells were fixed with 2% paraformaldehyde and the percent FLAG-positive cell populations was measured using a BD Accuri C6 flow cytometer. In this comparison, the lowest % of all the CAR-T preparations was 48%. As demonstrated in Table 9, the other CAR-Ts were normalized to 48% positive CAR expression by dilution with UTD cells, prior to the adding the CAR T cells to the assays.

To asses cytotoxicity, U937 (ATCC), PL-21 and OCI-AML-5 (DSMZ) cells were cultured as detailed by the supplier. Luciferase expressing lines were generated using a lentivirus (GeneCopoeia) and puromycin selection. Cells carrying the luciferase gene were seeded at 1×10⁴ cells in 50 μl per well in a 96 well round bottom plate in RPMI 1640+10% FBS without antibiotics. CAR T cells were thawed and washed once with RPMI/FBS via centrifugation at 450 RCF for 10 minutes. The CAR T cells were added to give an E:T cell ratio of 30:1, 10:1, 3:1, or 1:1 respectively. The plates were incubated at 37° C. for 48 hours. The plate was centrifuged at 450 RCF for 5 minutes, and the pellet was rinsed with PBS and spun again. Then, 20 μl of 1× lysis buffer (Promega) was added to the pellet, and the lysate was transferred into a 96 well opaque tissue culture plate (Fisher). The plates were read in a luminometer with an injector (Glomax Multi Detection System, Promega). The percent killing was calculated based upon the average loss of luminescence of the experimental vs the control (target cell only).

Biparatopic fusion protein concentration was determined in the CAR-405 expansion culture medium (batch fusion protein) as well as through stimulation post-freezing (fusion protein secretion). For measurement of biparatopic fusion protein during expansion at the time of harvest (day 10), 0.5 ml of cell-free culture media was collected and frozen at −20° C. For biparatopic fusion protein secretion after activation, CAR-405 T cells were thawed and resuspended at 3×10⁶ cells/ml in RPMI-1640/10% FBS. About 6×10⁵ cells (200 μl of the resuspension) were plated in 96 well u-bottom plates and stimulated with CD3/CD28 T cell Activator reagent (STEMCELL Technologies). After 4 days, supernatants were harvested and the amount of biparatopic protein was measured by ELISA. A 96 well plate was coated with 1.0 μg/ml anti-CD19 FMC63 (Novus) in 0.1 M carbonate, pH 9.5 overnight at 4° C. The plate was blocked with 0.3% non-fat milk in TBS for 1 hour at RT. After washing in TBST (0.1 M Tris, 0.5 M NaCl, 0.05% Tween20) 3 times, 100 μl cell culture supernatant was added to the plate and incubated for 1 hour at RT. Purified CD19-anti-Her2 scFv protein was used to generate the standard curve. The plate was washed 3 times in TBST then HRP-anti-His (BioLegend) was added at 1:2000 at RT in the dark for 1 hour. To enzymatically quantify the peroxidase bound, 1-Step Ultra TMB-ELISA (Thermo Fisher) solution, was added and the plate read at 405 nm. Curves were fit using a four-parameter logistic (4 PL) regression to calculate the EC₅₀.

Because the biparatopic fusion protein secreted by CAR-468 T cells lacks the His-tag on the C-terminus, an additional ELISA assay was developed. Samples were collected as described above for CAR-405 T cells. A 96 well plate was coated with 1.0 μg/ml purified CLEC12A (Sino Biological) in 0.1 M carbonate, pH 9.5 overnight at 4° C. The plate was blocked with 0.3% non-fat milk in TBS for 1 hour at RT. After washing in TBST (0.1 M Tris, 0.5 M NaCl, 0.05% Tween20) 3 times, 100 μl cell culture supernatant was added to the plate and incubated for 1 hour at RT. Purified fusion protein #357 was used to generate a standard curve. The plate was washed 3 times in TBST and then 1 μg/ml anti-CD19 antibody FMC63 (Novus) was added at RT in the dark for 1 hour. The plate was washed again and RP-anti-mouse IgG antibody (Jackson ImmunoResearch) was added for 30 minutes at RT. To enzymatically quantify the peroxidase bound to the wells, 1-Step Ultra TMB-ELISA (Thermo Fisher) solution, was added and the plate read at 405 nm. Curves were fit using a four-parameter logistic (4 PL) regression to calculate the EC50.

To determine IFNγ production CAR T cells were added to U937 cells to give an E:T cell ratio of 3:1 respectively. The plates were incubated at 37° C. for 48 hours. The plate was centrifuged at 450 RCF for 5 minutes and the supernatant was collected. A cytometric bead assay (CBA) (Becton Dickinson, Catalog No. 551809) was then conducted on BD Accuri C6 instrument to measure cytokines present in the co-culture supernatant. FCAP Array software (Soft Flow Hungary Ltd, Catalog No. 641488) was then used to generate standard curves and to determine the concentration of unknown samples.

T cells were transduced with the lentiviral particles that express the CAR-CD19 and secrete the HIS-tagless form of the biparatopic construct (#518). The CAR T cells (CAR468) were made using primary T cells from different donors, assessed for expression and activity and compared to CAR405 T cells from the same donors. The CAR T cells (CAR468 and CAR405) were equivalent across a variety of parameters, as summarized in Table 9.

TABLE 9

| | | | fusion | 48% | IFNγ | fusion protein |
|---|---|---|---|---|---|---|
| | | | protein | CarT | (pg/ml) | secretion after |
| | | | (ng/ml/e6 | Killing | in 48% | activation |
| | | | Car19+) | of | CarT | (48% CAR- |
| | | Batch | during | U937-luc | 3:1 U937 | positive, |
| Donor | CAR | % Car | expansion | at 3:1 | co-culture | on day 4) |
| 45 | CAR468 | 48.8 | 13.1 | 97.8 | 7.2 | 2.3 |
| 38 | CAR468 | 47.7 | 14.4 | 77.3 | 11.6 | 14.5 |
| 45 | CAR405 | 60 | 17.4 | 100 | 38.6 | 0.3 |
| 38 | CAR405 | 54 | 12.7 | 99.5 | 107.7 | 53.5 |

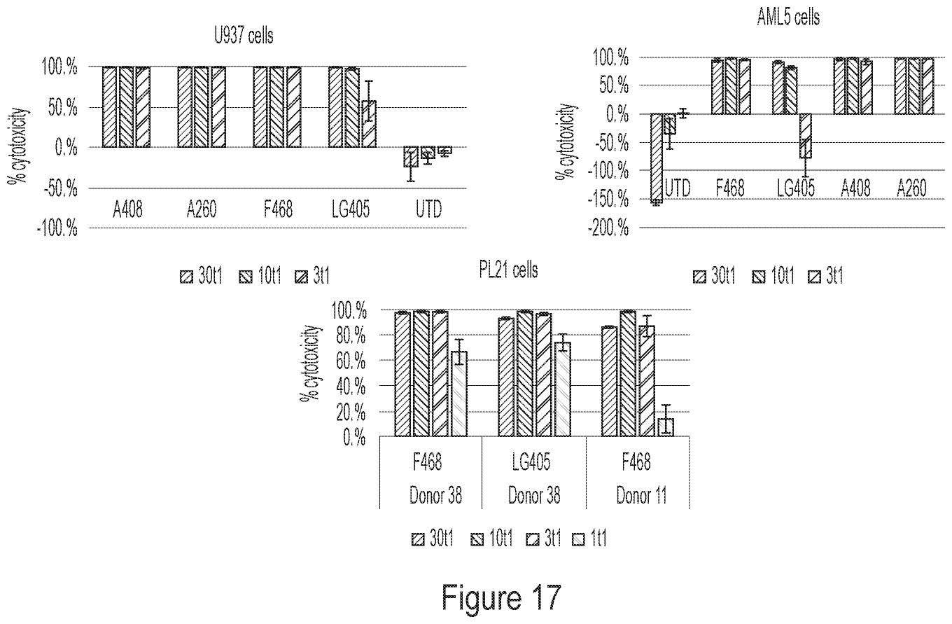

Additionally, as seen in FIG. 17, CAR468 T cells, expressing the biparatopic fusion protein with no HIS tag, kills multiple CLEC12A-expressing AML cell lines (U937; PL21; AML5) as well as or better than CAR405, expressing the same biparatopic fusion protein with a HIS tag.

### Example 6: In Vivo Cytotoxicity of Biparatopic Constructs

To confirm the in vivo efficacy of CAR-CD19 T cells that secrete biparatopic fusion proteins were introduced into NSG™ mice. All animal studies were performed in accordance with Tufts University IACUC approved guidelines. 6-8 week old NSG™ mice (Jackson Laboratories, NOD.Cg-Prkdc^{scid} Il2rg^{tm1Wjl}/SzJ) were used. Mice were inoculated with 25,000 U937-luciferase cells IV. On day 3, 10⁷ CAR-405, CAR-468 or untransduced T cells (UTD) from donors 38 or 45, were injected IV into the mice; a cohort of mice had no T cell injection (NA). For imaging, animals received luciferin (150 mg/kg IP) and were anesthetized with isoflourane. A Perkin Elmer IVIS 200 was used for luminescence level determination. The mice were sacrificed when they reached a set luciferase limit. For the U937 cells, this limit was 1×10⁹ luciferase units or total flux. The study was terminated after 28 days.

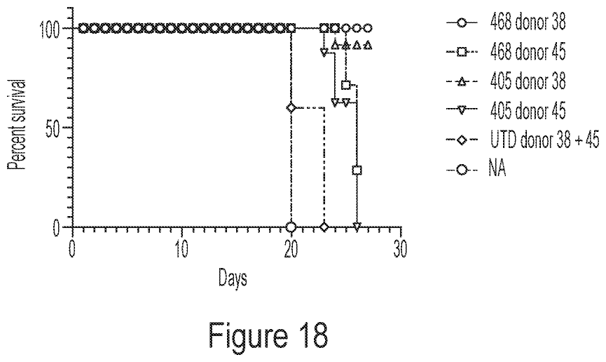

CAR-T cells from two different donors expressing biparatopic constructs with and without HIS-tags demonstrated efficient killing of U937 xenografts. (FIG. 18)

### Example 7: Biparatopic Constructs Bind Multiple CLEC12A Alleles

We previously observed that there are two forms of CLEC12A with a single amino acid difference (see PCT/US2019/063691). The canonical sequence (UniProt; Q5QGZ9-1) contains a Lysine (K in bold underline; SEQ ID NO:328).

(SEQ ID NO: 328)
MWIDFFTYSSMSEEVTYADLQFQNSSEMEKIPEIGKFGEKAPPA
PSHVWRPAALFLTLLCLLLLIGLGVLASMFHVTLKIEMKKMNKL
QNISEELQRNISLQLMSNMNISNKIRNLSTTLQTIATKLCRELY
SKEQEHKCKPCPRRWIWHKDSCYFLSDDVQTWQESKMACAAQNA
SLLKINNKNALEFIKSQSRSYDYWLGLSPEEDSTRGMRVDNIIN
SSAWVIRNAPDLNNMYCGYINRLYVQYYHCTYKKRMICEKMANP
VQLGSTYFREA

However, the sequence of a commercially available recombinant CLEC12A protein from Sino Biological (GenBank: EAW96132.1) has a Glutamine instead (Q in in bold underline; SEQ ID NO:329).

(SEQ ID NO: 329)
MWIDFFTYSSMSEEVTYADLQFQNSSEMEKIPEIGKFGEKAPPA
PSHVWRPAALFLTLLCLLLLIGLGVLASMFHVTLKIEMKKMNKL
QNISEELQRNISLQLMSNMNISNKIRNLSTTLQTIATKLCRELY
SKEQEHKCKPCPRRWIWHKDSCYFLSDDVQTWQESKMACAAQNA
SLLKINNKNALEFIKSQSRSYDYWLGLSPEEDSTRGMRVDNIIN
SSAWVIRNAPDLNNMYCGYINRLYVQYYHCTYKQRMICEKMANP
VQLGSTYFREA

In previous studies we found that VHH clone 2H3 did not bind a CLEC12A from AbClonal (NCBI Reference Sequence: NP_001193939.1, isoform3) or a cDNA from Genscript both of which contain a Lysine residue at amino acid 254. This suggests that the K/Q amino-acid is within or close to the 2H3 epitope and is consistent with the 2H3 epitope being close to the C-terminus. In contrast, we found that the scFv recognizing CLEC12A binds to both protein variants.

Given the unique binding properties of VHH clone 2H3 relative to the anti-CLEC12A scFv we tested the binding properties of a biparatopic fusion protein containing both the VHH clone 2H3 and an anti-CLEC12A scFv. HEK-293T cells were transfected with full-length CLEC12A (UniProt Q5QGZ9-1) cDNAs expressing the amino acid 254 “K” version of CLEC12A (GenScript) or a version with “Q” at this position generated by mutagenesis. The cells were removed with Accutase and washed with PBS, suspended in 50 μL FACS buffer (FB, PBS+1% BSA and 0.1% sodium azide) and blocked with human Fc block (Becton Dickinson) at room temperature for 10 minutes. Then, 50 μl of the purified fusion proteins #186, #330 or #357 diluted in FACS buffer (starting at 3 μg/ml with 3 fold dilutions) were added. The cell/fusion protein mixture was incubated for 30 minutes at 4° C. and then washed twice with FACS buffer. The cells were suspended in 100 μl of FACS buffer, stained with HIB19-PE (BioLegend 5 l/test), and incubated for 30 minutes at 4° C. The cells were washed twice with FACS buffer, fixed with a final concentration of 1% PFA in PBS (Thermo Scientific) and then analyzed by flow cytometry.

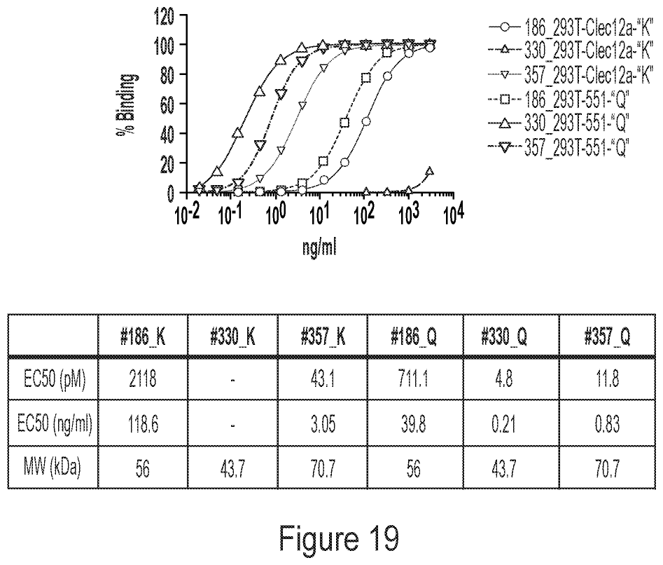

FIG. 19 shows that the anti-CLEC12A VHH (in fusion protein #330) binds very well to the Q form, but very weakly to the K form (greater than 10,000-fold weaker). In contrast the anti-CLEC12A scFv (in fusion protein #186) binds weakly, but with similar potency, to both forms. Notably, the biparatopic fusion protein (#357) binds very well to both forms (red and yellow lines). The biparatopic construct binds better to the Q form (11.8 pM) but still binds very well to the K form (43.1 pM). These data confirm that a biparatopic fusion protein can enhance binding to the target protein (CLEC12A).

### Example 8: Additional Examples of Dual Antigen Binding Constructs

To further demonstrate the utility and effectiveness of constructs that bind more than one antigen a fusion protein was produced comprising an anti-CLEC12A VHH; an anti-CD33 scFv; and a CD19 ECD polypeptide. The fusion protein containing the CD19 ECD and anti-CD33 scFv was termed #410. The fusion protein comprising an anti-CLEC12A VHH, an anti-CD33 scFv, and a CD19 ECD was termed #440.

ELISAs for binding to CD19, CLEC12A and CD33 were run using purified fusion proteins #330, #410 and #440. A 96 well plate was coated with 1.0 μg/ml FMC63 (Novus) in 0.1 M carbonate, pH 9.5 overnight at 4° C. The plate was blocked with 0.3% non-fat milk in TBS for 1 hour at room temperature. After washing in TBST (0.1 M Tris, 0.5 M NaCl, 0.05% Tween20) 3 times, the fusion protein supernatant was titrated using 3-fold dilutions in 1% BSA in TBS and incubated 1 hour at room temperature. For detection by anti-His, after washing 3 times in TBST, HRP-anti-His (BioLegend) was added at 1:2000. The plate was incubated at room temperature in the dark for 1 hour. For detection of binding to CLEC12A, biotinylated CLEC12A was added at 0.5 μg/ml and incubated for 1 hour. This was followed by adding HRP-SA (Pierce) at 1:2000 and incubated at room temperature in the dark for 1 hour. For testing binding to CD33, biotinylated CD33 was added at 0.5 μg/ml and incubated for 1 hour. This was followed by adding HRP-SA (Pierce) at 1:2000 and incubated at room temperature in the dark for 1 hour. Then, 1-Step Ultra TMB-ELISA (Thermo Fisher) solution, was added and the plate read at 405 nm. Curves were fit using a four-parameter logistic (4 PL) regression to calculate the EC₅₀.

To test binding of the fusion protein to cells, U937 or Molm14 cells were washed with PBS, suspended in 50 μL FACS buffer (FB, PBS+1% BSA and 0.1% sodium azide) and blocked with human Fc block (Becton Dickinson) at room temperature for 10 minutes. Then, 50 μl of the purified fusion protein #330, #410 or #440 dilution (starting at 3 μg/ml with 3-fold dilutions) in FB was added. The cell/fusion protein mixture was incubated for 30 minutes at 4° C. and then washed twice with FB. The cells were suspended in 100 μl of FB, stained with the anti-CD19 antibody FMC63-PE (Millipore 5 μl/test), and incubated for 30 minutes at 4° C. The cells were washed twice with FB, fixed with a final concentration of 1% PFA in PBS (Thermo Scientific) and then analyzed by flow cytometry.

To test cytoxicity, luciferase labeled Molm14 or U937 cells were targeted for cytotoxic killing using a CAR19 T cell preparation and the purified proteins #330, #410 and #440 added in a dose titration. 1×10⁴ target cells were added to each well (96 clear round bottom plate) in RPMI medium. The protein titration was started at 180 ng/ml with 3-fold serial titration into corresponding wells (25 μl per well). CAR-CD19 T cells were added at various E:T ratios to the target cells. After 48 hours the plate was centrifuged (450 RCF for 5 minutes) and the cells were washed 1× with PBS then centrifuged a second time. 20 μl lysis buffer was added into each well. The cell lysate was transferred to a 96 opaque white plate. The plates were read in a luminometer with an injector (Glomax Multi Detection System, Promega). The percent killing was calculated based upon the average loss of luminescence of the experimental vs the control (target cell only or target cell plus fusion protein only).

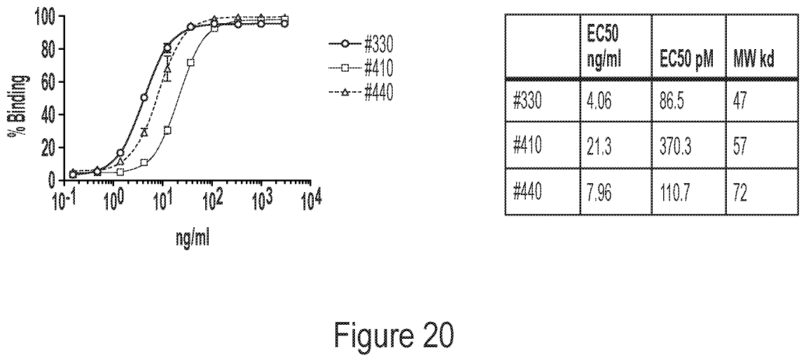

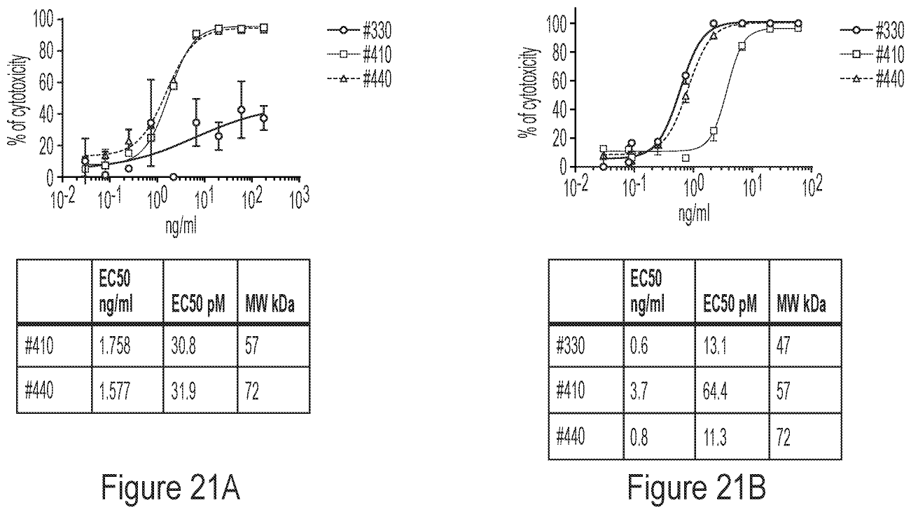

Table 10 provides ELISA results demonstrating the binding capacity of the dual antigen binding fusion protein (#440); the anti-CLEC12A VHH 2H3 fusion protein (#330); and the anti-CD33 scFv fusion protein (#410) to CLEC12A and CD33. The dual antigen binding fusion protein binds equally well to both components (CLEC12A and CD33) with similar EC₅₀ values showing that each individual component in the dual antigen construct is acting independently. FIG. 20 demonstrates that the dual antigen binding fusion protein is capable of binding to U937 cells expressing each individual antigen. FIG. 21 shows the cytotoxicity of the three fusion proteins on U937 (FIG. 21B) and Molm14 (FIG. 21A) cell lines. The cytotoxicity of the fusion proteins on the U937 cells matches the binding data, i.e. the highest cytotoxicity matches the binding of the most potent binder, the VHH, although the dual antigen protein has essentially the same activity. Interestingly, on the Molm14 cell line the scFv to CD33 and the dual antigen binder are about equipotent, while the VHH to CLEC12A is less potent (VHH 2H3 only). This may result from expression of the K variant of CLEC12A by both alleles in the Molm14 cells.

TABLE 10

| | CLEC12A | | CD33 | | |
|---|---|---|---|---|---|
| | binding | EC50 | binding | EC50 | |
| | ng/ml | pM | ng/ml | pM | MW |
| #410 | ∞ | ∞ | 3.0 | 52.2 | 57.5 |
| #440 | 2.4 | 33.4 | 2.1 | 29.2 | 71.9 |
| #330 | 1.4 | 29.9 | ∞ | ∞ | 46.9 |

SEQ ID NO: 1
CCCGAGGAACCTCTAGTGGTGAAGGTGGAAGAGGGAGATAACGCTGTGCTGCAGTG
CCTCAAGGGGACCTCAGATGGCCCCACTCAGCAGCTGACCTGGTCTCGGGAGTCCCC
GCTTAAACCCTTCTTAAAACTCAGCCTGGGGCTGCCAGGCCTGGGAATCCACATGAG
GCCCCTGGCCATCTGGCTTTTCATCTTCAACGTCTCTCAACAGATGGGGGGCTTCTAC
CTGTGCCAGCCGGGGCCCCCCTCTGAGAAGGCCTGGCAGCCTGGCTGGACAGTCAA
TGTGGAGGGCAGCGGGGAGCTGTTCCGGTGGAATGTTTCGGACCTAGGTGGCCTGG
GCTGTGGCCTGAAGAACAGGTCCTCAGAGGGCCCCAGCTCCCCTTCCGGGAAGCTC
ATGAGCCCCAAGCTGTATGTGTGGGCCAAAGACCGCCCTGAGATCTGGGAGGGAGA
GCCTCCGTGTCTCCCACCGAGGGACAGCCTGAACCAGAGCCTCAGCCAGGACCTCA
CCATGGCCCCTGGCTCCACACTCTGGCTGTCCTGTGGGGTACCCCCTGACTCTGTGTC
CAGGGGCCCCCTCTCCTGGACCCATGTGCACCCCAAGGGGCCTAAGTCATTGCTGAG
CCTAGAGCTGAAGGACGATCGCCCGGCCAGAGATATGTGGGTAATGGAGACGGGTC
TGTTGTTGCCCCGGGCCACAGCTCAAGACGCTGGAAAGTATTATTGTCACCGTGGCA
ACCTGACCATGTCATTCCACCTGGAGATCACTGCTCGGCCAGTACTATGGCACTGGC
TGCTGAGGACTGGTGGCTGGAAG
SEQ ID NO: 2
PEEPLVVKVEEGDNAVLQCLKGTSDGPTQQLTWSRESPLKPFLKLSLGLPGLGIHMRPL
AIWLFIFNVSQQMGGFYLCQPGPPSEKAWQPGWTVNVEGSGELFRWNVSDLGGLGCGL
KNRSSEGPSSPSGKLMSPKLYVWAKDRPEIWEGEPPCLPPRDSLNQSLSQDLTMAPGSTL
WLSCGVPPDSVSRGPLSWTHVHPKGPKSLLSLELKDDRPARDMWVMETGLLLPRATAQ
DAGKYYCHRGNLTMSFHLEITARPVLWHWLLRTGGWK
VHH Clone Sequences
SEQ ID NO: 203 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS))
QVQLQESGGGLVQAGGSLRLSCAASGSIFAINEINLMGWYRQAPGKQRELVAACASDG
NTYYADSVKGRFTISRDNAEKTVYLQMNNLKPDDTAVYYCDANSRGNYYSGQGTQVT

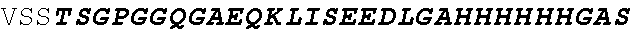

SEQ ID NO: 204 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS))
QVQLQESGGGLVQAGGSLRLSCAASGSIFAINEINLMGWYRQAPGKQRELVAACASDG
NTYYADSVKGRFTISRDNAEKTVYLQMNNLKPDDTAVYYCDANSRGNYYSGQGTLVT

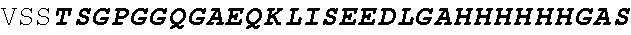

SEQ ID NO: 205 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS))
QVQLQQSGGGLVQAGGSLRLSCAASGSIFAINEINLMGWYRQAPGKORELVAACASDG
NTYYADSVKGRFTISRDNAEKTVYLQMNNLKPDDTAVYYCDANSRGNYYSGQGTQVT

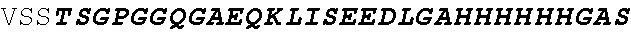

SEQ ID NO: 206 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQQSGGGLVQAGGSLRLSCAASGSIFAINEINLMGWYRQAPGKORELVAACASDG
NTYYADSVKGRFTISRDNAEKTVYLQMNNLKPDDTAVYYCDANSRGNYYSGQGTLVT

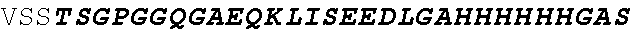

SEQ ID NO: 207 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQQFGGGLVQAGGSLRLSCAASGSIFAINEINLMGWYRQAPGKQRELVAACASDG
NTYYADSVKGRFTISRDNAEKTVYLQMNNLKPDDTAVYYCDANSRGNYYSGQGTLVT

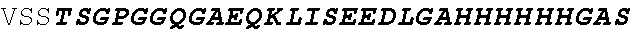

SEQ ID NO: 208 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQASGGGLVQAGGSLRLSCAASGSIFAINEINLMGWYROAPGKORELVAACASDG
NTYYDSVKGRFTISRDNAEKTVYLQMNNLKPDDTAVYYCDANSRGNYYSGOGTQVT

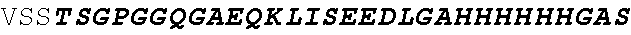

SEQ ID NO: 209 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQEFGGGLVQAGGSLRLSCAASGSIFAINEINLMGWYRQAPGKQRELVAACASDG
NTYYADSVKGRFTISRDNAEKTVYLQMNNLKPDDTAVYYCDANSRGNYYSGQGTLVT

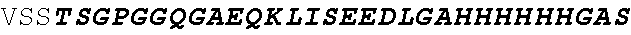

SEQ ID NO: 210 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQEFGGGLVQAGGSLRLSCAASGSIFAINEINLMGWYRQAPGKQRELVAACASDG
NTYYADSVKGRFTISRDNAEKTVYLQMNNLKPDDTAVYYCDANSRGNYYSGQGTQVT

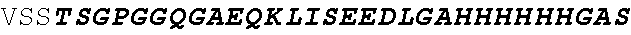

SEQ ID NO: 211 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLVESGGGLVQAGGSLRLSCAASGSIFAINEINLMGWYRQAPGKQRELVAACASDG
NTYYADSVKGRFTISRDNAEKTVYLQMNNLKPDDTAVYYCDANSRGNYYSGQGTLVT

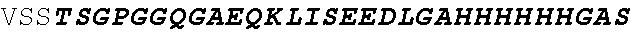

SEQ ID NO: 212 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQQSGGGLAQTGGSLILSCAASGSIFAINEINLMGWYRQAPGKQRELVAACASDGN
TYYADSVKGRFTISRDNAEKTVYLQMNNLKPDDTAVYYCDANSRGNYYSGQGTQVTV

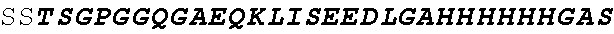

SEQ ID NO: 213 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQESGGGLVQPGGSLRLSCAASGSIFAINEINLMGWYRQAPGKQRELVAACASDG
NTYYADSVKGRFTISRDNAEKTVYLQMNNLKPDDTAVYYCDANSRGNYYSGQGTLVT

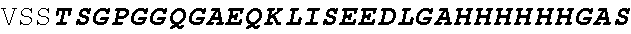

SEQ ID NO: 214 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQESGGGLVQVGESLRLSCVVSGDTRSINLMGWYRQAPGKQRELVAACASDGNT
YYADSVKGRFTISRDNAEKTVYLQMNNLKPDDTAVYYCDANSRGNYYSGQGTLVTVS

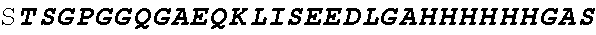

SEQ ID NO: 215 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQESGGGLVQAGGSLRLSCVASGSIRSINVMGWYRQAPGKQRELVAACASDGNTY
YADSVKGRFTISRDNAEKTVYLQMNNLKPDDTAVYYCDANSRGNYYSGQGTQVTVSS

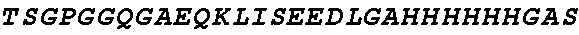

SEQ ID NO: 216 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQESGGGLVQPGGSLRLSCAASGFTFNSYAMTWVRQAPGKGLEWVSDINSGGGST
NYADSVKGRFTISRDNAKNTLYLQMNSLKPEDTAVYYCATELRGSDYYRGPIREYAYW

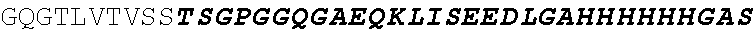

SEQ ID NO: 217 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQESGGALVQAGGSLRLSCAASGLTFSNYAMGWFRQAPGKEREFVAAINWSGGT
TDYATSVKGRFTISRDNAKNTVYLQLNSLKPEDTAVYYCAASYRLRITVVVTPDEYHY

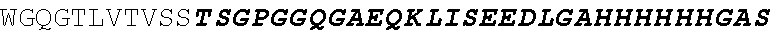

SEQ ID NO: 218 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQQSGGGLVQPGGSLRLSCAASGFAFDDYAMIWVRQGPGKGLEWVSSISWNGGG
TYYAESIVGRFTVSRDNAKKMVYLQMNGLKSEDTAMYYCVKLVDSGWYSAYDYWGQ

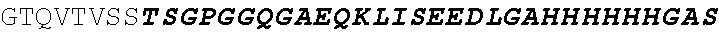

SEQ ID NO: 219 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQESGGGLVQAGGSLRLSCVVSGATSNVNAMGWYRQAPGKERELVAAISSGGSTS
YRDSVKGRFTISRDNAKNTLYLQMNSLKPEDTAMYYCAAQDWATEGYEYDYWGQGT

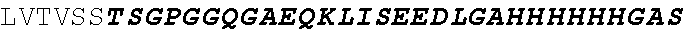

SEQ ID NO: 220 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQAFGGGLVQPGGSLRLSCVVSGTMFSGKDVNWLRQAPGKHVEVVATVSSDGGT
DYADFVKGRFTISRDDAKNTVNLOMNSLEPEDTANYMCHFLWGRHYWGQGTQVTVSS

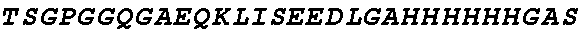

SEQ ID NO: 221 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQAFGGGMVQAGESLRLSCVASGNDISGSAMAWYRAHLGAERELVAVDAPRERP
FYIDPVIGRFTISRDDRNKMLYLQMNDLRPDDTATYWCGPSLRTFHGREWYRPPWFTS

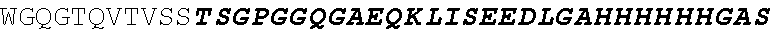

SEQ ID NO: 222 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQQSGGGLVQAGGSLRLSCAASGSIFSINAMGWYRQAPGKRREMVAVVSRFGETT
YTGSVKGRFTISRINRNNTVFLQMNRLKPEDTAVYYCNARIRGNYGSRIDYWGQGTQV

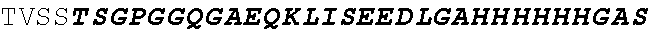

SEQ ID NO: 223 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
QVQLQEFGGGLVQLGGSARLSCVVSGNMLDLNTMAWYRQGELVAALGISTYYAESVK

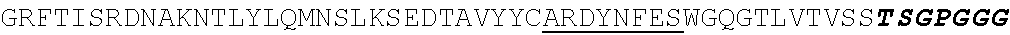

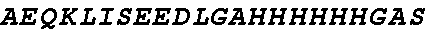

SEQ ID NO: 224 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GAS)):
AVQLQESGGGLVQAGGSLRLSCAASGSDRSINVMNWYROAPGKORELVAAITSGGTTN
YAQSVKGRVTISRDSAKNTVYLQMNSLKPEDTAVYFCKADTRWGGMYWGPGTQVTV

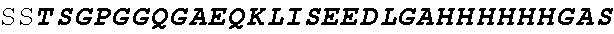

SEQ ID NO: 225 (underlining denotes CDR1, CDR2, CDR3, sequentially; bolded italics at C-
terminus denotes (i) a linker of 9 amino acids (TSGPGGQGA), (ii) a myc-tag
(EQKLISEEDL), (iii) a linker of 2 amino acids (GA), (iv) a hexa-histidine tag (HHHHHH),
and (v) an additional 3 amino acids (GASf).
QVQLQQSGGGLVQAGGSLTLSCAATGRTIDNGAMAWFRQAPGKQRELVAAINWSGGA
TFYTDSVKYRFTISRDNVRHTLDLQMTSLKPEDTTIYFCASRRGVDLRRNSYEYDYWGR

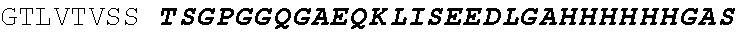

Biparatopic Construct Sequences
Key for Sequence features: signal sequences are underlined; CD19 sequences are italicized,
antibody or fragment thereof (e.g., scFv or VHH) are bolded with CDRs bold and underlined;
spacer/tag sequences (myc-His or His) are bold and italicized, CAR sequences are shaded gray
with flag tag within CAR bolded; Furin cleavage and P2A sites underlined italicized and
bolded
Construct #186
Nucleotide:
SEQ ID NO: 300
ATGCCACCTCCTCGCCTCCTCTTCTTCCTCCTCTTCCTCACCCCCATGGAAGTCAGGCCCGAGGAAC
CTCTAGTGGTGAAGGTGGAAGAGGGAGATAACGCTGTGCTGCAGTGCCTCAAGGGGACCTCAGATG
GCCCCACTCAGCAGCTGACCTGGTCTCGGGAGTCCCCGCTTAAACCCTTCTTAAAACTCAGCCTGG
GGCTGCCAGGCCTGGGAATCCACATGAGGCCCCTGGCCATCTGGCTTTTCATCTTCAACGTCTCTCA
ACAGATGGGGGGCTTCTACCTGTGCCAGCCGGGGCCCCCCTCTGAGAAGGCCTGGCAGCCTGGCT
GGACAGTCAATGTGGAGGGCAGCGGGGAGCTGTTCCGGTGGAATGTTTCGGACCTAGGTGGCCTG
GGCTGTGGCCTGAAGAACAGGTCCTCAGAGGGCCCCAGCTCCCCTTCCGGGAAGCTCATGAGCCC
CAAGCTGTATGTGTGGGCCAAAGACCGCCCTGAGATCTGGGAGGGAGAGCCTCCGTGTCTCCCACC
GAGGGACAGCCTGAACCAGAGCCTCAGCCAGGACCTCACCATGGCCCCTGGCTCCACACTCTGGC
TGTCCTGTGGGGTACCCCCTGACTCTGTGTCCAGGGGCCCCCTCTCCTGGACCCATGTGCACCCCA
AGGGGCCTAAGTCATTGCTGAGCCTAGAGCTGAAGGACGATCGCCCGGCCAGAGATATGTGGGTAA
TGGAGACGGGTCTGTTGTTGCCCCGGGCCACAGCTCAAGACGCTGGAAAGTATTATTGTCACCGTG
GCAACCTGACCATGTCATTCCACCTGGAGATCACTGCTCGGCCAGGAGGAGGTGGGTCTGGAGGTG
GAGGATCTGGTGGAGGTGGGTCTGGAGGAGGTGGGTCTGACATGGCCCAGGTGCAGCTGCAGGAG
AGCGGCCCCGGCCTGGTGAAGCCCAGCGAGACCCTGAGCCTGACCTGCGTGGTGAGCGGCGGCA
GCATCAGCAGCAGCAACTGGTGGAGCTGGGTGAGGCAGCCCCCCGGCAAGGGCCTGGAGTGGATC
GGCGAGATCTACCACAGCGGCAGCCCCGACTACAACCCCAGCCTGAAGAGCAGGGTGACCATCAG
CGTGGACAAGAGCAGGAACCAGTTCAGCCTGAAGCTGAGCAGCGTGACCGCCGCCGACACCGCCG
TGTACTACTGCGCCAAGGTGAGCACCGGCGGCTTCTTCGACTACTGGGGCCAGGGCACCCTGGTG
ACCGTGAGCAGCGGCGGCGGCGGCAGCGGCGGCGGCGGCAGCGGCGGCGGCGGCAGCGAGATC
GAGCTGACCCAGAGCCCCAGCAGCCTGAGCGCCAGCGTGGGCGACAGGGTGACCATCACCTGCAG
GGCCAGCCAGAGCATCAGCAGCTACCTGAACTGGTACCAGCAGAAGCCCGGCAAGGCCCCCAAGC
TGCTGATCTACGCCGCCAGCAGCCTGCAGAGCGGCGTGCCCAGCAGGTTCAGCGGCAGCGGCAGC
GGCACCGACTTCACCCTGACCATCAGCAGCCTGCAGCCCGAGGACTTCGCCACCTACTACTGCCAG
CAGAGCTACAGCACCCCCCCCACCTTCGGCCCCGGCACCAAGGTGGAGATCAAGAGGACCCACCA
CCACCACCACCAC
Amino acid:
SEQ ID NO: 301
MPPPRLLFFLLFLTPMEVRPEEPLVVKVEEGDNAVLQCLKGTSDGPTQQLTWSRESPLKPFL
KLSLGLPGLGIHMRPLAIWLFIFNVSQQMGGFYLCQPGPPSEKAWQPGWTVNVEGSGELFR
WNVSDLGGLGCGLKNRSSEGPSSPSGKLMSPKLYVWAKDRPEIWEGEPPCLPPRDSLNQSLS
QDLTMAPGSTLWLSCGVPPDSVSRGPLSWTHVHPKGPKSLLSLELKDDRPARDMWVMETGL
LLPRATAQDAGKYYCHRGNLTMSFHLEITARPGGGGSGGGGSGGGGSGGGGSDMAQVQL
QESGPGLVKPSETLSLTCVVSGGSISSSNWWSWVRQPPGKGLEWIGEIYHSGSPDY
NPSLKSRVTISVDKSRNQFSLKLSSVTAADTAVYYCAKVSTGGFFDYWGQGTLVTV
SSGGGGSGGGGSGGGGSEIELTQSPSSLSASVGDRVTITCRASQSISSYLNWYQQKP
GKAPKLLIYAASSLQSGVPSRFSGSGSGTDFTLTISSLQPEDFATYYCQQSYSTPPTF

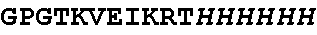

Construct #321
Nucleotide:
SEQ ID NO: 302
ATGCCACCTCCTCGCCTCCTCTTCTTCCTCCTCTTCCTCACCCCCATGGAAGTCAGGC
CCGAGGAGCCCCTGGTGGTGAAGGTGGAGGAGGGCGACACCGCCGTGCTGCCCTGC
CTGAAGGGCACCAGCGACGGCCCCACCCAGCAGCTGACCTGGAGCAGGGAGAGCCC
CCTGAAGCCCTTCCTGAAGTACAGCCTGGGCGTGCCCGGCCTGGGCGTGCACGTGA
GGCCCGACGCCATCAGCGTGGTGATCAGGAACGTGAGCCAGCAGATGGGCGGCTTC
TACCTGTGCCAGCCCGGCCCCCCCAGCGAGAAGGCCTGGCAGCCCGGCTGGACCGT
GAACGTGGAGGGCAGCGGCGAGCTGTTCAGGTGGAACGTGAGCGACCTGGGCGGCC
TGGGCTGCGGCCTGAAGAACAGGAGCAGCGAGGGCCCCAGCAGCCCCAGCGGCAA
GCTGATGAGCCCCAAGCTGTACGTGTGGGCCAAGGACAGGCCCGAGATCTGGGAGG
GCGAGCCCCCCTGCCTGCCCCCCAGGGACAGCCTGAACCAGAGCCTGAGCCAGGAC
CTGACCATGGCCCCCGGCAGCACCCTGTGGCTGAGCTGCGGCGTGCCCCCCGACAG
CGTGAGCAGGGGCCCCCTGAGCTGGACCCACGTGCACCCCAAGGGCCCCAAGAGCC
TGCTGAGCCTGGAGCTGAAGGACGACAGGCCCGCCAGGGAGATGATCGTGGACGAG
ACCGGCCTGCTGCTGCCCAGGGCCACCGCCCAGGACGCCGGCAAGTGGTACTGCAG
CAGGGGCAACGTGACCACCAGCTACCACCTGGAGATCACCGCCAGGCCCGTGAAGG
CCCACAGCGACCTGAGGACCGGCGGCTGGAAGGGAGGAGGTGGGTCTGGAGGTGG
AGGATCTGGTGGAGGTGGGTCTGGAGGAGGTGGATCCCAGGTGCAGCTGCAAGCGT
CTGGGGGAGGCTTGGTGCAGGCTGGGGGGTCTCTGAGACTCTCCTGTGCAGCCTCAG
GAAGCATCTTCGCTATTAATGAAATCAATCTTATGGGGTGGTACCGCCAGGCTCCAG
GGAAGCAGCGCGAGTTGGTCGCAGCTTGTGCTAGTGATGGCAACACATACTATGCG
GACTCCGTGAAGGGCCGATTCACCATCTCCAGAGACAACGCCGAGAAAACGGTGTA
TCTGCAGATGAACAACCTGAAACCTGACGACACAGCCGTCTATTACTGTGATGCGAA
TTCGAGGGGGAATTATTATTCGGGCCAGGGGACCCAGGTCACCGTTTCCTCAACTAG
TGGCCCGGGAGGCCAAGGCGCAGAACAAAAACTCATCTCAGAAGAGGATCTGGGC
GCACACCATCACCACCATCATGGCGCATCT
Amino acid:
SEQ ID NO: 303
MPPPRLLFFLLFLTPMEVRPEEPLVVKVEEGDTAVLPCLKGTSDGPTQQLTWSRESPLKPFL
KYSLGVPGLGVHVRPDAISVVIRNVSQQMGGFYLCQPGPPSEKAWQPGWTVNVEGSGELFR
WNVSDLGGLGCGLKNRSSEGPSSPSGKLMSPKLYVWAKDRPEIWEGEPPCLPPRDSLNQSLS
QDLTMAPGSTLWLSCGVPPDSVSRGPLSWTHVHPKGPKSLLSLELKDDRPAREMIVDETGLL
LPRATAQDAGKWYCSRGNVTTSYHLEITARPVKAHSDLRTGGWKGGGGSGGGGSGGGGSG
GGGSQVQLQASGGGLVQAGGSLRLSCAASGSIFAINEINLMGWYRQAPGKQRELV
AACASDGNTYYADSVKGRFTISRDNAEKTVYLQMNNLKPDDTAVYYCDANSRGNY

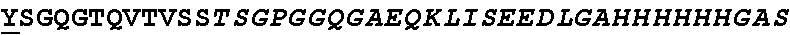

Construct #330
Nucleotide:
SEQ ID NO: 304
ATGGAGTTTGGGCTGAGCTGGGTTTTCCTCGTTGCTCTTTTTAGAGGTGTCCAGTGTC
AGGTGCAGCTGCAGGAGTCTGGGGGAGGCTTGGTGCAGGCTGGGGGGTCCCTTAGA
CTCTCCTGTGTAGCCTCTGGAAGCATCAGAAGTATCAATGTCATGGGCTGGTACCGC
CAGGCTCCAGGGAAGCAGCGCGAGTTGGTCGCAGCTTGTGCTAGTGATGGCAACAC
ATACTATGCGGACTCCGTGAAGGGCCGATTCACCATCTCCAGAGACAACGCCGAGA
AAACGGTGTATCTGCAGATGAACAACCTGAAACCTGACGACACAGCCGTCTATTACT
GTGATGCGAATTCGAGGGGGAATTATTATTCGGGCCAGGGGACCCAGGTCACCGTTT
CCTCAACTAGTGGCCCGGGAGGCCAAGGCGCAGGAGGAGGTGGGTCTGGAGGTGGA
GGATCTGGTGGAGGTGGGTCTGGAGGAGGTGGATCCCCCGAGGAACCTCTAGTGGT
GAAGGTGGAAGAGGGAGATACCGCTGCCCTGTGGTGCCTCAAGGGGACCTCAGATG
GCCCCACTCAGCAGCTGACCTGGTCTCGGGAGTCCCCGCTTAAACCCTTCTTAAAAT
ACAGCCTGGGGGTGCCAGGCCTGGGAGTGCACGTGAGGCCCGACGCCATCAGCGTG
GTTATCCGGAACGTCTCTCAACAGATGGGGGGCTTCTACCTGTGCCAGCCGGGGCCC
CCCTCTGAGAAGGCCTGGCAGCCTGGCTGGACAGTCAATGTGGAGGGCAGCGGGGA
GCTGTTCCGGTGGAATGTTTCGGACCTAGGTGGCCTGGGCTGTGGCCTGAAGAACAG
GTCCTCAGAGGGCCCCAGCTCCCCTTCCGGGAAGCTCATGAGCCCCAAGCTGTATGT
GTGGGCCAAAGACCGCCCTGAGATCTGGGAGGGAGAGCCTCCGTGTCTCCCACCGA
GGGACAGCCTGAACCAGAGCCTCAGCCGGGACCTCACCGTTGCCCCTGGCTCCACA
CTCTGGCTGTCCTGTGGGGTACCCCCTGACTCTGTGTCCAGGGGCCCCCTCTCCTGG
ACCCATGTGCACCCCAAGGGGCCTAAGTCATTGCTGAGCCTAGAGCTGAAGGACGA
TCGCCCGGCCAGAGATATGTGGGTAATGGGCACGAGCCTGATGTTGCCCCGGGCCA
CAGCTCAAGACGCTGGAAAGTGGTATTGTCACCGTGGCAACCTGACCATGTCATTCC
ACCTGGAGATCACTGCTCGGCCATCTAGACATCATCACCATCACCAT
Amino acid:
SEQ ID NO: 305
MEFGLSWVFLVALFRGVQCQVQLQESGGGLVQAGGSLRLSCVASGSIRSINVMGWY
RQAPGKORELVAACASDGNTYYADSVKGRFTISRDNAEKTVYLQMNNLKPDDTAV
YYCDANSRGNYYSGQGTQVTVSSTSGPGGQGAGGGGSGGGGSGGGGSGGGGSPEEP
LVVKVEEGDTAALWCLKGTSDGPTQQLTWSRESPLKPFLKYSLGVPGLGVHVRPDAISVVIRN
VSQQMGGFYLCQPGPPSEKAWQPGWTVNVEGSGELFRWNVSDLGGLGCGLKNRSSEGPSS
PSGKLMSPKLYVWAKDRPEIWEGEPPCLPPRDSLNQSLSRDLTVAPGSTLWLSCGVPPDSVSR
GPLSWTHVHPKGPKSLLSLELKDDRPARDMWVMGTSEMLPRATAQDAGKWYCHRGNLTMS

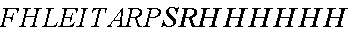

Construct #357
Nucleotide:
SEQ ID NO: 306
ATGGAGTTTGGGCTGAGCTGGGTTTTCCTCGTTGCTCTTTTTAGAGGTGTCCAGTGTG
ACATGGCCCAGGTGCAGCTGCAGGAGAGCGGCCCCGGCCTGGTGAAGCCCAGCGAG
ACCCTGAGCCTGACCTGCGTGGTGAGCGGCGGCAGCATCAGCAGCAGCAACTGGTG
GAGCTGGGTGAGGCAGCCCCCCGGCAAGGGCCTGGAGTGGATCGGCGAGATCTACC
ACAGCGGCAGCCCCGACTACAACCCCAGCCTGAAGAGCAGGGTGACCATCAGCGTG
GACAAGAGCAGGAACCAGTTCAGCCTGAAGCTGAGCAGCGTGACCGCCGCCGACAC
CGCCGTGTACTACTGCGCCAAGGTGAGCACCGGCGGCTTCTTCGACTACTGGGGCCA
GGGCACCCTGGTGACCGTGAGCAGCGGCGGCGGCGGCAGCGGCGGCGGCGGCAGC
GGCGGCGGCGGCAGCGAGATCGAGCTGACCCAGAGCCCCAGCAGCCTGAGCGCCA
GCGTGGGCGACAGGGTGACCATCACCTGCAGGGCCAGCCAGAGCATCAGCAGCTAC
CTGAACTGGTACCAGCAGAAGCCCGGCAAGGCCCCCAAGCTGCTGATCTACGCCGC
CAGCAGCCTGCAGAGCGGCGTGCCCAGCAGGTTCAGCGGCAGCGGCAGCGGCACCG
ACTTCACCCTGACCATCAGCAGCCTGCAGCCCGAGGACTTCGCCACCTACTACTGCC
AGCAGAGCTACAGCACCCCCCCCACCTTCGGCCCCGGCACCAAGGTGGAGATCAAG
AGGACCGGCGGCGGAGGATCTGGCGGAGGTGGAAGCGGAGGCGGAGGAAGCGGTG
GCGGCGGATCTCAGGTGCAGCTGCAGGAGTCTGGGGGAGGCTTGGTGCAGGCTGGG
GGGTCCCTTAGACTCTCCTGTGTAGCCTCTGGAAGCATCAGAAGTATCAATGTCATG
GGCTGGTACCGCCAGGCTCCAGGGAAGCAGCGCGAGTTGGTCGCAGCTTGTGCTAG
TGATGGCAACACATACTATGCGGACTCCGTGAAGGGCCGATTCACCATCTCCAGAG
ACAACGCCGAGAAAACGGTGTATCTGCAGATGAACAACCTGAAACCTGACGACACA
GCCGTCTATTACTGTGATGCGAATTCGAGGGGGAATTATTATTCGGGCCAGGGGACC
CAGGTCACCGTTTCCTCAACTAGTGGCCCGGGAGGCCAAGGTGCAGGAGGAGGGGG
GTCTGGGGGTGGAGGATCTGGTGGAGGTGGGTCTGGAGGAGGTGGATCCCCCGAGG
AACCTCTAGTGGTGAAGGTGGAAGAGGGAGATACCGCTGCCCTGTGGTGCCTCAAG
GGGACCTCAGATGGCCCCACTCAGCAGCTGACCTGGTCTCGGGAGTCCCCGCTTAAA
CCCTTCTTAAAATACAGCCTGGGGGTGCCAGGCCTGGGAGTGCACGTGAGGCCCGA
CGCCATCAGCGTGGTTATCCGGAACGTCTCTCAACAGATGGGGGGCTTCTACCTGTG
CCAGCCGGGGCCCCCCTCTGAGAAGGCCTGGCAGCCTGGCTGGACAGTCAATGTGG
AGGGCAGCGGGGAGCTGTTCCGGTGGAATGTTTCGGACCTAGGTGGCCTGGGCTGT
GGCCTGAAGAACAGGTCCTCAGAGGGCCCCAGCTCCCCTTCCGGGAAGCTCATGAG
CCCCAAGCTGTATGTGTGGGCCAAAGACCGCCCTGAGATCTGGGAGGGAGAGCCTC
CGTGTCTCCCACCGAGGGACAGCCTGAACCAGAGCCTCAGCCGGGACCTCACCGTT
GCCCCTGGCTCCACACTCTGGCTGTCCTGTGGGGTACCCCCTGACTCTGTGTCCAGG
GGCCCCCTCTCCTGGACCCATGTGCACCCCAAGGGGCCTAAGTCATTGCTGAGCCTA
GAGCTGAAGGACGATCGCCCGGCCAGAGATATGTGGGTAATGGGCACGAGCCTGAT
GTTGCCCCGGGCCACAGCTCAAGACGCTGGAAAGTGGTATTGTCACCGTGGCAACC
TGACCATGTCATTCCACCTGGAGATCACTGCTCGGCCATCTAGACATCATCACCATC
ACCAT
Amino acid:
SEQ ID NO: 307
MEFGLSWVFLVALFRGVQCDMAQVQLQESGPGLVKPSETLSLTCVVSGGSISSSNW
WSWVRQPPGKGLEWIGEIYHSGSPDYNPSLKSRVTISVDKSRNQFSLKLSSVTAADT
AVYYCAKVSTGGFFDYWGQGTLVTVSSGGGGSGGGGSGGGGSEIELTQSPSSLSAS
VGDRVTITCRASQSISSYLNWYQQKPGKAPKLLIYAASSLQSGVPSRFSGSGSGTDF
TLTISSLQPEDFATYYCQQSYSTPPTFGPGTKVEIKRTGGGGSGGGGSGGGGSGGGG
SQVQLQESGGGLVQAGGSLRLSCVASGSIRSINVMGWYRQAPGKQRELVAACASD
GNTYYADSVKGRFTISRDNAEKTVYLQMNNLKPDDTAVYYCDANSRGNYYSGQGT
QVTVSSTSGPGGQGAGGGGSGGGGSGGGGSGGGGSPEEPLVVKVEEGDTAALWCLKGT
SDGPTQQLTWSRESPLKPFLKYSLGVPGLGVHVRPDAISVVIRNVSQQMGGFYLCQPGPPSEK
AWQPGWTVNVEGSGELFRWNVSDLGGLGCGLKNRSSEGPSSPSGKLMSPKLYVWAKDRPEI
WEGEPPCLPPRDSLNQSLSRDLTVAPGSTLWLSCGVPPDSVSRGPLSWTHVHPKGPKSLLSLE

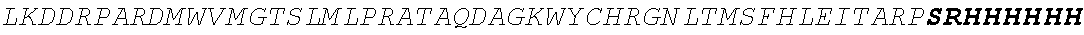

Construct #221
Nucleotide:
SEQ ID NO: 308
ATGAGGCTTCTGGTGCTTCTTTGGGGTTGCTTGCTGTTGCCCGGTTACGAAGCAGAC
ATCCAGATGACACAGACTACATCCTCCCTGTCTGCCTCTCTGGGAGACAGAGTCACC
ATCAGTTGCAGGGCAAGTCAGGACATTAGTAAATATTTAAATTGGTATCAGCAGAA
ACCAGATGGAACTGTTAAACTCCTGATCTACCATACATCAAGATTACACTCAGGAGT
CCCATCAAGGTTCAGTGGCAGTGGGTCTGGAACAGATTATTCTCTCACCATTAGCAA
CCTGGAGCAAGAAGATATTGCCACTTACTTTTGCCAACAAGGTAATACGCTTCCGTA
CACGTTCGGAGGGGGGACTAAGTTGGAAATAACAGGAGGCGGCGGGTCTGGAGGTG
GAGGATCTGGTGGTGGCGGGTCTGGAGGCGGCGGGTCTGAGGTGAAACTGCAGGAG
TCAGGACCTGGCCTGGTGGCGCCCTCACAGAGCCTGTCCGTCACATGCACTGTCTCA
GGGGTCTCATTACCCGACTATGGTGTAAGCTGGATTCGCCAGCCTCCACGAAAGGGT
CTGGAGTGGCTGGGAGTAATATGGGGTAGTGAAACCACATACTACAACTCAGCTCT
CAAATCCAGACTGACCATCATCAAGGACAACTCCAAGAGCCAAGTTTTCTTAAAAAT
GAACAGTCTGCAAACTGATGACACAGCCATTTACTACTGTGCCAAACATTATTACTA
CGGTGGTAGCTATGCTATGGACTACTGGGGTCAAGGAACCTCAGTCACCGTCTCCTC
AGACTACAAAGACGATGACGACAAGATTGAAGTTATGTATCCTCCTCCTTACCTAGA
CAATGAGAAGAGCAATGGAACCATTATCCATGTGAAAGGGAAACACCTTTGTCCAA
GTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTCCGCAGT
AAGAGGAGCAGGCTCCTGCACAGTGACTACATGAACATGACTCCCCGCCGCCCCGG
GCCCACCCGCAAGCATTACCAGCCCTATGCCCCACCACGCGACTTCGCAGCCTATCG
CTCCAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTTCA
CATGCAAGCCCTGCCCCCTCGCCGCGCGAAACGCAGCGGCAGCGGCGCGACCAACT
TTAGCCTGCTGAAACAGGCGGGCGATGTGGAAGAAAACCCGGGCCCGATGCCACCT
CCTCGCCTCCTCTTCTTCCTCCTCTTCCTCACCCCCATGGAAGTCAGGCCCGAGGAAC
CTCTAGTGGTGAAGGTGGAAGAGGGAGATAACGCTGTGCTGCAGTGCCTCAAGGGG
ACCTCAGATGGCCCCACTCAGCAGCTGACCTGGTCTCGGGAGTCCCCGCTTAAACCC
TTCTTAAAACTCAGCCTGGGGCTGCCAGGCCTGGGAATCCACATGAGGCCCCTGGCC
ATCTGGCTTTTCATCTTCAACGTCTCTCAACAGATGGGGGGCTTCTACCTGTGCCAGC
CGGGGCCCCCCTCTGAGAAGGCCTGGCAGCCTGGCTGGACAGTCAATGTGGAGGGC
AGCGGGGAGCTGTTCCGGTGGAATGTTTCGGACCTAGGTGGCCTGGGCTGTGGCCTG
AAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTC
TCCCACCGAGGGACAGCCTGAACCAGAGCCTCAGCCAGGACCTCACCATGGCCCCT
GGCTCCACACTCTGGCTGTCCTGTGGGGTACCCCCTGACTCTGTGTCCCGCGGCCCC
CTCTCCTGGACCCATGTGCACCCCAAGGGGCCTAAGTCATTGCTGAGCCTAGAGCTG
AAGGACGATCGCCCGGCCAGAGATATGTGGGTAATGGAGACGGGTCTGTTGTTGCC
CCGGGCCACAGCTCAAGACGCTGGAAAGTATTATTGTCACCGTGGCAACCTGACCA
TGTCATTCCACCTGGAGATCACTGCTCGGCCTGGCGGCGGCGGGTCTGGAGGTGGAG
GATCTGGTGGTGGCGGGTCTGGTGGCGGCGGGTCTGACATGGCCCAGGTGCAGCTG
CAGGAGAGCGGCCCCGGCCTGGTGAAGCCCAGCGAGACCCTGAGCCTGACCTGCGT
GGTGAGCGGCGGCAGCATCAGCAGCAGCAACTGGTGGAGCTGGGTGAGGCAGCCCC
CCGGCAAGGGCCTGGAGTGGATCGGCGAGATCTACCACAGCGGCAGCCCCGACTAC
AACCCCAGCCTGAAGAGCAGGGTGACCATCAGCGTGGACAAGAGCAGGAACCAGTT
CAGCCTGAAGCTGAGCAGCGTGACCGCCGCCGACACCGCCGTGTACTACTGCGCCA
AGGTGAGCACCGGCGGCTTCTTCGACTACTGGGGCCAGGGCACCCTGGTGACCGTG
AGCAGCGGCGGCGGCGGCAGCGGCGGCGGCGGCAGCGGCGGCGGCGGCAGCGAGA
TCGAGCTGACCCAGAGCCCCAGCAGCCTGAGCGCCAGCGTGGGCGACAGGGTGACC
ATCACCTGCAGGGCCAGCCAGAGCATCAGCAGCTACCTGAACTGGTACCAGCAGAA
GCCCGGCAAGGCCCCCAAGCTGCTGATCTACGCCGCCAGCAGCCTGCAGAGCGGCG
TGCCCAGCAGGTTCAGCGGCAGCGGCAGCGGCACCGACTTCACCCTGACCATCAGC
AGCCTGCAGCCCGAGGACTTCGCCACCTACTACTGCCAGCAGAGCTACAGCACCCC
CCCCACCTTCGGCCCCGGCACCAAGGTGGAGATCAAGAGGACCCACCACCACCACC
ACCAC
Amino acid:
SEQ ID NO: 309

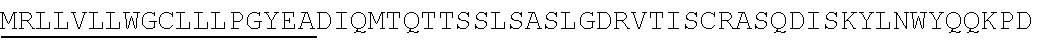

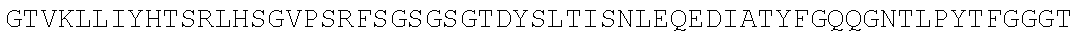

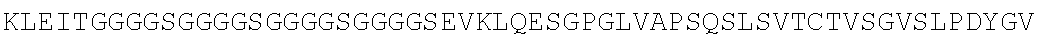

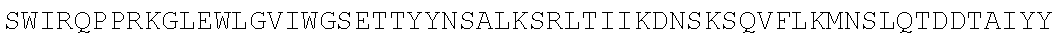

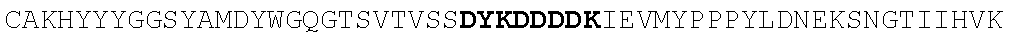

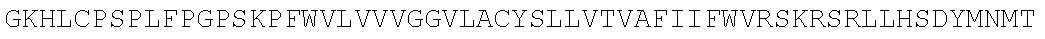

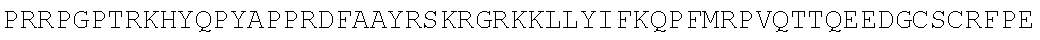

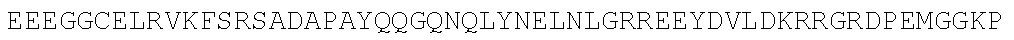

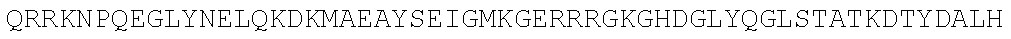

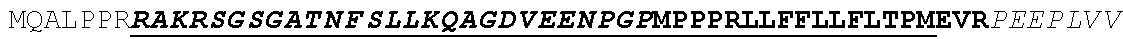

KVEEGDNAVLQCLKGTSDGPTQQLTWSRESPLKPFLKLSLGLPGLGIHMRPLAIWLFIFNVSQ
QMGGFYLCQPGPPSEKAWQPGWTVNVEGSGELFRWNVSDLGGLGCGLKNRSSEGPSSPSG
KLMSPKLYVWAKDRPEIWEGEPPCLPPRDSLNQSLSQDLTMAPGSTLWLSCGVPPDSVSRGP
LSWTHVHPKGPKSLLSLELKDDRPARDMWVMETGLLLPRATAQDAGKYYCHRGNLTMSFHL
EITARPGGGGSGGGGSGGGGSGGGGSDMAQVQLQESGPGLVKPSETLSLTCVVSGGS
ISSSNWWSWVRQPPGKGLEWIGEIYHSGSPDYNPSLKSRVTISVDKSRNQFSLKLSS
VTAADTAVYYCAKVSTGGFFDYWGOGTLVTVSSGGGGSGGGGSGGGGSEIELTQS
PSSLSASVGDRVTITCRASQSISSYLNWYQQKPGKAPKLLIYAASSLQSGVPSRFSGS

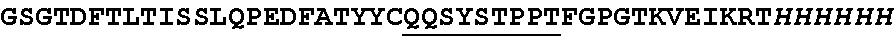

Construct #405
Nucleotide:
SEQ ID NO: 310
ATGAGGCTTCTGGTGCTTCTTTGGGGTTGCTTGCTGTTGCCCGGTTACGAAGCAGAC
ATCCAGATGACACAGACTACATCCTCCCTGTCTGCCTCTCTGGGAGACAGAGTCACC
ATCAGTTGCAGGGCAAGTCAGGACATTAGTAAATATTTAAATTGGTATCAGCAGAA
ACCAGATGGAACTGTTAAACTCCTGATCTACCATACATCAAGATTACACTCAGGAGT
CCCATCAAGGTTCAGTGGCAGTGGGTCTGGAACAGATTATTCTCTCACCATTAGCAA
CCTGGAGCAAGAAGATATTGCCACTTACTTTTGCCAACAAGGTAATACGCTTCCGTA
CACGTTCGGAGGGGGGACTAAGTTGGAAATAACAGGAGGCGGCGGGTCTGGAGGTG
GAGGATCTGGTGGTGGCGGGTCTGGAGGCGGCGGGTCTGAGGTGAAACTGCAGGAG
TCAGGACCTGGCCTGGTGGCGCCCTCACAGAGCCTGTCCGTCACATGCACTGTCTCA
GGGGTCTCATTACCCGACTATGGTGTAAGCTGGATTCGCCAGCCTCCACGAAAGGGT
CTGGAGTGGCTGGGAGTAATATGGGGTAGTGAAACCACATACTACAACTCAGCTCT
CAAATCCAGACTGACCATCATCAAGGACAACTCCAAGAGCCAAGTTTTCTTAAAAAT
GAACAGTCTGCAAACTGATGACACAGCCATTTACTACTGTGCCAAACATTATTACTA
CGGTGGTAGCTATGCTATGGACTACTGGGGTCAAGGAACCTCAGTCACCGTCTCCTC
AGACTACAAAGACGATGACGACAAGATTGAAGTTATGTATCCTCCTCCTTACCTAGA
CAATGAGAAGAGCAATGGAACCATTATCCATGTGAAAGGGAAACACCTTTGTCCAA
GTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTCCGCAGT
AAGAGGAGCAGGCTCCTGCACAGTGACTACATGAACATGACTCCCCGCCGCCCCGG
GCCCACCCGCAAGCATTACCAGCCCTATGCCCCACCACGCGACTTCGCAGCCTATCG
CTCCAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTTCA
CATGCAAGCCCTGCCCCCTCGCCGCGCGAAACGCAGCGGCAGCGGCGCGACCAACT
TTAGCCTGCTGAAACAGGCGGGCGATGTGGAAGAAAACCCGGGCCCGATGGAGTTT
GGGCTGAGCTGGGTTTTCCTCGTTGCTCTTTTTAGAGGTGTCCAGTGTGACATGGCCC
AGGTGCAGCTGCAGGAGAGCGGCCCCGGCCTGGTGAAGCCCAGCGAGACCCTGAGC
CTGACCTGCGTGGTGAGCGGCGGCAGCATCAGCAGCAGCAACTGGTGGAGCTGGGT
GAGGCAGCCCCCCGGCAAGGGCCTGGAGTGGATCGGCGAGATCTACCACAGCGGCA
GCCCCGACTACAACCCCAGCCTGAAGAGCAGGGTGACCATCAGCGTGGACAAGAGC
AGGAACCAGTTCAGCCTGAAGCTGAGCAGCGTGACCGCCGCCGACACCGCCGTGTA
CTACTGCGCCAAGGTGAGCACCGGCGGCTTCTTCGACTACTGGGGCCAGGGCACCCT
GGTGACCGTGAGCAGCGGCGGCGGCGGCAGCGGCGGCGGCGGCAGCGGCGGCGGC
GGCAGCGAGATCGAGCTGACCCAGAGCCCCAGCAGCCTGAGCGCCAGCGTGGGCGA
CAGGGTGACCATCACCTGCAGGGCCAGCCAGAGCATCAGCAGCTACCTGAACTGGT
ACCAGCAGAAGCCCGGCAAGGCCCCCAAGCTGCTGATCTACGCCGCCAGCAGCCTG
CAGAGCGGCGTGCCCAGCAGGTTCAGCGGCAGCGGCAGCGGCACCGACTTCACCCT
GACCATCAGCAGCCTGCAGCCCGAGGACTTCGCCACCTACTACTGCCAGCAGAGCT
ACAGCACCCCCCCCACCTTCGGCCCCGGCACCAAGGTGGAGATCAAGAGGACCGGC
GGCGGAGGATCTGGCGGAGGTGGAAGCGGAGGCGGAGGAAGCGGTGGCGGCGGAT
CTCAGGTGCAGCTGCAGGAGTCTGGGGGAGGCTTGGTGCAGGCTGGGGGGTCCCTT
AGACTCTCCTGTGTAGCCTCTGGAAGCATCAGAAGTATCAATGTCATGGGCTGGTAC
CGCCAGGCTCCAGGGAAGCAGCGCGAGTTGGTCGCAGCTTGTGCTAGTGATGGCAA
CACATACTATGCGGACTCCGTGAAGGGCCGATTCACCATCTCCAGAGACAACGCCG
AGAAAACGGTGTATCTGCAGATGAACAACCTGAAACCTGACGACACAGCCGTCTAT
TACTGTGATGCGAATTCGAGGGGGAATTATTATTCGGGCCAGGGGACCCAGGTCAC
CGTTTCCTCAACTAGTGGCCCGGGAGGCCAAGGTGCAGGAGGAGGGGGGTCTGGGG
GTGGAGGATCTGGTGGAGGTGGGTCTGGAGGAGGTGGATCCCCCGAGGAACCTCTA
GTGGTGAAGGTGGAAGAGGGAGATACCGCTGCCCTGTGGTGCCTCAAGGGGACCTC
AGATGGCCCCACTCAGCAGCTGACCTGGTCTCGGGAGTCCCCGCTTAAACCCTTCTT
AAAATACAGCCTGGGGGTGCCAGGCCTGGGAGTGCACGTGAGGCCCGACGCCATCA
GCGTGGTTATCCGGAACGTCTCTCAACAGATGGGGGGCTTCTACCTGTGCCAGCCGG
GGCCCCCCTCTGAGAAGGCCTGGCAGCCTGGCTGGACAGTCAATGTGGAGGGCAGC
GGGGAGCTGTTCCGGTGGAATGTTTCGGACCTAGGTGGCCTGGGCTGTGGCCTGAAG
AACAGGTCCTCAGAGGGCCCCAGCTCCCCTTCCGGGAAGCTCATGAGCCCCAAGCT
GTATGTGTGGGCCAAAGACCGCCCTGAGATCTGGGAGGGAGAGCCTCCGTGTCTCC
CACCGAGGGACAGCCTGAACCAGAGCCTCAGCCGGGACCTCACCGTTGCCCCTGGC
TCCACACTCTGGCTGTCCTGTGGGGTACCCCCTGACTCTGTGTCCAGGGGCCCCCTCT
CCTGGACCCATGTGCACCCCAAGGGGCCTAAGTCATTGCTGAGCCTAGAGCTGAAG
GACGATCGCCCGGCCAGAGATATGTGGGTAATGGGCACGAGCCTGATGTTGCCCCG
GGCCACAGCTCAAGACGCTGGAAAGTGGTATTGTCACCGTGGCAACCTGACCATGT
CATTCCACCTGGAGATCACTGCTCGGCCATCTAGACATCATCACCATCACCAT
Amino acid:
SEQ ID NO: 311

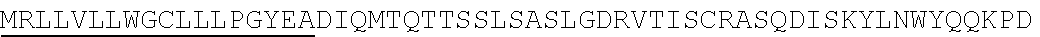

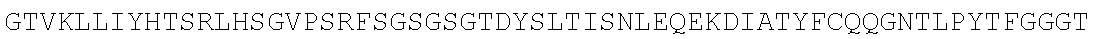

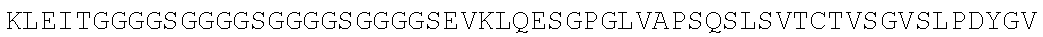

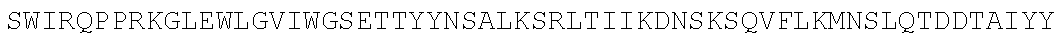

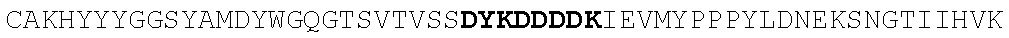

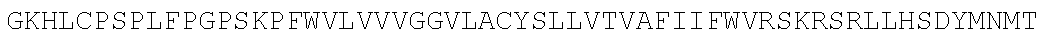

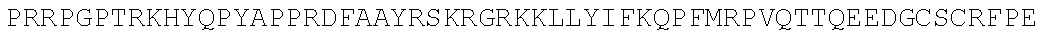

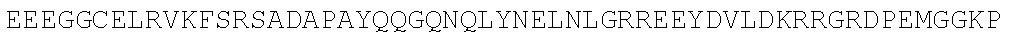

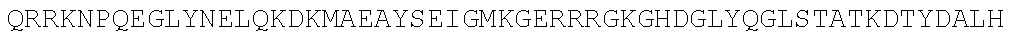

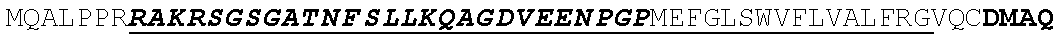

VQLQESGPGLVKPSETLSLTCVVSGGSISSSNWWSWVRQPPGKGLEWIGEIYHSGS
PDYNPSLKSRVTISVDKSRNQFSLKLSSVTAADTAVYYCAKVSTGGFFDYWGOGTL
VTVSSGGGGSGGGGSGGGGSEIELTQSPSSLSASVGDRVTITCRASQSISSYLNWYQ
QKPGKAPKLLIYAASSLQSGVPSRFSGSGSGTDFTLTISSLQPEDFATYYCQQSYSTP
PTFGPGTKVEIKRTGGGGSGGGGSGGGGSGGGGSQVQLQESGGGLVQAGGSLRLSC
VASGSIRSINVMGWYRQAPGKQRELVAACASDGNTYYADSVKGRFTISRDNAEKTV

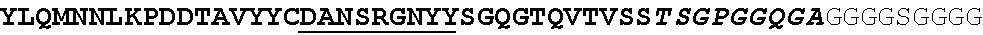

SGGGGSGGGGSPEEPLWKVEEGDTAALWCLKGTSDGPTQQLTWSRESPLKPFLKYSLGVP
GLGVHVRPDAISWIRNVSQQMGGFYLCQPGPPSEKAWQPGWTVNVEGSGELFRWNVSDLG
GLGCGLKNRSSEGPSSPSGKEMSPKLYVWAKDRPEIWEGEPPCLPPRDSLNQSLSRDLTVAP
GSTLWLSCGVPPDSVSRGPLSWTHVHPKGPKSLLSLELKDDRPARDMWVMGTSLMLPRATA

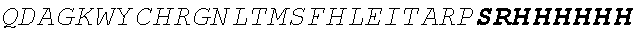

Construct #355
Nucleotide:
SEQ ID NO: 312
ATGAGGCTTCTGGTGCTTCTTTGGGGTTGCTTGCTGTTGCCCGGTTACGAAGCAGAC
ATCCAGATGACACAGACTACATCCTCCCTGTCTGCCTCTCTGGGAGACAGAGTCACC
ATCAGTTGCAGGGCAAGTCAGGACATTAGTAAATATTTAAATTGGTATCAGCAGAA
ACCAGATGGAACTGTTAAACTCCTGATCTACCATACATCAAGATTACACTCAGGAGT
CCCATCAAGGTTCAGTGGCAGTGGGTCTGGAACAGATTATTCTCTCACCATTAGCAA
CCTGGAGCAAGAAGATATTGCCACTTACTTTTGCCAACAAGGTAATACGCTTCCGTA
CACGTTCGGAGGGGGGACTAAGTTGGAAATAACAGGAGGCGGCGGGTCTGGAGGTG
GAGGATCTGGTGGTGGCGGGTCTGGAGGCGGCGGGTCTGAGGTGAAACTGCAGGAG
TCAGGACCTGGCCTGGTGGCGCCCTCACAGAGCCTGTCCGTCACATGCACTGTCTCA
GGGGTCTCATTACCCGACTATGGTGTAAGCTGGATTCGCCAGCCTCCACGAAAGGGT
CTGGAGTGGCTGGGAGTAATATGGGGTAGTGAAACCACATACTACAACTCAGCTCT
CAAATCCAGACTGACCATCATCAAGGACAACTCCAAGAGCCAAGTTTTCTTAAAAAT
GAACAGTCTGCAAACTGATGACACAGCCATTTACTACTGTGCCAAACATTATTACTA
CGGTGGTAGCTATGCTATGGACTACTGGGGTCAAGGAACCTCAGTCACCGTCTCCTC
AGACTACAAAGACGATGACGACAAGATTGAAGTTATGTATCCTCCTCCTTACCTAGA
CAATGAGAAGAGCAATGGAACCATTATCCATGTGAAAGGGAAACACCTTTGTCCAA
GTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTCCGCAGT
AAGAGGAGCAGGCTCCTGCACAGTGACTACATGAACATGACTCCCCGCCGCCCCGG
GCCCACCCGCAAGCATTACCAGCCCTATGCCCCACCACGCGACTTCGCAGCCTATCG
CTCCAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTTCA
CATGCAAGCCCTGCCCCCTCGCCGCGCGAAACGCAGCGGCAGCGGCGCGACCAACT
TTAGCCTGCTGAAACAGGCGGGCGATGTGGAAGAAAACCCGGGCCCGATGCCACCT
CCTCGCCTCCTCTTCTTCCTCCTCTTCCTCACCCCCATGGAAGTCAGGCCCGAGGAGC
CCCTGGTGGTGAAGGTGGAGGAGGGCGACACCGCCGTGCTGCCCTGCCTGAAGGGC
ACCAGCGACGGCCCCACCCAGCAGCTGACCTGGAGCAGGGAGAGCCCCCTGAAGCC
CTTCCTGAAGTACAGCCTGGGCGTGCCCGGCCTGGGCGTGCACGTGAGGCCCGACG
CCATCAGCGTGGTGATCAGGAACGTGAGCCAGCAGATGGGCGGCTTCTACCTGTGC
CAGCCCGGCCCCCCCAGCGAGAAGGCCTGGCAGCCCGGCTGGACCGTGAACGTGGA
GGGCAGCGGCGAGCTGTTCAGGTGGAACGTGAGCGACCTGGGCGGCCTGGGCTGCG
GCCTGAAGAACAGGAGCAGCGAGGGCCCCAGCAGCCCCAGCGGCAAGCTGATGAG
CCCCAAGCTGTACGTGTGGGCCAAGGACAGGCCCGAGATCTGGGAGGGCGAGCCCC
CCTGCCTGCCCCCCAGGGACAGCCTGAACCAGAGCCTGAGCCAGGACCTGACCATG
GCCCCCGGCAGCACCCTGTGGCTGAGCTGCGGCGTGCCCCCCGACAGCGTGAGCAG
GGGCCCCCTGAGCTGGACCCACGTGCACCCCAAGGGCCCCAAGAGCCTGCTGAGCC
TGGAGCTGAAGGACGACAGGCCCGCCAGGGAGATGATCGTGGACGAGACCGGCCT
GCTGCTGCCCAGGGCCACCGCCCAGGACGCCGGCAAGTGGTACTGCAGCAGGGGCA
ACGTGACCACCAGCTACCACCTGGAGATCACCGCCAGGCCCGTGAAGGCCCACAGC
GACCTGAGGACCGGCGGCTGGAAGGGAGGAGGTGGGTCTGGAGGTGGAGGATCTG
GTGGAGGTGGGTCTGGAGGAGGTGGATCCCAGGTGCAGCTGCAAGCGTCTGGGGGA
GGCTTGGTGCAGGCTGGGGGGTCTCTGAGACTCTCCTGTGCAGCCTCAGGAAGCATC
TTCGCTATTAATGAAATCAATCTTATGGGGTGGTACCGCCAGGCTCCAGGGAAGCAG
CGCGAGTTGGTCGCAGCTTGTGCTAGTGATGGCAACACATACTATGCGGACTCCGTG
AAGGGCCGATTCACCATCTCCAGAGACAACGCCGAGAAAACGGTGTATCTGCAGAT
GAACAACCTGAAACCTGACGACACAGCCGTCTATTACTGTGATGCGAATTCGAGGG
GGAATTATTATTCGGGCCAGGGGACCCAGGTCACCGTTTCCTCAACTAGTGGCCCGG
GAGGCCAAGGCGCAGAACAAAAACTCATCTCAGAAGAGGATCTGGGCGCACACCAT
CACCACCATCAT
Amino acid:
SEQ ID NO: 313

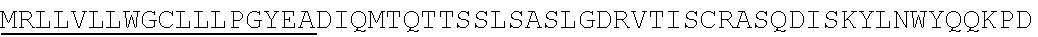

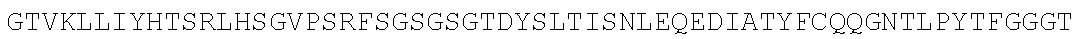

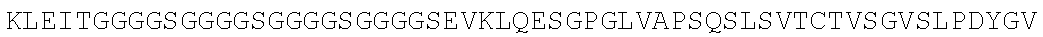

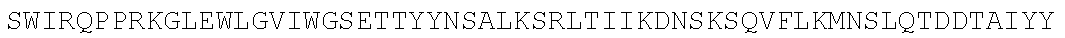

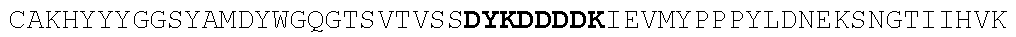

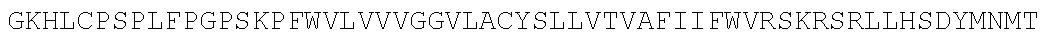

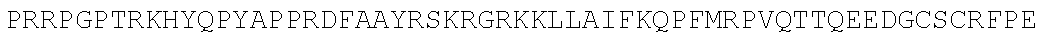

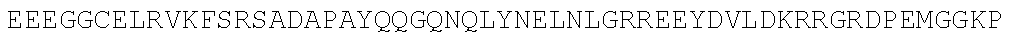

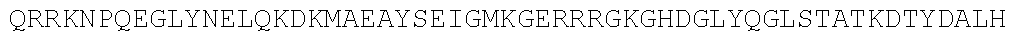

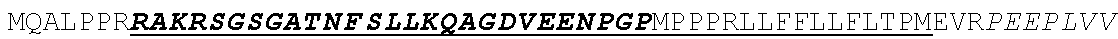

KVEEGDTAVLPCLKGTSDGPTQQLTWSRESPLKPFLKYSLGVPGLGVHVRPDAISWIRNVSQ
QMGGFYLCQPGPPSEKAWQPGWTVNVEGSGELFRWNVSDLGGLGCGLKNRSSEGPSSPSG
KLMSPKLYVWAKDRPEIWEGEPPCLPPRDSLNQSLSQDLTMAPGSTLWLSCGVPPDSVSRGP
LSWTHVHPKGPKSLLSLELKDDRPAREMIVDETGLLLPRATAQDAGKWYCSRGNVTTSYHLEI
TARPVKAHSDLRTGGWKGGGGSGGGGSGGGGSGGGGSQVQEQSGGGINQGGSLR
LSCAASGSIFAINEINLMGWYRQAPGKQRELVAACASDGNTYYADSVKGRFTISRD

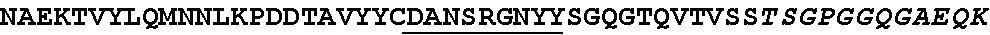

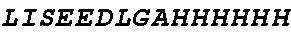

Construct #356
Nucleotide:
SEQ ID NO: 314
ATGAGGCTTCTGGTGCTTCTTTGGGGTTGCTTGCTGTTGCCCGGTTACGAAGCAGAC
ATCCAGATGACACAGACTACATCCTCCCTGTCTGCCTCTCTGGGAGACAGAGTCACC
ATCAGTTGCAGGGCAAGTCAGGACATTAGTAAATATTTAAATTGGTATCAGCAGAA
ACCAGATGGAACTGTTAAACTCCTGATCTACCATACATCAAGATTACACTCAGGAGT
CCCATCAAGGTTCAGTGGCAGTGGGTCTGGAACAGATTATTCTCTCACCATTAGCAA
CCTGGAGCAAGAAGATATTGCCACTTACTTTTGCCAACAAGGTAATACGCTTCCGTA
CACGTTCGGAGGGGGGACTAAGTTGGAAATAACAGGAGGCGGCGGGTCTGGAGGTG
GAGGATCTGGTGGTGGCGGGTCTGGAGGCGGCGGGTCTGAGGTGAAACTGCAGGAG
TCAGGACCTGGCCTGGTGGCGCCCTCACAGAGCCTGTCCGTCACATGCACTGTCTCA
GGGGTCTCATTACCCGACTATGGTGTAAGCTGGATTCGCCAGCCTCCACGAAAGGGT
CTGGAGTGGCTGGGAGTAATATGGGGTAGTGAAACCACATACTACAACTCAGCTCT
CAAATCCAGACTGACCATCATCAAGGACAACTCCAAGAGCCAAGTTTTCTTAAAAAT
GAACAGTCTGCAAACTGATGACACAGCCATTTACTACTGTGCCAAACATTATTACTA
CGGTGGTAGCTATGCTATGGACTACTGGGGTCAAGGAACCTCAGTCACCGTCTCCTC
AGACTACAAAGACGATGACGACAAGATTGAAGTTATGTATCCTCCTCCTTACCTAGA
CAATGAGAAGAGCAATGGAACCATTATCCATGTGAAAGGGAAACACCTTTGTCCAA
GTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTCCGCAGT
AAGAGGAGCAGGCTCCTGCACAGTGACTACATGAACATGACTCCCCGCCGCCCCGG
GCCCACCCGCAAGCATTACCAGCCCTATGCCCCACCACGCGACTTCGCAGCCTATCG
CTCCAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTTCA
CATGCAAGCCCTGCCCCCTCGCCGCGCGAAACGCAGCGGCAGCGGCGCGACCAACT
TTAGCCTGCTGAAACAGGCGGGCGATGTGGAAGAAAACCCGGGCCCGATGGAGTTT
GGGCTGAGCTGGGTTTTCCTCGTTGCTCTTTTTAGAGGTGTCCAGTGTCAGGTGCAGC
TGCAGGAGTCTGGGGGAGGCTTGGTGCAGGCTGGGGGGTCCCTTAGACTCTCCTGTG
TAGCCTCTGGAAGCATCAGAAGTATCAATGTCATGGGCTGGTACCGCCAGGCTCCAG
GGAAGCAGCGCGAGTTGGTCGCAGCTTGTGCTAGTGATGGCAACACATACTATGCG
GACTCCGTGAAGGGCCGATTCACCATCTCCAGAGACAACGCCGAGAAAACGGTGTA
TCTGCAGATGAACAACCTGAAACCTGACGACACAGCCGTCTATTACTGTGATGCGAA
TTCGAGGGGGAATTATTATTCGGGCCAGGGGACCCAGGTCACCGTTTCCTCAACTAG
TGGCCCGGGAGGCCAAGGCGCAGGAGGAGGTGGGTCTGGAGGTGGAGGATCTGGT
GGAGGTGGGTCTGGAGGAGGTGGATCCCCCGAGGAACCTCTAGTGGTGAAGGTGGA
AGAGGGAGATACCGCTGCCCTGTGGTGCCTCAAGGGGACCTCAGATGGCCCCACTC
AGCAGCTGACCTGGTCTCGGGAGTCCCCGCTTAAACCCTTCTTAAAATACAGCCTGG
GGGTGCCAGGCCTGGGAGTGCACGTGAGGCCCGACGCCATCAGCGTGGTTATCCGG
AACGTCTCTCAACAGATGGGGGGCTTCTACCTGTGCCAGCCGGGGCCCCCCTCTGAG
AAGGCCTGGCAGCCTGGCTGGACAGTCAATGTGGAGGGCAGCGGGGAGCTGTTCCG
GTGGAATGTTTCGGACCTAGGTGGCCTGGGCTGTGGCCTGAAGAACAGGTCCTCAG
AGGGCCCCAGCTCCCCTTCCGGGAAGCTCATGAGCCCCAAGCTGTATGTGTGGGCCA
AAGACCGCCCTGAGATCTGGGAGGGAGAGCCTCCGTGTCTCCCACCGAGGGACAGC
CTGAACCAGAGCCTCAGCCGGGACCTCACCGTTGCCCCTGGCTCCACACTCTGGCTG
TCCTGTGGGGTACCCCCTGACTCTGTGTCCAGGGGCCCCCTCTCCTGGACCCATGTG
CACCCCAAGGGGCCTAAGTCATTGCTGAGCCTAGAGCTGAAGGACGATCGCCCGGC
CAGAGATATGTGGGTAATGGGCACGAGCCTGATGTTGCCCCGGGCCACAGCTCAAG
ACGCTGGAAAGTGGTATTGTCACCGTGGCAACCTGACCATGTCATTCCACCTGGAGA
TCACTGCTCGGCCATCTAGACATCATCACCATCACCAT
Amino acid:
SEQ ID NO: 315

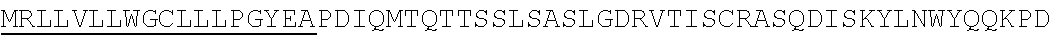

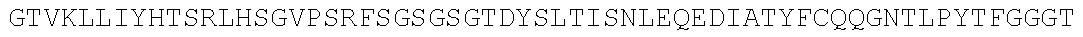

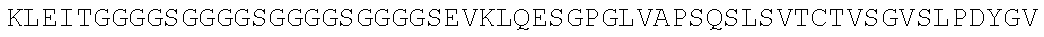

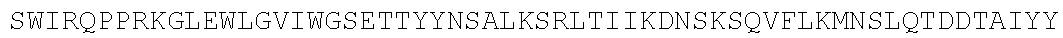

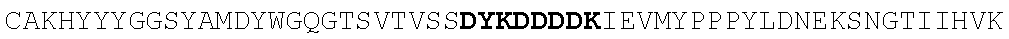

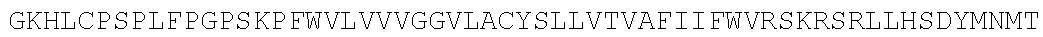

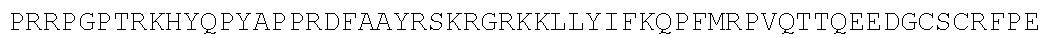

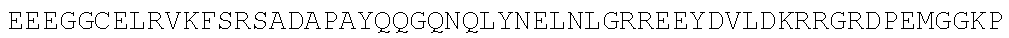

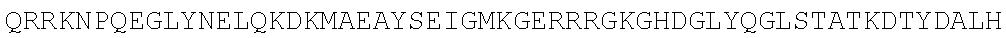

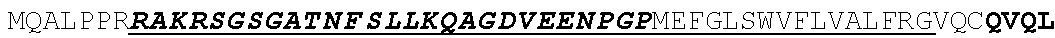

QESGGGLVOAGGSLRLSCVASGSIRSINVMGWYRQAPGKQRELVAACASDGNTYY
ADSVKGRFTISRDNAEKTVYLQMNNLKPDDTAVYYCDANSRGNYYSGQGTQVTVS

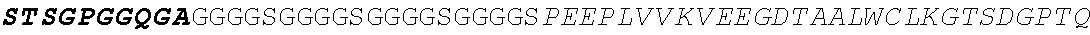

QLTWSRESPLKPFLKYSLGVPGLGVHVRPDAISWIRNVSQQMGGFYLCQPGPPSEKAWQPG
WTVNVEGSGELFRWNVSDLGGLGCGLKNRSSEGPSSPSGKLMSPKLYVWAKDRPEIWEGEP
PCLPPRDSLNQSLSRDLTVAPGSTLWLSCGVPPDSVSRGPLSWTHVHPKGPKSLLSLELKDDR

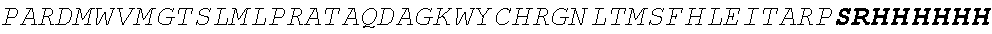

Construct #142
Nucleotide:
SEQ ID NO: 316
ATGAGGCTTCTGGTGCTTCTTTGGGGTTGCTTGCTGTTGCCCGGTTACGAAGCAGAC
ATCCAGATGACACAGACTACATCCTCCCTGTCTGCCTCTCTGGGAGACAGAGTCACC
ATCAGTTGCAGGGCAAGTCAGGACATTAGTAAATATTTAAATTGGTATCAGCAGAA
ACCAGATGGAACTGTTAAACTCCTGATCTACCATACATCAAGATTACACTCAGGAGT
CCCATCAAGGTTCAGTGGCAGTGGGTCTGGAACAGATTATTCTCTCACCATTAGCAA
CCTGGAGCAAGAAGATATTGCCACTTACTTTTGCCAACAAGGTAATACGCTTCCGTA
CACGTTCGGAGGGGGGACTAAGTTGGAAATAACAGGAGGCGGCGGGTCTGGAGGTG
GAGGATCTGGTGGTGGCGGGTCTGGAGGCGGCGGGTCTGAGGTGAAACTGCAGGAG
TCAGGACCTGGCCTGGTGGCGCCCTCACAGAGCCTGTCCGTCACATGCACTGTCTCA
GGGGTCTCATTACCCGACTATGGTGTAAGCTGGATTCGCCAGCCTCCACGAAAGGGT
CTGGAGTGGCTGGGAGTAATATGGGGTAGTGAAACCACATACTACAACTCAGCTCT
CAAATCCAGACTGACCATCATCAAGGACAACTCCAAGAGCCAAGTTTTCTTAAAAAT
GAACAGTCTGCAAACTGATGACACAGCCATTTACTACTGTGCCAAACATTATTACTA
CGGTGGTAGCTATGCTATGGACTACTGGGGTCAAGGAACCTCAGTCACCGTCTCCTC
AGACTACAAAGACGATGACGACAAGATTGAAGTTATGTATCCTCCTCCTTACCTAGA
CAATGAGAAGAGCAATGGAACCATTATCCATGTGAAAGGGAAACACCTTTGTCCAA
GTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTCCGCAGT
AAGAGGAGCAGGCTCCTGCACAGTGACTACATGAACATGACTCCCCGCCGCCCCGG
GCCCACCCGCAAGCATTACCAGCCCTATGCCCCACCACGCGACTTCGCAGCCTATCG
CTCCAAACGGGGCAGAAAGAAACTCCTGTATATATTCAAACAACCATTTATGAGAC
CAGTACAAACTACTCAAGAGGAAGATGGCTGTAGCTGCCGATTTCCAGAAGAAGAA
GAAGGAGGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAGAGGAGT
ACGATGTTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAAGATGG
CGGAGGCCTACAGTGAGATTGGGATGAAAGGCGAGCGCCGGAGGGGCAAGGGGCA
CGATGGCCTTTACCAGGGTCTCAGTACAGCCACCAAGGACACCTACGACGCCCTTCA
CATGCAAGCCCTGCCCCCTCGCCGCGCGAAACGCAGCGGCAGCGGCGCGACCAACT
TTAGCCTGCTGAAACAGGCGGGCGATGTGGAAGAAAACCCGGGCCCGATGCCACCT
CCTCGCCTCCTCTTCTTCCTCCTCTTCCTCACCCCCATGGAAGTCAGGCCCGAGGAAC
CTCTAGTGGTGAAGGTGGAAGAGGGAGATAACGCTGTGCTGCAGTGCCTCAAGGGG
ACCTCAGATGGCCCCACTCAGCAGCTGACCTGGTCTCGGGAGTCCCCGCTTAAACCC
TTCTTAAAACTCAGCCTGGGGCTGCCAGGCCTGGGAATCCACATGAGGCCCCTGGCC
ATCTGGCTTTTCATCTTCAACGTCTCTCAACAGATGGGGGGCTTCTACCTGTGCCAGC
CGGGGCCCCCCTCTGAGAAGGCCTGGCAGCCTGGCTGGACAGTCAATGTGGAGGGC
AGCGGGGAGCTGTTCCGGTGGAATGTTTCGGACCTAGGTGGCCTGGGCTGTGGCCTG
AAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTC
TCCCACCGAGGGACAGCCTGAACCAGAGCCTCAGCCAGGACCTCACCATGGCCCCT
GGCTCCACACTCTGGCTGTCCTGTGGGGTACCCCCTGACTCTGTGTCCCGCGGCCCC
CTCTCCTGGACCCATGTGCACCCCAAGGGGCCTAAGTCATTGCTGAGCCTAGAGCTG
AAGGACGATCGCCCGGCCAGAGATATGTGGGTAATGGAGACGGGTCTGTTGTTGCC
CCGGGCCACAGCTCAAGACGCTGGAAAGTATTATTGTCACCGTGGCAACCTGACCA
TGTCATTCCACCTGGAGATCACTGCTCGGCCTGGCGGCGGCGGGTCTGGAGGTGGAG
GATCTGGTGGTGGCGGGTCTGGTGGCGGCGGGTCTGAGGTGCAGCTGGTGGAGTCT
GGTGGTGGTCTTGTTCAACCTGGTGGTTCTCTTCGTCTTTCTTGTGCTGCTTCTGGTTT
TAATATTAAAGATACTTATATTCATTGGGTTCGTCAAGCTCCTGGTAAAGGTCTTGA
ATGGGTTGCTCGTATTTATCCTACTAATGGTTATACTCGTTATGCTGATTCTGTTAAA
GGTCGTTTTACTATTTCTGCTGATACTTCTAAAAATACTGCTTATCTTCAAATGAACT
CTCTTCGTGCTGAAGATACTGCTGTTTATTATTGTTCTCGTTGGGGTGGTGATGGTTT
TTATGCTATGGATTATTGGGGTCAAGGTACTCTTGTCACCGTCTCCTCAGCTAGCACC
GGGGGCGGCGGGTCTGGAGGTGGAGGATCTGGTGGCGGCGGGTCTGACATCCAGAT
GACCCAGTCTCCTTCTTCTCTTTCTGCTTCTGTTGGTGATCGTGTTACTATTACTTGTC
GTGCTTCTCAAGATGTTAATACTGCTGTTGCTTGGTATCAACAAAAACCTGGTAAAG
CTCCTAAACTTCTTATTTATTCTGCTTCTTTTCTTTATTCTGGTGTTCCTTCTCGTTTTT
CTGGTTCTCGTTCTGGTACTGATTTTACTCTTACTATTTCTTCTCTTCAACCTGAAGAT
TTTGCTACTTATTATTGTCAACAACATTATACTACTCCTCCTACTTTTGGTCAAGGTA
CCAAGGTGGAGATCAAACGTACGTCTAGACATCATCACCATCACCAT
Amino acid:
SEQ ID NO: 317

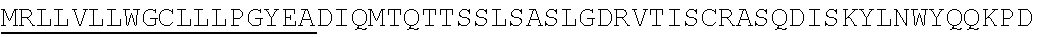

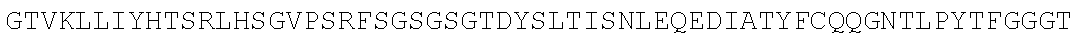

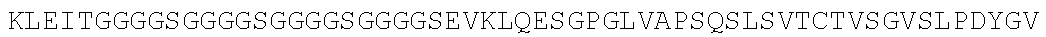

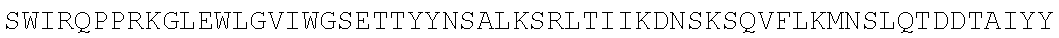

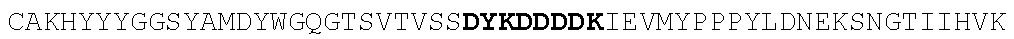

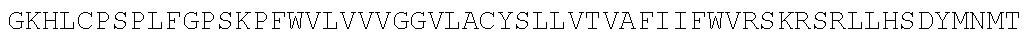

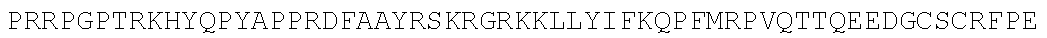

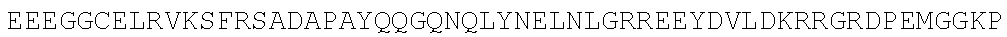

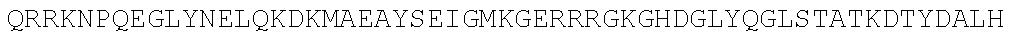

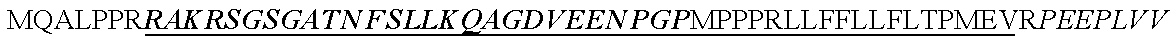

KVEEGDNAVLQCLKGTSDGPTQQLTWSRESPLKPFLKLSLGLPGLGIHMRPLAIWLFIFNVSQ
QMGGFYLCQPGPPSEKAWQPGWTVNVEGSGELFRWNVSDLGGLGCGLKNRSSEGPSSPSG
KLMSPKLYVWAKDRPEIWEGEPPCLPPRDSLNQSLSQDLTMAPGSTLWLSCGVPPDSVSRGP
LSWTHVHPKGPKSLLSLELKDDRPARDMWVMETGLLLPRATAQDAGKYYCHRGNLTMSFHL
EITARPGGGGSGGGGSGGGGSGGGGSEVQLVESGGGLVQPGGSLRLSCAASGFNIKD
TYIHWVRQAPGKGLEWVARIYPTNGYTRYADSVKGRFTISADTSKNTAYLQMNSL
RAEDTAVYYCSRWGGDGFYAMDYWGQGTLVTVSSASTGGGGSGGGGSGGGGSDIQ
MTQSPSSLSASVGDRVTITCRASQDVNTAVAWYQQKPGKAPKLLIYSASFLYSGVPS

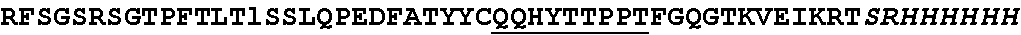

CDRs for Trastuzumab found in Akbarzadeh-Sharbaf et al. 2012 Adv Biomed Res. 1:21
Construct #260 (anti-CLEC12a CAR)
Nucleotide:
SEQ ID NO: 318
ATGGAGACCGACACCCTGCTGCTGTGGGTGCTGCTGCTGTGGGTGCCCGGCAGCACC
GGCGACATGGCCCAGGTGCAGCTGCAGGAGAGCGGCCCCGGCCTGGTGAAGCCCAG
CGAGACCCTGAGCCTGACCTGCGTGGTGAGCGGCGGCAGCATCAGCAGCAGCAACT
GGTGGAGCTGGGTGAGGCAGCCCCCCGGCAAGGGCCTGGAGTGGATCGGCGAGATC
TACCACAGCGGCAGCCCCGACTACAACCCCAGCCTGAAGAGCAGGGTGACCATCAG
CGTGGACAAGAGCAGGAACCAGTTCAGCCTGAAGCTGAGCAGCGTGACCGCCGCCG
ACACCGCCGTGTACTACTGCGCCAAGGTGAGCACCGGCGGCTTCTTCGACTACTGGG
GCCAGGGCACCCTGGTGACCGTGAGCAGCGGCGGCGGCGGCAGCGGCGGCGGCGG
CAGCGGCGGCGGCGGCAGCGAGATCGAGCTGACCCAGAGCCCCAGCAGCCTGAGC
GCCAGCGTGGGCGACAGGGTGACCATCACCTGCAGGGCCAGCCAGAGCATCAGCAG
CTACCTGAACTGGTACCAGCAGAAGCCCGGCAAGGCCCCCAAGCTGCTGATCTACG
CCGCCAGCAGCCTGCAGAGCGGCGTGCCCAGCAGGTTCAGCGGCAGCGGCAGCGGC
ACCGACTTCACCCTGACCATCAGCAGCCTGCAGCCCGAGGACTTCGCCACCTACTAC
TGCCAGCAGAGCTACAGCACCCCCCCCACCTTCGGCCCCGGCACCAAGGTGGAGAT
CAAGAGGACCTCTTCTGACTACAAAGACGATGACGACAAGATTGAAGTTATGTATC
CTCCTCCTTACCTAGACAATGAGAAGAGCAATGGAACCATTATCCATGTGAAAGGG
AAACACCTTTGTCCAAGTCCCCTATTTCCCGGACCTTCTAAGCCCTTTTGGGTGCTGG
TGGTGGTTGGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGAACATGA
CTCCCCGCCGCCCCGGGCCCACCCGCAAGCATTACCAGCCCTATGCCCCACCACGCG
ACTTCGCAGCCTATCGCTCCAAACGGGGCAGAAAGAAACTCCTGTATATATTCAAAC
AACCATTTATGAGACCAGTACAAACTACTCAAGAGGAAGATGGCTGTAGCTGCCGA
TTTCCAGAAGAAGAAGAAGGAGGATGTGAACTGAGAGTGAAGTTCAGCAGGAGCG
CAGACGCCCCCGCGTACCAGCAGGGCCAGAACCAGCTCTATAACGAGCTCAATCTA
GGACGAAGAGAGGAGTACGATGTTTTGGACAAGAGACGTGGCCGGGACCCTGAGAT
GGGGGGAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGAGCGCC
GGAGGGGCAAGGGGCACGATGGCCTTTACCAGGGTCTCAGTACAGCCACCAAGGAC
ACCTACGACGCCCTTCACATGCAGGCCCTGCCCCCTCGCTAA
Amino acid:
SEQ ID NO: 319
METDTLLLWVLLLWVPGSTGDMAQVQLQESGPGLVKPSETLSLTCVVSGGSISSSNW
WSWVRQPPGKGLEWIGEIYHSGSPDYNPSLKSRVTISVDKSRNQFSLKLSSVTAADT
AVYYCAKVSTGGFFDYWGQGTLVTVSSGGGGSGGGGSGGGGSEIELTQSPSSLSAS
VGDRVTITCRASQSISSYLNWYQQKPGKAPKLLIYAASSLOSGVPSRFSGSGSGTDF

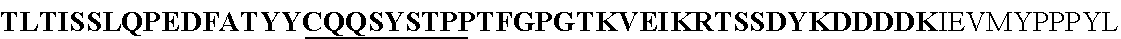

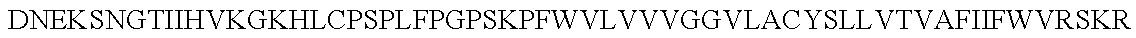

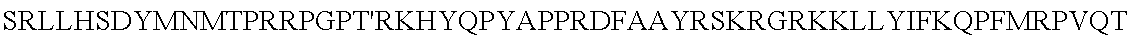

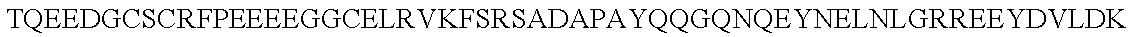

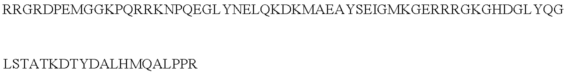

Construct #440
Nucleotide
SEQ ID NO: 320
ATGGAGTTTGGGCTGAGCTGGGTTTTCCTCGTTGCTCTTTTTAGAGGTGTCCAGTGTC
AGGTGCAGCTGGTGCAGAGCGGCGCCGAGGTGAAGAAGCCCGGCGAGAGCGTGAA
GGTGAGCTGCAAGGCCAGCGGCTACACCTTCACCAACTACGGCATGAACTGGGTGA
AGCAGGCCCCCGGCCAGGGCCTGGAGTGGATGGGCTGGATCAACACCTACACCGGC
GAGCCCACCTACGCCGACAAGTTCCAGGGCAGGGTGACCATGACCACCGACACCAG
CACCAGCACCGCCTACATGGAGATCAGGAACCTGGGCGGCGACGACACCGCCGTGT
ACTACTGCGCCAGGTGGAGCTGGAGCGACGGCTACTACGTGTACTTCGACTACTGGG
GCCAGGGCACCAGCGTGACCGTGAGCAGCGGCGGCGGCGGCAGCGGCGGCGGCGG
CAGCGGCGGCGGCGGCAGCGACATCGTGATGACCCAGAGCCCCGACAGCCTGACCG
TGAGCCTGGGCGAGAGGACCACCATCAACTGCAAGAGCAGCCAGAGCGTGCTGGAC
AGCAGCACCAACAAGAACAGCCTGGCCTGGTACCAGCAGAAGCCCGGCCAGCCCCC
CAAGCTGCTGCTGAGCTGGGCCAGCACCAGGGAGAGCGGCATCCCCGACAGGTTCA
GCGGCAGCGGCAGCGGCACCGACTTCACCCTGACCATCGACAGCCCCCAGCCCGAG
GACAGCGCCACCTACTACTGCCAGCAGAGCGCCCACTTCCCCATCACCTTCGGCCAG
GGCACCAGGCTGGAGATCAAGGGAGGAGGTGGGTCTGGAGGTGGAGGATCTGGTG
GAGGTGGGTCTGGAGGAGGTGGATCCCAGGTGCAGCTGCAGGAGTCTGGGGGAGGC
TTGGTGCAGGCTGGGGGGTCCCTTAGACTCTCCTGTGTAGCCTCTGGAAGCATCAGA
AGTATCAATGTCATGGGCTGGTACCGCCAGGCTCCAGGGAAGCAGCGCGAGTTGGT
CGCAGCTTGTGCTAGTGATGGCAACACATACTATGCGGACTCCGTGAAGGGCCGATT
CACCATCTCCAGAGACAACGCCGAGAAAACGGTGTATCTGCAGATGAACAACCTGA
AACCTGACGACACAGCCGTCTATTACTGTGATGCGAATTCGAGGGGGAATTATTATT
CGGGCCAGGGGACCCAGGTCACCGTTTCCTCAACTAGTGGCCCGGGAGGCCAAGGT
GCAGGAGGAGGGGGGTCTGGGGGTGGAGGATCTGGTGGAGGTGGGTCTGGAGGAG
GTGGATCCCCCGAGGAACCTCTAGTGGTGAAGGTGGAAGAGGGAGATACCGCTGCC
CTGTGGTGCCTCAAGGGGACCTCAGATGGCCCCACTCAGCAGCTGACCTGGTCTCGG
GAGTCCCCGCTTAAACCCTTCTTAAAATACAGCCTGGGGGTGCCAGGCCTGGGAGTG
CACGTGAGGCCCGACGCCATCAGCGTGGTTATCCGGAACGTCTCTCAACAGATGGG
GGGCTTCTACCTGTGCCAGCCGGGGCCCCCCTCTGAGAAGGCCTGGCAGCCTGGCTG
GACAGTCAATGTGGAGGGCAGCGGGGAGCTGTTCCGGTGGAATGTTTCGGACCTAG
GTGGCCTGGGCTGTGGCCTGAAGAACAGGTCCTCAGAGGGCCCCAGCTCCCCTTCCG
GGAAGCTCATGAGCCCCAAGCTGTATGTGTGGGCCAAAGACCGCCCTGAGATCTGG
GAGGGAGAGCCTCCGTGTCTCCCACCGAGGGACAGCCTGAACCAGAGCCTCAGCCG
GGACCTCACCGTTGCCCCTGGCTCCACACTCTGGCTGTCCTGTGGGGTACCCCCTGA
CTCTGTGTCCAGGGGCCCCCTCTCCTGGACCCATGTGCACCCCAAGGGGCCTAAGTC
ATTGCTGAGCCTAGAGCTGAAGGACGATCGCCCGGCCAGAGATATGTGGGTAATGG
GCACGAGCCTGATGTTGCCCCGGGCCACAGCTCAAGACGCTGGAAAGTGGTATTGT
CACCGTGGCAACCTGACCATGTCATTCCACCTGGAGATCACTGCTCGGCCATCTAGA
CATCATCACCATCACCAT
Amino Acid
SEQ ID NO: 321
MEFGLSWVFLVALFRGVQCQVQLVQSGAEVKKPGESVKVSCKASGYTFTNYGMNWV
KQAPGQGLEWMGWINTYTGEPTYADKFQGRVTMTTDTSTSTAYMEIRNLGGDDTAVY
YCARWSWSDGYYVYFDYWGQGTSVTVSSGGGGSGGGGSGGGGSDIVMTQSPDSLTVS
LGERTTINCKSSQSVLDSSTNKNSLAWYQQKPGQPPKLLLSWASTRESGIPDRFSGSGSG
TDFTLTIDSPQPEDSATYYCQQSAHFPITFGQGTRLEIKGGGGSGGGGSGGGGSGGGGSQ
VQLQESGGGLVQAGGSLRLSCVASGSIRSINVMGWYRQAPGKQRELVAACASDGNTYY
ADSVKGRFTISRDNAEKTVYLQMNNLKPDDTAVYYCDANSRGNYYSGQGTQVTVSSTS
GPGGQGAGGGGSGGGGSGGGGSGGGGSPEEPLVVKVEEGDTAALWCLKGTSDGPTQQ
LTWSRESPLKPFLKYSLGVPGLGVHVRPDAISVVIRNVSQQMGGFYLCQPGPPSEKAWQ
PGWTVNVEGSGELFRWNVSDLGGLGCGLKNRSSEGPSSPSGKLMSPKLYVWAKDRPEI
WEGEPPCLPPRDSLNQSLSRDLTVAPGSTLWLSCGVPPDSVSRGPLSWTHVHPKGPKSL
LSLELKDDRPARDMWVMGTSLMLPRATAQDAGKWYCHRGNLTMSFHLEITARPSRHH
HHHH
Construct #518
Nucleotide
SEQ ID NO: 322
ATGGAGTTTGGGCTGAGCTGGGTTTTCCTCGTTGCTCTTTTTAGAGGTGTCCAGTGTG
ACATGGCCCAGGTGCAGCTGCAGGAGAGCGGCCCCGGCCTGGTGAAGCCCAGCGAG
ACCCTGAGCCTGACCTGCGTGGTGAGCGGCGGCAGCATCAGCAGCAGCAACTGGTG
GAGCTGGGTGAGGCAGCCCCCCGGCAAGGGCCTGGAGTGGATCGGCGAGATCTACC
ACAGCGGCAGCCCCGACTACAACCCCAGCCTGAAGAGCAGGGTGACCATCAGCGTG
GACAAGAGCAGGAACCAGTTCAGCCTGAAGCTGAGCAGCGTGACCGCCGCCGACAC
CGCCGTGTACTACTGCGCCAAGGTGAGCACCGGCGGCTTCTTCGACTACTGGGGCCA
GGGCACCCTGGTGACCGTGAGCAGCGGCGGCGGCGGCAGCGGCGGCGGCGGCAGC
GGCGGCGGCGGCAGCGAGATCGAGCTGACCCAGAGCCCCAGCAGCCTGAGCGCCA
GCGTGGGCGACAGGGTGACCATCACCTGCAGGGCCAGCCAGAGCATCAGCAGCTAC
CTGAACTGGTACCAGCAGAAGCCCGGCAAGGCCCCCAAGCTGCTGATCTACGCCGC
CAGCAGCCTGCAGAGCGGCGTGCCCAGCAGGTTCAGCGGCAGCGGCAGCGGCACCG
ACTTCACCCTGACCATCAGCAGCCTGCAGCCCGAGGACTTCGCCACCTACTACTGCC
AGCAGAGCTACAGCACCCCCCCCACCTTCGGCCCCGGCACCAAGGTGGAGATCAAG
AGGACCGGCGGCGGAGGATCTGGCGGAGGTGGAAGCGGAGGCGGAGGAAGCGGTG
GCGGCGGATCTCAGGTGCAGCTGCAGGAGTCTGGGGGAGGCTTGGTGCAGGCTGGG
GGGTCCCTTAGACTCTCCTGTGTAGCCTCTGGAAGCATCAGAAGTATCAATGTCATG
GGCTGGTACCGCCAGGCTCCAGGGAAGCAGCGCGAGTTGGTCGCAGCTTGTGCTAG
TGATGGCAACACATACTATGCGGACTCCGTGAAGGGCCGATTCACCATCTCCAGAG
ACAACGCCGAGAAAACGGTGTATCTGCAGATGAACAACCTGAAACCTGACGACACA
GCCGTCTATTACTGTGATGCGAATTCGAGGGGGAATTATTATTCGGGCCAGGGGACC
CAGGTCACCGTTTCCTCAGGAGGAGGGGGGTCTGGGGGTGGAGGATCTGGTGGAGG
TGGGTCTGGAGGAGGTGGATCCCCCGAGGAACCTCTAGTGGTGAAGGTGGAAGAGG
GAGATACCGCTGCCCTGTGGTGCCTCAAGGGGACCTCAGATGGCCCCACTCAGCAG
CTGACCTGGTCTCGGGAGTCCCCGCTTAAACCCTTCTTAAAATACAGCCTGGGGGTG
CCAGGCCTGGGAGTGCACGTGAGGCCCGACGCCATCAGCGTGGTTATCCGGAACGT
CTCTCAACAGATGGGGGGCTTCTACCTGTGCCAGCCGGGGCCCCCCTCTGAGAAGGC
CTGGCAGCCTGGCTGGACAGTCAATGTGGAGGGCAGCGGGGAGCTGTTCCGGTGGA
ATGTTTCGGACCTAGGTGGCCTGGGCTGTGGCCTGAAGAACAGGTCCTCAGAGGGC
CCCAGCTCCCCTTCCGGGAAGCTCATGAGCCCCAAGCTGTATGTGTGGGCCAAAGAC
CGCCCTGAGATCTGGGAGGGAGAGCCTCCGTGTCTCCCACCGAGGGACAGCCTGAA
CCAGAGCCTCAGCCGGGACCTCACCGTTGCCCCTGGCTCCACACTCTGGCTGTCCTG
TGGGGTACCCCCTGACTCTGTGTCCAGGGGCCCCCTCTCCTGGACCCATGTGCACCC
CAAGGGGCCTAAGTCATTGCTGAGCCTAGAGCTGAAGGACGATCGCCCGGCCAGAG
ATATGTGGGTAATGGGCACGAGCCTGATGTTGCCCCGGGCCACAGCTCAAGACGCT
GGAAAGTGGTATTGTCACCGTGGCAACCTGACCATGTCATTCCACCTGGAGATCACT
GCTCGGCCAT
Amino Acid
SEQ ID NO: 323
MEFGLSWVFLVALFRGVQCDMAQVQLQESGPGLVKPSETLSLTCVVSGGSISSSNWWS
WVRQPPGKGLEWIGEIYHSGSPDYNPSLKSRVTISVDKSRNQFSLKLSSVTAADTAVYY
CAKVSTGGFFDYWGQGTLVTVSSGGGGSGGGGSGGGGSEIELTQSPSSLSASVGDRVTI
TCRASQSISSYLNWYQQKPGKAPKLLIYAASSLQSGVPSRFSGSGSGTDFTLTISSLQPED
FATYYCQQSYSTPPTFGPGTKVEIKRTGGGGSGGGGSGGGGSGGGGSQVQLQESGGGL
VQAGGSLRLSCVASGSIRSINVMGWYRQAPGKQRELVAACASDGNTYYADSVKGRFTI
SRDNAEKTVYLQMNNLKPDDTAVYYCDANSRGNYYSGQGTQVTVSSGGGGSGGGGS
GGGGSGGGGSPEEPLVVKVEEGDTAALWCLKGTSDGPTQQLTWSRESPLKPFLKYSLG
VPGLGVHVRPDAISVVIRNVSQQMGGFYLCQPGPPSEKAWQPGWTVNVEGSGELFRWN
VSDLGGLGCGLKNRSSEGPSSPSGKLMSPKLYVWAKDRPEIWEGEPPCLPPRDSLNQSLS
RDLTVAPGSTLWLSCGVPPDSVSRGPLSWTHVHPKGPKSLLSLELKDDRPARDMWVMG
TSLMLPRATAQDAGKWYCHRGNLTMSFHLEITARP
Construct #410
Nucleotide
SEQ ID NO: 324
ATGGAGTTTGGGCTGAGCTGGGTTTTCCTCGTTGCTCTTTTTAGAGGTGTCCAGTGTC
AGGTGCAGCTGGTGCAGAGCGGCGCCGAGGTGAAGAAGCCCGGCGAGAGCGTGAA
GGTGAGCTGCAAGGCCAGCGGCTACACCTTCACCAACTACGGCATGAACTGGGTGA
AGCAGGCCCCCGGCCAGGGCCTGGAGTGGATGGGCTGGATCAACACCTACACCGGC
GAGCCCACCTACGCCGACAAGTTCCAGGGCAGGGTGACCATGACCACCGACACCAG
CACCAGCACCGCCTACATGGAGATCAGGAACCTGGGCGGCGACGACACCGCCGTGT
ACTACTGCGCCAGGTGGAGCTGGAGCGACGGCTACTACGTGTACTTCGACTACTGGG
GCCAGGGCACCAGCGTGACCGTGAGCAGCGGCGGCGGCGGCAGCGGCGGCGGCGG
CAGCGGCGGCGGCGGCAGCGACATCGTGATGACCCAGAGCCCCGACAGCCTGACCG
TGAGCCTGGGCGAGAGGACCACCATCAACTGCAAGAGCAGCCAGAGCGTGCTGGAC
AGCAGCACCAACAAGAACAGCCTGGCCTGGTACCAGCAGAAGCCCGGCCAGCCCCC
CAAGCTGCTGCTGAGCTGGGCCAGCACCAGGGAGAGCGGCATCCCCGACAGGTTCA
GCGGCAGCGGCAGCGGCACCGACTTCACCCTGACCATCGACAGCCCCCAGCCCGAG
GACAGCGCCACCTACTACTGCCAGCAGAGCGCCCACTTCCCCATCACCTTCGGCCAG
GGCACCAGGCTGGAGATCAAGGGAGGAGGTGGGTCTGGAGGTGGAGGATCTGGTG
GAGGTGGGTCTGGAGGAGGTGGATCCCCCGAGGAACCTCTAGTGGTGAAGGTGGAA
GAGGGAGATACCGCTGCCCTGTGGTGCCTCAAGGGGACCTCAGATGGCCCCACTCA
GCAGCTGACCTGGTCTCGGGAGTCCCCGCTTAAACCCTTCTTAAAATACAGCCTGGG
GGTGCCAGGCCTGGGAGTGCACGTGAGGCCCGACGCCATCAGCGTGGTTATCCGGA
ACGTCTCTCAACAGATGGGGGGCTTCTACCTGTGCCAGCCGGGGCCCCCCTCTGAGA
AGGCCTGGCAGCCTGGCTGGACAGTCAATGTGGAGGGCAGCGGGGAGCTGTTCCGG
TGGAATGTTTCGGACCTAGGTGGCCTGGGCTGTGGCCTGAAGAACAGGTCCTCAGA
GGGCCCCAGCTCCCCTTCCGGGAAGCTCATGAGCCCCAAGCTGTATGTGTGGGCCAA
AGACCGCCCTGAGATCTGGGAGGGAGAGCCTCCGTGTCTCCCACCGAGGGACAGCC
TGAACCAGAGCCTCAGCCGGGACCTCACCGTTGCCCCTGGCTCCACACTCTGGCTGT
CCTGTGGGGTACCCCCTGACTCTGTGTCCAGGGGCCCCCTCTCCTGGACCCATGTGC
ACCCCAAGGGGCCTAAGTCATTGCTGAGCCTAGAGCTGAAGGACGATCGCCCGGCC
AGAGATATGTGGGTAATGGGCACGAGCCTGATGTTGCCCCGGGCCACAGCTCAAGA
CGCTGGAAAGTGGTATTGTCACCGTGGCAACCTGACCATGTCATTCCACCTGGAGAT
CACTGCTCGGCCATCTAGACATCATCACCATCACCAT
Amino Acid
SEQ ID NO: 325
MEFGLSWVFLVALFRGVQCQVQLVQSGAEVKKPGESVKVSCKASGYTFTNYGMNWV
KQAPGQGLEWMGWINTYTGEPTYADKFQGRVTMTTDTSTSTAYMEIRNLGGDDTAVY
YCARWSWSDGYYVYFDYWGQGTSVTVSSGGGGSGGGGSGGGGSDIVMTQSPDSLTVS
LGERTTINCKSSQSVLDSSTNKNSLAWYQQKPGQPPKLLLSWASTRESGIPDRFSGSGSG
TDFTLTIDSPQPEDSATYYCQQSAHFPITFGQGTRLEIKGGGGSGGGGSGGGGSGGGGSP
EEPLVVKVEEGDTAALWCLKGTSDGPTQQLTWSRESPLKPFLKYSLGVPGLGVHVRPD
AISVVIRNVSQQMGGFYLCQPGPPSEKAWQPGWTVNVEGSGELFRWNVSDLGGLGCGL
KNRSSEGPSSPSGKLMSPKLYVWAKDRPEIWEGEPPCLPPRDSLNQSLSRDLTVAPGSTL
WLSCGVPPDSVSRGPLSWTHVHPKGPKSLLSLELKDDRPARDMWVMGTSLMLPRATA
QDAGKWYCHRGNLTMSFHLEITARPSRHHHHHH
Construct # 468
Nucleotide
SEQ ID NO: 326
ATGAGGCTTCTGGTGCTTCTTTGGGGTTGCTTGCTGTTGCCCGGTTACGAAGCAGAC
ATCCAGATGACACAGACTACATCCTCCCTGTCTGCCTCTCTGGGAGACAGAGTCACC
ATCAGTTGCAGGGCAAGTCAGGACATTAGTAAATATTTAAATTGGTATCAGCAGAA
ACCAGATGGAACTGTTAAACTCCTGATCTACCATACATCAAGATTACACTCAGGAGT
CCCATCAAGGTTCAGTGGCAGTGGGTCTGGAACAGATTATTCTCTCACCATTAGCAA
CCTGGAGCAAGAAGATATTGCCACTTACTTTTGCCAACAAGGTAATACGCTTCCGTA
CACGTTCGGAGGGGGGACTAAGTTGGAAATAACAGGAGGCGGCGGGTCTGGAGGTG
GAGGATCTGGTGGTGGCGGGTCTGGAGGCGGCGGGTCTGAGGTGAAACTGCAGGAG
TCAGGACCTGGCCTGGTGGCGCCCTCACAGAGCCTGTCCGTCACATGCACTGTCTCA
GGGGTCTCATTACCCGACTATGGTGTAAGCTGGATTCGCCAGCCTCCACGAAAGGGT
CTGGAGTGGCTGGGAGTAATATGGGGTAGTGAAACCACATACTACAACTCAGCTCT
CAAATCCAGACTGACCATCATCAAGGACAACTCCAAGAGCCAAGTTTTCTTAAAAAT
GAACAGTCTGCAAACTGATGACACAGCCATTTACTACTGTGCCAAACATTATTACTA
CGGTGGTAGCTATGCTATGGACTACTGGGGTCAAGGAACCTCAGTCACCGTCTCCTC
AATTGAAGTTATGTATCCTCCTCCTTACCTAGACAATGAGAAGAGCAATGGAACCAT
TATCCATGTGAAAGGGAAACACCTTTGTCCAAGTCCCCTATTTCCCGGACCTTCTAA
GCCCTTTTGGGTGCTGGTGGTGGTTGGTGGAGTCCTGGCTTGCTATAGCTTGCTAGTA
ACAGTGGCCTTTATTATTTTCTGGGTCCGCAGTAAGAGGAGCAGGCTCCTGCACAGT
GACTACATGAACATGACTCCCCGCCGCCCCGGGCCCACCCGCAAGCATTACCAGCC
CTATGCCCCACCACGCGACTTCGCAGCCTATCGCTCCAAACGGGGCAGAAAGAAAC
TCCTGTATATATTCAAACAACCATTTATGAGACCAGTACAAACTACTCAAGAGGAAG
ATGGCTGTAGCTGCCGATTTCCAGAAGAAGAAGAAGGAGGATGTGAACTGAGAGTG
AAGTTCAGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGAGACGTG
GCCGGGACCCTGAGATGGGGGGAAAGCCGCAGAGAAGGAAGAACCCTCAGGAAGG
CCTGTACAATGAACTGCAGAAAGATAAGATGGCGGAGGCCTACAGTGAGATTGGGA
TGAAAGGCGAGCGCCGGAGGGGCAAGGGGCACGATGGCCTTTACCAGGGTCTCAGT
ACAGCCACCAAGGACACCTACGACGCCCTTCACATGCAAGCCCTGCCCCCTCGCCGC
GCGAAACGCAGCGGCAGCGGCGCGACCAACTTTAGCCTGCTGAAACAGGCGGGCGA
TGTGGAAGAAAACCCGGGCCCGATGGAGTTTGGGCTGAGCTGGGTTTTCCTCGTTGC
TCTTTTTAGAGGTGTCCAGTGTGACATGGCCCAGGTGCAGCTGCAGGAGAGCGGCCC
CGGCCTGGTGAAGCCCAGCGAGACCCTGAGCCTGACCTGCGTGGTGAGCGGCGGCA
GCATCAGCAGCAGCAACTGGTGGAGCTGGGTGAGGCAGCCCCCCGGCAAGGGCCTG
GAGTGGATCGGCGAGATCTACCACAGCGGCAGCCCCGACTACAACCCCAGCCTGAA
GAGCAGGGTGACCATCAGCGTGGACAAGAGCAGGAACCAGTTCAGCCTGAAGCTGA
GCAGCGTGACCGCCGCCGACACCGCCGTGTACTACTGCGCCAAGGTGAGCACCGGC
GGCTTCTTCGACTACTGGGGCCAGGGCACCCTGGTGACCGTGAGCAGCGGCGGCGG
CGGCAGCGGCGGCGGCGGCAGCGGCGGCGGCGGCAGCGAGATCGAGCTGACCCAG
AGCCCCAGCAGCCTGAGCGCCAGCGTGGGCGACAGGGTGACCATCACCTGCAGGGC
CAGCCAGAGCATCAGCAGCTACCTGAACTGGTACCAGCAGAAGCCCGGCAAGGCCC
CCAAGCTGCTGATCTACGCCGCCAGCAGCCTGCAGAGCGGCGTGCCCAGCAGGTTC
AGCGGCAGCGGCAGCGGCACCGACTTCACCCTGACCATCAGCAGCCTGCAGCCCGA
GGACTTCGCCACCTACTACTGCCAGCAGAGCTACAGCACCCCCCCCACCTTCGGCCC
CGGCACCAAGGTGGAGATCAAGAGGACCGGCGGCGGAGGATCTGGCGGAGGTGGA
AGCGGAGGCGGAGGAAGCGGTGGCGGCGGATCTCAGGTGCAGCTGCAGGAGTCTG
GGGGAGGCTTGGTGCAGGCTGGGGGGTCCCTTAGACTCTCCTGTGTAGCCTCTGGAA
GCATCAGAAGTATCAATGTCATGGGCTGGTACCGCCAGGCTCCAGGGAAGCAGCGC
GAGTTGGTCGCAGCTTGTGCTAGTGATGGCAACACATACTATGCGGACTCCGTGAAG
GGCCGATTCACCATCTCCAGAGACAACGCCGAGAAAACGGTGTATCTGCAGATGAA
CAACCTGAAACCTGACGACACAGCCGTCTATTACTGTGATGCGAATTCGAGGGGGA
ATTATTATTCGGGCCAGGGGACCCAGGTCACCGTTTCCTCAGGAGGAGGGGGGTCTG
GGGGTGGAGGATCTGGTGGAGGTGGGTCTGGAGGAGGTGGATCCCCCGAGGAACCT
CTAGTGGTGAAGGTGGAAGAGGGAGATACCGCTGCCCTGTGGTGCCTCAAGGGGAC
CTCAGATGGCCCCACTCAGCAGCTGACCTGGTCTCGGGAGTCCCCGCTTAAACCCTT
CTTAAAATACAGCCTGGGGGTGCCAGGCCTGGGAGTGCACGTGAGGCCCGACGCCA
TCAGCGTGGTTATCCGGAACGTCTCTCAACAGATGGGGGGCTTCTACCTGTGCCAGC
CGGGGCCCCCCTCTGAGAAGGCCTGGCAGCCTGGCTGGACAGTCAATGTGGAGGGC
AGCGGGGAGCTGTTCCGGTGGAATGTTTCGGACCTAGGTGGCCTGGGCTGTGGCCTG
AAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTC
TCCCACCGAGGGACAGCCTGAACCAGAGCCTCAGCCGGGACCTCACCGTTGCCCCT
GGCTCCACACTCTGGCTGTCCTGTGGGGTACCCCCTGACTCTGTGTCCAGGGGCCCC
CTCTCCTGGACCCATGTGCACCCCAAGGGGCCTAAGTCATTGCTGAGCCTAGAGCTG
AAGGACGATCGCCCGGCCAGAGATATGTGGGTAATGGGCACGAGCCTGATGTTGCC
CCGGGCCACAGCTCAAGACGCTGGAAAGTGGTATTGTCACCGTGGCAACCTGACCA
TGTCATTCCACCTGGAGATCACTGCTCGGCCA
Amino Acid
SEQ ID NO: 327
MRLLVLLWGCLLLPGYEADIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGT
KLEITGGGGSGGGGSGGGGSGGGGSEVKLQESGPGLVAPSQSLSVTCTVSGVSLPDYGV
SWIRQPPRKGLEWLGVIWGSETTYYNSALKSRLTIIKDNSKSQVFLKMNSLQTDDTAIYY
CAKHYYYGGSYAMDYWGQGTSVTVSSIEVMYPPPYLDNEKSNGTIIHVKGKHLCPSPL
FPGPSKPFWVLVVVGGVLACYSLLVTVAFIIFWVRSKRSRLLHSDYMNMTPRRPGPTRK
HYQPYAPPRDFAAYRSKRGRKKLLYIFKQPFMRPVQTTQEEDGCSCRFPEEEEGGCELR
VKFSRSADAPAYQQGQNQLYNELNLGRREEYDVLDKRRGRDPEMGGKPQRRKNPQEG
LYNELQKDKMAEAYSEIGMKGERRRGKGHDGLYQGLSTATKDTYDALHMQALPPRRA
KRSGSGATNFSLLKQAGDVEENPGPMEFGLSWVFLVALFRGVQCDMAQVQLQESGPGL
VKPSETLSLTCVVSGGSISSSNWWSWVRQPPGKGLEWIGEIYHSGSPDYNPSLKSRVTIS
VDKSRNQFSLKLSSVTAADTAVYYCAKVSTGGFFDYWGQGTLVTVSSGGGGSGGGGS
GGGGSEIELTQSPSSLSASVGDRVTITCRASQSISSYLNWYQQKPGKAPKLLIYAASSLQS
GVPSRFSGSGSGTDFTLTISSLQPEDFATYYCQQSYSTPPTFGPGTKVEIKRTGGGGSGGG
GSGGGGSGGGGSQVQLQESGGGLVQAGGSLRLSCVASGSIRSINVMGWYRQAPGKQRE
LVAACASDGNTYYADSVKGRFTISRDNAEKTVYLQMNNLKPDDTAVYYCDANSRGNY
YSGQGTQVTVSSGGGGSGGGGSGGGGSGGGGSPEEPLVVKVEEGDTAALWCLKGTSD
GPTQQLTWSRESPLKPFLKYSLGVPGLGVHVRPDAISVVIRNVSQQMGGFYLCQPGPPSE
KAWQPGWTVNVEGSGELFRWNVSDLGGLGCGLKNRSSEGPSSPSGKLMSPKLYVWAK
DRPEIWEGEPPCLPPRDSLNQSLSRDLTVAPGSTLWLSCGVPPDSVSRGPLSWTHVHPKG
PKSLLSLELKDDRPARDMWVMGTSLMLPRATAQDAGKWYCHRGNLTMSFHLEITARP
SEQ ID NO: 328
MWIDFFTYSSMSEEVTYADLQFQNSSEMEKIPEIGKFGEKAPPAPSHVWRPAALFLTLLC
LLLLIGLGVLASMFHVTLKIEMKKMNKLQNISEELQRNISLQLMSNMNISNKIRNLSTTL
QTIATKLCRELYSKEQEHKCKPCPRRWIWHKDSCYFLSDDVQTWQESKMACAAQNASL
LKINNKNALEFIKSQSRSYDYWLGLSPEEDSTRGMRVDNIINSSAWVIRNAPDLNNMYC
GYINRLYVQYYHCTYKKRMICEKMANPVQLGSTYFREA
SEQ ID NO: 329
MWIDFFTYSSMSEEVTYADLQFQNSSEMEKIPEIGKFGEKAPPAPSHVWRPAALFLTLLC
LLLLIGLGVLASMFHVTLKIEMKKMNKLQNISEELQRNISLQLMSNMNISNKIRNLSTTL
QTIATKLCRELYSKEQEHKCKPCPRRWIWHKDSCYFLSDDVQTWQESKMACAAQNASL
LKINNKNALEFIKSQSRSYDYWLGLSPEEDSTRGMRVDNIINSSAWVIRNAPDLNNMYC
GYINRLYVQYYHCTYKQRMICEKMANPVQLGSTYFREA
Construct # 408
Nucleotide
SEQ ID NO: 330
ATGGAGTTTGGGCTGAGCTGGGTTTTCCTCGTTGCTCTTTTTAGAGGTGTCCAGTGTC
AGGTGCAGCTGCAGGAGTCTGGGGGAGGCTTGGTGCAGGCTGGGGGGTCCCTTAGA
CTCTCCTGTGTAGCCTCTGGAAGCATCAGAAGTATCAATGTCATGGGCTGGTACCGC
CAGGCTCCAGGGAAGCAGCGCGAGTTGGTCGCAGCTTGTGCTAGTGATGGCAACAC
ATACTATGCGGACTCCGTGAAGGGCCGATTCACCATCTCCAGAGACAACGCCGAGA
AAACGGTGTATCTGCAGATGAACAACCTGAAACCTGACGACACAGCCGTCTATTACT
GTGATGCGAATTCGAGGGGGAATTATTATTCGGGCCAGGGGACCCAGGTCACCGTTT
CCTCAACTAGTGGCCCGGGAGGCCAAGGCGCAGACTACAAAGACGATGACGACAA
GATTGAAGTTATGTATCCTCCTCCTTACCTAGACAATGAGAAGAGCAATGGAACCAT
TATCCATGTGAAAGGGAAACACCTTTGTCCAAGTCCCCTATTTCCCGGACCTTCTAA
GCCCTTTTGGGTGCTGGTGGTGGTTGGTGGAGTCCTGGCTTGCTATAGCTTGCTAGTA
ACAGTGGCCTTTATTATTTTCTGGGTCCGCAGTAAGAGGAGCAGGCTCCTGCACAGT
GACTACATGAACATGACTCCCCGCCGCCCCGGGCCCACCCGCAAGCATTACCAGCC
CTATGCCCCACCACGCGACTTCGCAGCCTATCGCTCCAAACGGGGCAGAAAGAAAC
TCCTGTATATATTCAAACAACCATTTATGAGACCAGTACAAACTACTCAAGAGGAAG
ATGGCTGTAGCTGCCGATTTCCAGAAGAAGAAGAAGGAGGATGTGAACTGAGAGTG
AAGTTCAGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGAGACGTG
GCCGGGACCCTGAGATGGGGGGAAAGCCGCAGAGAAGGAAGAACCCTCAGGAAGG
CCTGTACAATGAACTGCAGAAAGATAAGATGGCGGAGGCCTACAGTGAGATTGGGA
TGAAAGGCGAGCGCCGGAGGGGCAAGGGGCACGATGGCCTTTACCAGGGTCTCAGT
ACAGCCACCAAGGACACCTACGACGCCCTTCACATGCAAGCCCTGCCCCCTCGC
Amino Acid
SEQ ID NO: 331
MEFGLSWVFLVALFRGVQCQVQLQESGGGLVQAGGSLRLSCVASGSIRSINVMGWYRQ
APGKQRELVAACASDGNTYYADSVKGRFTISRDNAEKTVYLQMNNLKPDDTAVYYCD
ANSRGNYYSGQGTQVTVSSTSGPGGQGADYKDDDDKIEVMYPPPYLDNEKSNGTIIHV
KGKHLCPSPLFPGPSKPFWVLVVVGGVLACYSLLVTVAFIIFWVRSKRSRLLHSDYMNM
TPRRPGPTRKHYQPYAPPRDFAAYRSKRGRKKLLYIFKQPFMRPVQTTQEEDGCSCRFP
EEEEGGCELRVKFSRSADAPAYQQGQNQLYNELNLGRREEYDVLDKRRGRDPEMGGK
PQRRKNPQEGLYNELQKDKMAEAYSEIGMKGERRRGKGHDGLYQGLSTATKDTYDAL
HMQALPPR

What is claimed is:

1. A biparatopic fusion protein comprising (a) a first antigen-binding protein, or fragment, that binds a tumor antigen; (b) a second antigen-binding protein, or fragment, that binds the tumor antigen; and (c) a polypeptide antigen that is a target for a cellular therapeutic, antibody, or antibody-drug conjugate.

2. The biparatopic fusion protein of claim 1, wherein the tumor antigen is a tumor specific antigen (TSA).

3. The biparatopic fusion protein of claim 1, wherein the tumor antigen is a tumor associated antigen (TAA).

4. The biparatopic fusion protein of any one of claim 1-3, wherein the first antigen-binding protein or fragment is an scFv.

5. The biparatopic fusion protein of any one of claims 1-3, wherein the second antigen-binding protein or fragment is a VHH.

6. The biparatopic fusion protein of any one of claims 1-3, wherein the first antigen-binding protein or fragment and the second antigen-binding protein or fragment are a VHH.

7. The biparatopic fusion protein of any one of claims 1-3, wherein the first antigen-binding protein or fragment and the second antigen-binding protein or fragment are a scFv, an affibody, an adnectin, or an ankyrin repeat protein.

8. The biparatopic fusion protein of any one of claims 1-7, wherein the tumor antigen is CLL-1.

9. The biparatopic fusion protein of any one of claims 1-8, wherein the polypeptide antigen is a CD19 variant comprising at least one amino acid substitution of the amino acid sequence of SEQ ID NO:2.

10. The biparatopic fusion protein of any one of claims 1-9, wherein the CD19 variant comprises one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6.

11. The biparatopic fusion protein of any one of claims 5-10, wherein the VHH comprises the amino acid sequence of any one of SEQ ID Nos:203-225, or a fragment thereof.

12. The biparatopic fusion protein of claim 11, wherein the VHH comprises a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

13. The biparatopic fusion protein of any one of claims 5-7, wherein the VHH comprises CDR1, CDR2, and CDR3 of Group 1; CDR1, CDR2, and CDR3 of Group 2; CDR1, CDR2, and CDR3 of Group 3; CDR1, CDR2, and CDR3 of Group 4; CDR1, CDR2, and CDR3 of Group 5; CDR1, CDR2, and CDR3 of Group 6; CDR1, CDR2, and CDR3 of Group 7; CDR1, CDR2, and CDR3 of Group 8; CDR1, CDR2, and CDR3 of Group 9; CDR1, CDR2, and CDR3 of Group 10; or CDR1, CDR2, and CDR3 of Group 13, depicted in Table 5A and/or Table 5B.

14. The biparatopic fusion protein of any one of claims 1-13, wherein the cellular therapeutic is a CAR-T cell, CAR-NK cell, TCR-T cell, TIL cell, allogenic NK cell, or autologous NK cell.

15. The biparatopic fusion protein of claim 14, wherein the CAR-T or CAR-NK is allogenic.

16. The biparatopic fusion protein of claim 14, wherein the CAR-T or CAR-NK is autologous.

17. A cell comprising:
(i) an antigen binding receptor comprising an antigen-binding domain that binds a first tumor antigen, a transmembrane domain, and a cytosolic signaling domain; and
(ii) a constitutive expression construct encoding a biparatopic fusion protein comprising (a) a first antigen-binding protein, or fragment, that binds a second tumor antigen; (b) a second antigen-binding protein, or fragment, that binds the second tumor antigen; and (c) a polypeptide antigen that is a target for a cellular therapeutic, antibody, or antibody-drug conjugate.

18. The cell of claim 17, wherein the first tumor antigen is CD19.

19. The cell of claim 17 or 18, wherein the tumor antigen is a tumor specific antigen (TSA) or a tumor associated antigen (TAA).

20. The cell of claim 17 or 18, wherein the tumor antigen is a tumor associated antigen (TAA).

21. The cell of any one of claims 17-20, wherein the first antigen-binding protein or fragment is an scFv.

22. The cell of any one of claims 17-20, wherein the second antigen-binding protein or fragment is a VHH.

23. The cell of any one of claims 17-20, wherein the first antigen-binding protein or fragment and the second antigen-binding protein or fragment are a VHH.

24. The cell of any one of claims 17-20, wherein the first antigen-binding protein or fragment and the second antigen-binding protein or fragment are a scFv, an Fab, an affibody, an adnectin, or an ankyrin repeat protein.

25. The cell of any one of claims 17-24, wherein the second tumor antigen is CLL-1.

26. The cell of any one of claims 17-25, wherein the polypeptide antigen is a CD19 variant comprising at least one amino acid substitution of the amino acid sequence of SEQ ID NO:2.

27. The cell of claim 26, wherein the CD19 variant comprises one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6.

28. The T-cell of any one of claims 22-27, wherein the VHH comprises the amino acid sequence of any one of SEQ ID Nos:203-225, or a fragment thereof.

29. The cell of any one of claims 22-27, wherein the VHH comprises a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

30. The cell of any one of claims 22-27, wherein the VHH comprises CDR1, CDR2, and CDR3 of Group 1; CDR1, CDR2, and CDR3 of Group 2; CDR1, CDR2, and CDR3 of Group 3; CDR1, CDR2, and CDR3 of Group 4; CDR1, CDR2, and CDR3 of Group 5; CDR1, CDR2, and CDR3 of Group 6; CDR1, CDR2, and CDR3 of Group 7; CDR1, CDR2, and CDR3 of Group 8; CDR1, CDR2, and CDR3 of Group 9; CDR1, CDR2, and CDR3 of Group 10; or CDR1, CDR2, and CDR3 of Group 13, depicted in Table 5A and/or Table 5B.

31. The cell of any one of claims 17-30, wherein the cellular therapeutic is a CAR-T cell, CAR-NK cell, TCR-T cell, TIL cell, allogenic NK cell, or autologous NK cell.

32. The cell of claim 31, wherein the CAR-T or CAR-NK is allogenic.

33. The cell of claim 31, wherein the CAR-T or CAR-NK is autologous.

34. A cell comprising:
(i) an antigen binding receptor comprising an antigen-binding domain that binds a first tumor antigen, a transmembrane domain, and a cytosolic signaling domain; and
(ii) an inducible expression construct encoding a biparatopic fusion protein comprising (a) a first antigen-binding protein, or fragment, that binds a second tumor antigen; (b) a second antigen-binding protein, or fragment, that binds the second tumor antigen; and (c) a polypeptide antigen that is a target for a cellular therapeutic, antibody, or antibody-drug conjugate.

35. The cell of claim 34, wherein the first tumor antigen is CD19.

36. The cell of claim 34 or 35, wherein the second tumor antigen is a tumor specific antigen (TSA) or a tumor associated antigen (TAA).

37. The cell of claim 34 or 35, wherein the second tumor antigen is a tumor associated antigen (TAA).

38. The cell of any one of claims 34-37, wherein the first antigen-binding protein or fragment is an scFv.

39. The cell of any one of claims 34-38, wherein the second antigen-binding protein or fragment is a VHH.

40. The cell of any one of claims 34-37, wherein the first antigen-binding protein or fragment and the second antigen-binding protein or fragment are a VHH.

41. The cell of any one of claims 34-37, wherein the first antigen-binding protein or fragment and the second antigen-binding protein or fragment are a scFv, an Fab, an affibody, an adnectin, or an ankyrin repeat protein.

42. The cell of any one of claims 34-41, wherein the second tumor antigen is CLL-1.

43. The cell of any one of claims 34-42, wherein the polypeptide antigen is a CD19 variant comprising at least one amino acid substitution of the amino acid sequence of SEQ ID NO:2.

44. The cell of claim 43, wherein the CD19 variant comprises one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6.

45. The cell of any one of claims 39-44, wherein the VHH comprises the amino acid sequence of any one of SEQ ID Nos:203-225, or a fragment thereof.

46. The cell of any one of claims 39-44, wherein the VHH comprises a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

47. The cell of any one of claims 39-46, wherein the VHH comprises CDR1, CDR2, and CDR3 of Group 1; CDR1, CDR2, and CDR3 of Group 2; CDR1, CDR2, and CDR3 of Group 3; CDR1, CDR2, and CDR3 of Group 4; CDR1, CDR2, and CDR3 of Group 5; CDR1, CDR2, and CDR3 of Group 6; CDR1, CDR2, and CDR3 of Group 7; CDR1, CDR2, and CDR3 of Group 8; CDR1, CDR2, and CDR3 of Group 9; CDR1, CDR2, and CDR3 of Group 10; or CDR1, CDR2, and CDR3 of Group 13, depicted in Table 5A and/or Table 5B.

48. The cell of any one of claims 34-47, wherein the cellular therapeutic is an autologous CAR-T cell, an allogenic CAR-T cell, an allogenic CAR-NK cell, TCR-T cell, TIL cell, allogenic NK cell, autologous NK cell, a gamma delta T cell, or a IPSC derived cell therapeutic cell.

49. The cell of any one of claims 34-48, wherein the inducible expression construct comprises a promoter operably linked to a nucleotide encoding the biparatopic fusion protein.

50. The cell of claim 49, wherein the promoter is an IL-2 promoter, a cell surface protein promoter (e.g., CD69 promoter), a cytokine promoter (e.g., TNF promoter), a cellular activation promoter (e.g., CTLA4, OX40, CD40L), or a cell surface adhesion protein promoter (e.g., VLA-1 promoter).

51. A vector comprising a nucleotide sequence encoding the biparatopic fusion protein of any one of claims 1-16.

52. The vector of claim 51, wherein the vector is a viral vector (e.g., an AAV, AAVP, or oncolytic vector).

53. A method of treating a subject having a tumor, comprising administering to the subject the biparatopic fusion protein of any one of claims 1-16 or the vector of claim 51 or 52.

54. The method of claim 53, further comprising administering an antibody, an antibody drug conjugate, or a CAR-T cell to the subject, wherein the antibody, the antibody drug conjugate, or the CAR-T cell targets the polypeptide antigen.

55. The method of claim 54, wherein upon administration, the antibody, antibody drug conjugate, or CAR-T cell binds to the biparatopic fusion protein comprising the polypeptide antigen.

56. The method of claim 55, wherein binding of the antibody, antibody drug conjugate, or CAR-T cell to the biparatopic fusion protein comprising the polypeptide antigen induces killing of the tumor.

57. The method of claim 54, wherein the CAR-T cell is allogenic.

58. The method of claim 54, wherein the CAR-T cell is autologous.

59. A method of treating a subject having a tumor, comprising administering to the subject a T-cell of any one of claims 17-50.

60. A biparatopic fusion protein comprising:
(a) a CD19 variant comprising one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6;
(b) a VHH comprising the amino acid sequence of any one of SEQ ID Nos:203-225, or a fragment thereof, and
(c) an scFv comprising amino acids 20 to 265 of SEQ ID NO. 307.

61. A biparatopic fusion protein comprising:
(a) a CD19 variant comprising one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6;
(b) a VHH comprising the amino acid sequence of any one of SEQ ID Nos:203-225, or a fragment thereof, and
(c) a VHH comprising the amino acid sequence of any one of SEQ ID Nos:203-225, or a fragment thereof.

62. A biparatopic fusion protein comprising SEQ ID NO. 307.

63. A biparatopic fusion protein comprising SEQ ID NO. 311.

64. A biparatopic fusion protein comprising SEQ ID NO. 323.

65. A biparatopic fusion protein comprising SEQ ID NO. 327.

66. A fusion protein comprising SEQ ID NO. 321.

67. A biparatopic fusion protein comprising (a) a first antigen-binding protein, or fragment, that binds a first tumor antigen; (b) a second antigen-binding protein, or fragment, that binds the first tumor antigen; (c) a third antigen-binding protein, or fragment, that binds a second tumor antigen; and (d) a polypeptide antigen that is a target for a cellular therapeutic, antibody, or antibody-drug conjugate.

68. The biparatopic fusion protein of claim 67, wherein the first and second tumor antigens are tumor specific antigens (TSA).

69. The biparatopic fusion protein of claim 67, wherein the first and second tumor antigens are tumor associated antigens (TAA).

70. The biparatopic fusion protein of any one of claim 67-69, wherein the first antigen-binding protein or fragment is an scFv.

71. The biparatopic fusion protein of any one of claims 67-69, wherein the second antigen-binding protein or fragment is a VHH.

72. The biparatopic fusion protein of any one of claims 67-69, wherein the third antigen-binding protein or fragment is a VHH.

73. The biparatopic fusion protein of any one of claims 67-69, wherein the second antigen-binding protein or fragment is an scFv.

74. The biparatopic fusion protein of any one of claims 67-69, wherein the first antigen-binding protein or fragment and the second antigen-binding protein or fragment are a VHH.

75. The biparatopic fusion protein of any one of claims 67-69 wherein the first antigen-binding protein or fragment and the second antigen-binding protein or fragment are a scFv, an affibody, an adnectin, or an ankyrin repeat protein.

76. The biparatopic fusion protein of any one of claims 67-75, wherein the first tumor antigen is CLL-1.

77. The biparatopic fusion protein of any one of claims 67-76, wherein the second tumor antigen is CD33.

78. The biparatopic fusion protein of any one of claims 67-76, wherein the second tumor antigen is IL1RAP.

79. The biparatopic fusion protein of any one of claims 67-78, wherein the polypeptide antigen is a CD19 variant comprising at least one amino acid substitution of the amino acid sequence of SEQ ID NO:2.

80. The biparatopic fusion protein of any one of claims 67-79, wherein the CD19 variant comprises one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6.

81. The biparatopic fusion protein of any one of claims 70-80, wherein the VHH comprises the amino acid sequence of any one of SEQ ID Nos:203-225, or a fragment thereof.

82. The biparatopic fusion protein of claim 81, wherein the VHH comprises a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

83. The biparatopic fusion protein of any one of claims 71-75, wherein the VHH comprises CDR1, CDR2, and CDR3 of Group 1; CDR1, CDR2, and CDR3 of Group 2; CDR1, CDR2, and CDR3 of Group 3; CDR1, CDR2, and CDR3 of Group 4; CDR1, CDR2, and CDR3 of Group 5; CDR1, CDR2, and CDR3 of Group 6; CDR1, CDR2, and CDR3 of Group 7; CDR1, CDR2, and CDR3 of Group 8; CDR1, CDR2, and CDR3 of Group 9; CDR1, CDR2, and CDR3 of Group 10; or CDR1, CDR2, and CDR3 of Group 13, depicted in Table 5A and/or Table 5B.

84. The biparatopic fusion protein of any one of claims 67-83, wherein the cellular therapeutic is a CAR-T cell, CAR-NK cell, TCR-T cell, TIL cell, allogenic NK cell, or autologous NK cell.

85. The biparatopic fusion protein of claim 84, wherein the CAR-T or CAR-NK is allogenic.

86. The biparatopic fusion protein of claim 84, wherein the CAR-T or CAR-NK is autologous.

87. A fusion protein comprising (a) two or more of the same antigen-binding protein(s), or fragment(s), that each bind the same epitope of a tumor antigen; and (b) a polypeptide antigen that is a target for a cellular therapeutic, antibody, or antibody-drug conjugate.

88. The fusion protein of claim 87, wherein the antigen-binding protein, or fragment, is an scFv, VHH, affibody, adnectin, or ankyrin repeat protein.

89. The fusion protein of claim 87, wherein the tumor antigen is a tumor specific antigen (TSA).

90. The fusion protein of claim 87, wherein the tumor antigen is a tumor associated antigen (TAA).

91. The fusion protein of claim 88, wherein the antigen-binding protein, or fragment, is a VHH.

92. The fusion protein of any one of claims 87-91, wherein the tumor antigen is CLL-1.

93. The fusion protein of any one of claims 87-92, wherein the polypeptide antigen is a CD19 variant comprising at least one amino acid substitution of the amino acid sequence of SEQ ID NO:2.

94. The fusion protein of any one of claim 93, wherein the CD19 variant comprises one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6.

95. The fusion protein of any one of claims 91-94, wherein the VHH comprises the amino acid sequence of any one of SEQ ID Nos:203-225, or a fragment thereof.

96. The fusion protein of claim 95, wherein the VHH comprises a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

97. The fusion protein of any one of claims 91-94, wherein the VHH comprises CDR1, CDR2, and CDR3 of Group 1; CDR1, CDR2, and CDR3 of Group 2; CDR1, CDR2, and CDR3 of Group 3; CDR1, CDR2, and CDR3 of Group 4; CDR1, CDR2, and CDR3 of Group 5; CDR1, CDR2, and CDR3 of Group 6; CDR1, CDR2, and CDR3 of Group 7; CDR1, CDR2, and CDR3 of Group 8; CDR1, CDR2, and CDR3 of Group 9; CDR1, CDR2, and CDR3 of Group 10; or CDR1, CDR2, and CDR3 of Group 13, depicted in Table 5A and/or Table 5B.

98. The fusion protein of any one of claims 87-97, wherein the cellular therapeutic is a CAR-T cell, CAR-NK cell, TCR-T cell, TIL cell, allogenic NK cell, or autologous NK cell.

99. The fusion protein of claim 98, wherein the CAR-T or CAR-NK is allogenic.

100. The fusion protein of claim 98, wherein the CAR-T or CAR-NK is autologous.

101. A fusion protein comprising (a) a first antigen-binding protein, or fragment, that binds a tumor antigen; (b) a second antigen-binding protein, or fragment, that binds a second tumor antigen; and (c) a polypeptide antigen that is a target for a cellular therapeutic, antibody, or antibody-drug conjugate.

102. The fusion protein of claim 101, wherein the first or second tumor antigen is a tumor specific antigen (TSA).

103. The fusion protein of claim 1, wherein the first or second tumor antigen is a tumor associated antigen (TAA).

104. The fusion protein of any one of claim 101-103, wherein the first antigen-binding protein or fragment is an scFv.

105. The fusion protein of any one of claims 101-104, wherein the second antigen-binding protein or fragment is a VHH.

106. The fusion protein of any one of claims 101-105, wherein the first tumor antigen is CD33.

107. The fusion protein of any one of claims 101-105, wherein the first tumor antigen is IL1RAP.

108. The fusion protein of any one of claims 101-107, wherein the second tumor antigen is CLL-1.

109. The fusion protein of any one of claims 101-108, wherein the polypeptide antigen is a CD19 variant comprising at least one amino acid substitution of the amino acid sequence of SEQ ID NO:2.

110. The fusion protein of any one of claims 101-109, wherein the CD19 variant comprises one or more amino acid substitutions of SEQ ID NO:2 listed in Table 1A, Table 1B, Table 2A, Table 2B, Table 3, Table 6, FIG. 3, FIG. 4B, FIG. 5A, FIG. 5B, FIG. 5C, FIG. 5D, or FIG. 6.

111. The fusion protein of any one of claims 105-110, wherein the VHH comprises the amino acid sequence of any one of SEQ ID Nos:203-225, or a fragment thereof.

112. The fusion protein of claim 111, wherein the VHH comprises a portion (e.g., a CLL-1 binding portion) of the amino acid sequence of any one of SEQ ID Nos:203-225, wherein the portion lacks all of the C-terminal amino acids TSGPGGQGAEQKLISEEDLGAHHHHHHGAS depicted in each of SEQ ID Nos:203-225.

113. The fusion protein of any one of claims 105-107, wherein the VHH comprises CDR1, CDR2, and CDR3 of Group 1; CDR1, CDR2, and CDR3 of Group 2; CDR1, CDR2, and CDR3 of Group 3; CDR1, CDR2, and CDR3 of Group 4; CDR1, CDR2, and CDR3 of Group 5; CDR1, CDR2, and CDR3 of Group 6; CDR1, CDR2, and CDR3 of Group 7; CDR1, CDR2, and CDR3 of Group 8; CDR1, CDR2, and CDR3 of Group 9; CDR1, CDR2, and CDR3 of Group 10; or CDR1, CDR2, and CDR3 of Group 13, depicted in Table 5A and/or Table 5B.

114. The fusion protein of any one of claims 101-113, wherein the cellular therapeutic is a CAR-T cell, CAR-NK cell, TCR-T cell, TIL cell, allogenic NK cell, or autologous NK cell.

115. The fusion protein of claim 114, wherein the CAR-T or CAR-NK is allogenic.

116. The fusion protein of claim 114, wherein the CAR-T or CAR-NK is autologous.


SEQUENCE LISTING (390 sequences)

SEQ ID NO: 1 - DNA, 816 bp, Homo sapiens
cccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaag

SEQ ID NO: 2 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 3-202 - skipped (200 entries)

SEQ ID NO: 203 - protein, 148 aa, Artificial Sequence
  Description of Artificial Sequence: Synthetic polypeptide
QVQLQESGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHHGAS

SEQ ID NO: 204 - protein, 148 aa, Artificial Sequence
  Description of Artificial Sequence: Synthetic polypeptide
QVQLQESGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTLVTVSSTSGPGGQGAEQKLISEEDLGAHHHHHHGAS

SEQ ID NO: 205 - protein, 148 aa, Artificial Sequence
  Description of Artificial Sequence: Synthetic polypeptide
QVQLQQSGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHHGAS

SEQ ID NO: 206 - protein, 148 aa, Artificial Sequence
  Description of Artificial Sequence: Synthetic polypeptide
QVQLQQSGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTLVTVSSTSGPGGQGAEQKLISEEDLGAHHHHHHGAS

SEQ ID NO: 207 - protein, 148 aa, Artificial Sequence
  Description of Artificial Sequence: Synthetic polypeptide
QVQLQQFGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTLVTVSSTSGPGGQGAEQKLISEEDLGAHHHHHHGAS

SEQ ID NO: 208 - protein, 148 aa, Artificial Sequence
  Description of Artificial Sequence: Synthetic polypeptide
QVQLQASGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHHGAS

SEQ ID NO: 209 - protein, 148 aa, Artificial Sequence
  Description of Artificial Sequence: Synthetic polypeptide
QVQLQEFGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTLVTVSSTSGPGGQGAEQKLISEEDLGAHHHHHHGAS

SEQ ID NO: 210 - protein, 148 aa, Artificial Sequence
  Description of Artificial Sequence: Synthetic polypeptide
QVQLQEFGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHHGAS

SEQ ID NO: 211 - protein, 148 aa, Artificial Sequence
  Description of Artificial Sequence: Synthetic polypeptide
QVQLVESGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTLVTVSSTSGPGGQGAEQKLISEEDLGAHHHHHHGAS

SEQ ID NO: 212 - protein, 148 aa, Artificial Sequence
  Description of Artificial Sequence: Synthetic polypeptide
QVQLQQSGGGLAQTGGSLILSCAASGSIFAINEINLMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHHGAS

SEQ ID NO: 213 - protein, 148 aa, Artificial Sequence
  Description of Artificial Sequence: Synthetic polypeptide
QVQLQESGGGLVQPGGSLRLSCAASGSIFAINEINLMGWYRQAPGKQRELVAACASDGNTYYADSVKGRFTISRDNAEKTVYLQMNNLKPDDTAVYYCDANSRGNYYSGQGTLVTVSSTSGPGGQGAEQKLISEEDLGAHHHHHHGAS

SEQ ID NO: 214 - protein, 145 aa, Artificial Sequence
  Description of Artificial Sequence: Synthetic polypeptide
QVQLQESGGGLVQVGESLRLSCVVSGDTRSINLMGWYRQAPGKQRELVAACASDGNTYYADSVKGRFTISRDNAEKTVYLQMNNLKPDDTAVYYCDANSRGNYYSGQGTLVTVSSTSGPGGQGAEQKLISEEDLGAHHHHHHGAS

SEQ ID NO: 215 - protein, 145 aa, Artificial Sequence
  Description of Artificial Sequence: Synthetic polypeptide
QVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHHGAS

SEQ ID NO: 216 - protein, 156 aa, Artificial Sequence
  Description of Artificial Sequence: Synthetic polypeptide
QVQLQESGGGLVQPGGSLRLSCAASGFTFNSYAMTWVRQAPGKGLEWVSDINSGGGSTNYADSVKGRFTISRDNAKNTLYLQMNSLKPEDTAVYYCATELRGSDYYRGPIREYAYWGQGTLVTVSSTSGPGGQGAEQKLISEEDLGAHHHHHHGAS

SEQ ID NO: 217 - protein, 156 aa, Artificial Sequence
  Description of Artificial Sequence: Synthetic polypeptide
QVQLQESGGALVQAGGSLRLSCAASGLTFSNYAMGWFRQAPGKEREFVAAINWSGGTTDYATSVKGRFTISRDNAKNTVYLQLNSLKPEDTAVYYCAASYRLRITVVVTPDEYHYWGQGTLVTVSSTSGPGGQGAEQKLISEEDLGAHHHHHHGAS

SEQ ID NO: 218 - protein, 151 aa, Artificial Sequence
  Description of Artificial Sequence: Synthetic polypeptide
QVQLQQSGGGLVQPGGSLRLSCAASGFAFDDYAMIWVRQGPGKGLEWVSSISWNGGGTYYAESIVGRFTVSRDNAKKMVYLQMNGLKSEDTAMYYCVKLVDSGWYSAYDYWGQGTQVTVSSTSGPGGQGAEQKLISEEDLGAHHHHHHGAS

SEQ ID NO: 219 - protein, 150 aa, Artificial Sequence
  Description of Artificial Sequence: Synthetic polypeptide
QVQLQESGGGLVQAGGSLRLSCVVSGATSNVNAMGWYRQAPGKERELVAAISSGGSTSYRDSVKGRFTISRDNAKNTLYLQMNSLKPEDTAMYYCAAQDWATEGYEYDYWGQGTLVTVSSTSGPGGQGAEQKLISEEDLGAHHHHHHGAS

SEQ ID NO: 220 - protein, 144 aa, Artificial Sequence
  Description of Artificial Sequence: Synthetic polypeptide
QVQLQAFGGGLVQPGGSLRLSCVVSGTMFSGKDVNWLRQAPGKHVEVVATVSSDGGTDYADFVKGRFTISRDDAKNTVNLQMNSLEPEDTANYMCHFLWGRHYWGQGTQVTVSSTSGPGGQGAEQKLISEEDLGAHHHHHHGAS

SEQ ID NO: 221 - protein, 156 aa, Artificial Sequence
  Description of Artificial Sequence: Synthetic polypeptide
QVQLQAFGGGMVQAGESLRLSCVASGNDISGSAMAWYRAHLGAERELVAVDAPRERPFYIDPVIGRFTISRDDRNKMLYLQMNDLRPDDTATYWCGPSLRTFHGREWYRPPWFTSWGQGTQVTVSSTSGPGGQGAEQKLISEEDLGAHHHHHHGAS

SEQ ID NO: 222 - protein, 150 aa, Artificial Sequence
  Description of Artificial Sequence: Synthetic polypeptide
QVQLQQSGGGLVQAGGSLRLSCAASGSIFSINAMGWYRQAPGKRREMVAVVSRFGETTYTGSVKGRFTISRINRNNTVFLQMNRLKPEDTAVYYCNARIRGNYGSRIDYWGQGTQVTVSSTSGPGGQGAEQKLISEEDLGAHHHHHHGAS

SEQ ID NO: 223 - protein, 137 aa, Artificial Sequence
  Description of Artificial Sequence: Synthetic polypeptide
QVQLQEFGGGLVQLGGSARLSCVVSGNMLDLNTMAWYRQGELVAALGISTYYAESVKGRFTISRDNAKNTLYLQMNSLKSEDTAVYYCARDYNFESWGQGTLVTVSSTSGPGGQGAEQKLISEEDLGAHHHHHHGAS

SEQ ID NO: 224 - protein, 146 aa, Artificial Sequence
  Description of Artificial Sequence: Synthetic polypeptide
AVQLQESGGGLVQAGGSLRLSCAASGSDRSINVMNWYRQAPGKQRELVAAITSGGTTNYAQSVKGRVTISRDSAKNTVYLQMNSLKPEDTAVYFCKADTRWGGMYWGPGTQVTVSSTSGPGGQGAEQKLISEEDLGAHHHHHHGAS

SEQ ID NO: 225 - protein, 154 aa, Artificial Sequence
  Description of Artificial Sequence: Synthetic polypeptide
QVQLQQSGGGLVQAGGSLTLSCAATGRTIDNGAMAWFRQAPGKQRELVAAINWSGGATFYTDSVKYRFTISRDNVRHTLDLQMTSLKPEDTTIYFCASRRGVDLRRNSYEYDYWGRGTLVTVSSTSGPGGQGAEQKLISEEDLGAHHHHHHGAS

SEQ ID NO: 226-299 - skipped (74 entries)

SEQ ID NO: 300 - DNA, 1647 bp, Artificial Sequence
  Description of Artificial Sequence: Synthetic polynucleotide
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccaggaggaggtgggtctggaggtggaggatctggtggaggtgggtctggaggaggtgggtctgacatggcccaggtgcagctgcaggagagcggccccggcctggtgaagcccagcgagaccctgagcctgacctgcgtggtgagcggcggcagcatcagcagcagcaactggtggagctgggtgaggcagccccccggcaagggcctggagtggatcggcgagatctaccacagcggcagccccgactacaaccccagcctgaagagcagggtgaccatcagcgtggacaagagcaggaaccagttcagcctgaagctgagcagcgtgaccgccgccgacaccgccgtgtactactgcgccaaggtgagcaccggcggcttcttcgactactggggccagggcaccctggtgaccgtgagcagcggcggcggcggcagcggcggcggcggcagcggcggcggcggcagcgagatcgagctgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcaccccccccaccttcggccccggcaccaaggtggagatcaagaggacccaccaccaccaccaccac

SEQ ID NO: 301 - protein, 549 aa, Artificial Sequence
  Description of Artificial Sequence: Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPGGGGSGGGGSGGGGSGGGGSDMAQVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGPGTKVEIKRTHHHHHH

SEQ ID NO: 302 - DNA, 1377 bp, Artificial Sequence
  Description of Artificial Sequence: Synthetic polynucleotide
atgccacctcctcgcctcctcttcttcctcctcttcctcacccccatggaagtcaggcccgaggagcccctggtggtgaaggtggaggagggcgacaccgccgtgctgccctgcctgaagggcaccagcgacggccccacccagcagctgacctggagcagggagagccccctgaagcccttcctgaagtacagcctgggcgtgcccggcctgggcgtgcacgtgaggcccgacgccatcagcgtggtgatcaggaacgtgagccagcagatgggcggcttctacctgtgccagcccggcccccccagcgagaaggcctggcagcccggctggaccgtgaacgtggagggcagcggcgagctgttcaggtggaacgtgagcgacctgggcggcctgggctgcggcctgaagaacaggagcagcgagggccccagcagccccagcggcaagctgatgagccccaagctgtacgtgtgggccaaggacaggcccgagatctgggagggcgagcccccctgcctgccccccagggacagcctgaaccagagcctgagccaggacctgaccatggcccccggcagcaccctgtggctgagctgcggcgtgccccccgacagcgtgagcaggggccccctgagctggacccacgtgcaccccaagggccccaagagcctgctgagcctggagctgaaggacgacaggcccgccagggagatgatcgtggacgagaccggcctgctgctgcccagggccaccgcccaggacgccggcaagtggtactgcagcaggggcaacgtgaccaccagctaccacctggagatcaccgccaggcccgtgaaggcccacagcgacctgaggaccggcggctggaagggaggaggtgggtctggaggtggaggatctggtggaggtgggtctggaggaggtggatcccaggtgcagctgcaagcgtctgggggaggcttggtgcaggctggggggtctctgagactctcctgtgcagcctcaggaagcatcttcgctattaatgaaatcaatcttatggggtggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggcgcagaacaaaaactcatctcagaagaggatctgggcgcacaccatcaccaccatcatggcgcatct

SEQ ID NO: 303 - protein, 459 aa, Artificial Sequence
  Description of Artificial Sequence: Synthetic polypeptide
MPPPRLLFFLLFLTPMEVRPEEPLVVKVEEGDTAVLPCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPAREMIVDETGLLLPRATAQDAGKWYCSRGNVTTSYHLEITARPVKAHSDLRTGGWKGGGGSGGGGSGGGGSGGGGSQVQLQASGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHHGAS

SEQ ID NO: 304 - DNA, 1290 bp, Artificial Sequence
  Description of Artificial Sequence: Synthetic polynucleotide
atggagtttgggctgagctgggttttcctcgttgctctttttagaggtgtccagtgtcaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggcgcaggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagacatcatcaccatcaccat

SEQ ID NO: 305 - protein, 430 aa, Artificial Sequence
  Description of Artificial Sequence: Synthetic polypeptide
MEFGLSWVFLVALFRGVQCQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSTSGPGGQGAGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRHHHHHH

SEQ ID NO: 306 - DNA, 2085 bp, Artificial Sequence
  Description of Artificial Sequence: Synthetic polynucleotide
atggagtttgggctgagctgggttttcctcgttgctctttttagaggtgtccagtgtgacatggcccaggtgcagctgcaggagagcggccccggcctggtgaagcccagcgagaccctgagcctgacctgcgtggtgagcggcggcagcatcagcagcagcaactggtggagctgggtgaggcagccccccggcaagggcctggagtggatcggcgagatctaccacagcggcagccccgactacaaccccagcctgaagagcagggtgaccatcagcgtggacaagagcaggaaccagttcagcctgaagctgagcagcgtgaccgccgccgacaccgccgtgtactactgcgccaaggtgagcaccggcggcttcttcgactactggggccagggcaccctggtgaccgtgagcagcggcggcggcggcagcggcggcggcggcagcggcggcggcggcagcgagatcgagctgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcaccccccccaccttcggccccggcaccaaggtggagatcaagaggaccggcggcggaggatctggcggaggtggaagcggaggcggaggaagcggtggcggcggatctcaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggtgcaggaggaggggggtctgggggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagacatcatcaccatcaccat

SEQ ID NO: 307 - protein, 695 aa, Artificial Sequence
  Description of Artificial Sequence: Synthetic polypeptide
MEFGLSWVFLVALFRGVQCDMAQVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGPGTKVEIKRTGGGGSGGGGSGGGGSGGGGSQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSTSGPGGQGAGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRHHHHHH

SEQ ID NO: 308 - DNA, 3333 bp, Artificial Sequence
  Description of Artificial Sequence: Synthetic polynucleotide
atgaggcttctggtgcttctttggggttgcttgctgttgcccggttacgaagcagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacaaggtaatacgcttccgtacacgttcggaggggggactaagttggaaataacaggaggcggcgggtctggaggtggaggatctggtggtggcgggtctggaggcggcgggtctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactacaactcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgccgcgcgaaacgcagcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtcccgcggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggcctggcggcggcgggtctggaggtggaggatctggtggtggcgggtctggtggcggcgggtctgacatggcccaggtgcagctgcaggagagcggccccggcctggtgaagcccagcgagaccctgagcctgacctgcgtggtgagcggcggcagcatcagcagcagcaactggtggagctgggtgaggcagccccccggcaagggcctggagtggatcggcgagatctaccacagcggcagccccgactacaaccccagcctgaagagcagggtgaccatcagcgtggacaagagcaggaaccagttcagcctgaagctgagcagcgtgaccgccgccgacaccgccgtgtactactgcgccaaggtgagcaccggcggcttcttcgactactggggccagggcaccctggtgaccgtgagcagcggcggcggcggcagcggcggcggcggcagcggcggcggcggcagcgagatcgagctgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcaccccccccaccttcggccccggcaccaaggtggagatcaagaggacccaccaccaccaccaccac

SEQ ID NO: 309 - protein, 1111 aa, Artificial Sequence
  Description of Artificial Sequence: Synthetic polypeptide
MRLLVLLWGCLLLPGYEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPGGGGSGGGGSGGGGSGGGGSDMAQVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGPGTKVEIKRTHHHHHH

SEQ ID NO: 310 - DNA, 3771 bp, Artificial Sequence
  Description of Artificial Sequence: Synthetic polynucleotide
atgaggcttctggtgcttctttggggttgcttgctgttgcccggttacgaagcagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacaaggtaatacgcttccgtacacgttcggaggggggactaagttggaaataacaggaggcggcgggtctggaggtggaggatctggtggtggcgggtctggaggcggcgggtctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactacaactcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgccgcgcgaaacgcagcggcagcggcgcgaccaactttagcctgctgaaacaggcgggcgatgtggaagaaaacccgggcccgatggagtttgggctgagctgggttttcctcgttgctctttttagaggtgtccagtgtgacatggcccaggtgcagctgcaggagagcggccccggcctggtgaagcccagcgagaccctgagcctgacctgcgtggtgagcggcggcagcatcagcagcagcaactggtggagctgggtgaggcagccccccggcaagggcctggagtggatcggcgagatctaccacagcggcagccccgactacaaccccagcctgaagagcagggtgaccatcagcgtggacaagagcaggaaccagttcagcctgaagctgagcagcgtgaccgccgccgacaccgccgtgtactactgcgccaaggtgagcaccggcggcttcttcgactactggggccagggcaccctggtgaccgtgagcagcggcggcggcggcagcggcggcggcggcagcggcggcggcggcagcgagatcgagctgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcaccccccccaccttcggccccggcaccaaggtggagatcaagaggaccggcggcggaggatctggcggaggtggaagcggaggcggaggaagcggtggcggcggatctcaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggtgcaggaggaggggggtctgggggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagacatcatcaccatcaccat

SEQ ID NO: 311 - protein, 1257 aa, Artificial Sequence
  Description of Artificial Sequence: Synthetic polypeptide
MRLLVLLWGCLLLPGYEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMEFGLSWVFLVALFRGVQCDMAQVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGPGTKVEIKRTGGGGSGGGGSGGGGSGGGGSQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSTSGPGGQGAGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRHHHHHH

SEQ ID NO: 312 - DNA, 3054 bp, Artificial Sequence
  Description of Artificial Sequence: Synthetic polynucleotide
atgaggcttctggtgcttctttggggttgcttgctgttgcccggttacgaagcagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacaaggtaatacgcttccgtacacgttcggaggggggactaagttggaaataacaggaggcggcgggtctggaggtggaggatctggtggtggcgggtctggaggcggcgggtctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactacaactcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgccgcgcgaaacgcagcggcagcggcgcgaccaactttagcctgctgaaacaggcgggcgatgtggaagaaaacccgggcccgatgccacctcctcgcctcctcttcttcctcctcttcctcacccccatggaagtcaggcccgaggagcccctggtggtgaaggtggaggagggcgacaccgccgtgctgccctgcctgaagggcaccagcgacggccccacccagcagctgacctggagcagggagagccccctgaagcccttcctgaagtacagcctgggcgtgcccggcctgggcgtgcacgtgaggcccgacgccatcagcgtggtgatcaggaacgtgagccagcagatgggcggcttctacctgtgccagcccggcccccccagcgagaaggcctggcagcccggctggaccgtgaacgtggagggcagcggcgagctgttcaggtggaacgtgagcgacctgggcggcctgggctgcggcctgaagaacaggagcagcgagggccccagcagccccagcggcaagctgatgagccccaagctgtacgtgtgggccaaggacaggcccgagatctgggagggcgagcccccctgcctgccccccagggacagcctgaaccagagcctgagccaggacctgaccatggcccccggcagcaccctgtggctgagctgcggcgtgccccccgacagcgtgagcaggggccccctgagctggacccacgtgcaccccaagggccccaagagcctgctgagcctggagctgaaggacgacaggcccgccagggagatgatcgtggacgagaccggcctgctgctgcccagggccaccgcccaggacgccggcaagtggtactgcagcaggggcaacgtgaccaccagctaccacctggagatcaccgccaggcccgtgaaggcccacagcgacctgaggaccggcggctggaagggaggaggtgggtctggaggtggaggatctggtggaggtgggtctggaggaggtggatcccaggtgcagctgcaagcgtctgggggaggcttggtgcaggctggggggtctctgagactctcctgtgcagcctcaggaagcatcttcgctattaatgaaatcaatcttatggggtggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggcgcagaacaaaaactcatctcagaagaggatctgggcgcacaccatcaccaccatcat

SEQ ID NO: 313 - protein, 1018 aa, Artificial Sequence
  Description of Artificial Sequence: Synthetic polypeptide
MRLLVLLWGCLLLPGYEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDTAVLPCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPAREMIVDETGLLLPRATAQDAGKWYCSRGNVTTSYHLEITARPVKAHSDLRTGGWKGGGGSGGGGSGGGGSGGGGSQVQLQASGGGLVQAGGSLRLSCAASGSIFAINEINLMGWYRQAPGKQRELVAACASDGNTYYADSVKGRFTISRDNAEKTVYLQMNNLKPDDTAVYYCDANSRGNYYSGQGTQVTVSSTSGPGGQGAEQKLISEEDLGAHHHHHH

SEQ ID NO: 314 - DNA, 2976 bp, Artificial Sequence
  Description of Artificial Sequence: Synthetic polynucleotide
atgaggcttctggtgcttctttggggttgcttgctgttgcccggttacgaagcagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacaaggtaatacgcttccgtacacgttcggaggggggactaagttggaaataacaggaggcggcgggtctggaggtggaggatctggtggtggcgggtctggaggcggcgggtctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactacaactcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgccgcgcgaaacgcagcggcagcggcgcgaccaactttagcctgctgaaacaggcgggcgatgtggaagaaaacccgggcccgatggagtttgggctgagctgggttttcctcgttgctctttttagaggtgtccagtgtcaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggcgcaggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagacatcatcaccatcaccat

SEQ ID NO: 315 - protein, 992 aa, Artificial Sequence
  Description of Artificial Sequence: Synthetic polypeptide
MRLLVLLWGCLLLPGYEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMEFGLSWVFLVALFRGVQCQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSTSGPGGQGAGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRHHHHHH

SEQ ID NO: 316 - DNA, 3345 bp, Artificial Sequence
  Description of Artificial Sequence: Synthetic polynucleotide
atgaggcttctggtgcttctttggggttgcttgctgttgcccggttacgaagcagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacaaggtaatacgcttccgtacacgttcggaggggggactaagttggaaataacaggaggcggcgggtctggaggtggaggatctggtggtggcgggtctggaggcggcgggtctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactacaactcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgccgcgcgaaacgcagcggcagcggcgcgaccaactttagcctgctgaaacaggcgggcgatgtggaagaaaacccgggcccgatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtcccgcggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggcctggcggcggcgggtctggaggtggaggatctggtggtggcgggtctggtggcggcgggtctgaggtgcagctggtggagtctggtggtggtcttgttcaacctggtggttctcttcgtctttcttgtgctgcttctggttttaatattaaagatacttatattcattgggttcgtcaagctcctggtaaaggtcttgaatgggttgctcgtatttatcctactaatggttatactcgttatgctgattctgttaaaggtcgttttactatttctgctgatacttctaaaaatactgcttatcttcaaatgaactctcttcgtgctgaagatactgctgtttattattgttctcgttggggtggtgatggtttttatgctatggattattggggtcaaggtactcttgtcaccgtctcctcagctagcaccgggggcggcgggtctggaggtggaggatctggtggcggcgggtctgacatccagatgacccagtctccttcttctctttctgcttctgttggtgatcgtgttactattacttgtcgtgcttctcaagatgttaatactgctgttgcttggtatcaacaaaaacctggtaaagctcctaaacttcttatttattctgcttcttttctttattctggtgttccttctcgtttttctggttctcgttctggtactgattttactcttactatttcttctcttcaacctgaagattttgctacttattattgtcaacaacattatactactcctcctacttttggtcaaggtaccaaggtggagatcaaacgtacgtctagacatcatcaccatcaccat

SEQ ID NO: 317 - protein, 1115 aa, Artificial Sequence
  Description of Artificial Sequence: Synthetic polypeptide
MRLLVLLWGCLLLPGYEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTSRHHHHHH

SEQ ID NO: 318 - DNA, 1614 bp, Artificial Sequence
  Description of Artificial Sequence: Synthetic polynucleotide
atggagaccgacaccctgctgctgtgggtgctgctgctgtgggtgcccggcagcaccggcgacatggcccaggtgcagctgcaggagagcggccccggcctggtgaagcccagcgagaccctgagcctgacctgcgtggtgagcggcggcagcatcagcagcagcaactggtggagctgggtgaggcagccccccggcaagggcctggagtggatcggcgagatctaccacagcggcagccccgactacaaccccagcctgaagagcagggtgaccatcagcgtggacaagagcaggaaccagttcagcctgaagctgagcagcgtgaccgccgccgacaccgccgtgtactactgcgccaaggtgagcaccggcggcttcttcgactactggggccagggcaccctggtgaccgtgagcagcggcggcggcggcagcggcggcggcggcagcggcggcggcggcagcgagatcgagctgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcaccccccccaccttcggccccggcaccaaggtggagatcaagaggacctcttctgactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 319 - protein, 537 aa, Artificial Sequence
  Description of Artificial Sequence: Synthetic polypeptide
METDTLLLWVLLLWVPGSTGDMAQVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGPGTKVEIKRTSSDYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 320 - DNA, 2100 bp, Artificial Sequence
  Description of Artificial Sequence: Synthetic polynucleotide
atggagtttgggctgagctgggttttcctcgttgctctttttagaggtgtccagtgtcaggtgcagctggtgcagagcggcgccgaggtgaagaagcccggcgagagcgtgaaggtgagctgcaaggccagcggctacaccttcaccaactacggcatgaactgggtgaagcaggcccccggccagggcctggagtggatgggctggatcaacacctacaccggcgagcccacctacgccgacaagttccagggcagggtgaccatgaccaccgacaccagcaccagcaccgcctacatggagatcaggaacctgggcggcgacgacaccgccgtgtactactgcgccaggtggagctggagcgacggctactacgtgtacttcgactactggggccagggcaccagcgtgaccgtgagcagcggcggcggcggcagcggcggcggcggcagcggcggcggcggcagcgacatcgtgatgacccagagccccgacagcctgaccgtgagcctgggcgagaggaccaccatcaactgcaagagcagccagagcgtgctggacagcagcaccaacaagaacagcctggcctggtaccagcagaagcccggccagccccccaagctgctgctgagctgggccagcaccagggagagcggcatccccgacaggttcagcggcagcggcagcggcaccgacttcaccctgaccatcgacagcccccagcccgaggacagcgccacctactactgccagcagagcgcccacttccccatcaccttcggccagggcaccaggctggagatcaagggaggaggtgggtctggaggtggaggatctggtggaggtgggtctggaggaggtggatcccaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggtgcaggaggaggggggtctgggggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagacatcatcaccatcaccat

SEQ ID NO: 321 - protein, 700 aa, Artificial Sequence
  Description of Artificial Sequence: Synthetic polypeptide
MEFGLSWVFLVALFRGVQCQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKGGGGSGGGGSGGGGSGGGGSQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSTSGPGGQGAGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRHHHHHH

SEQ ID NO: 322 - DNA, 2035 bp, Artificial Sequence
  Description of Artificial Sequence: Synthetic polynucleotide
atggagtttgggctgagctgggttttcctcgttgctctttttagaggtgtccagtgtgacatggcccaggtgcagctgcaggagagcggccccggcctggtgaagcccagcgagaccctgagcctgacctgcgtggtgagcggcggcagcatcagcagcagcaactggtggagctgggtgaggcagccccccggcaagggcctggagtggatcggcgagatctaccacagcggcagccccgactacaaccccagcctgaagagcagggtgaccatcagcgtggacaagagcaggaaccagttcagcctgaagctgagcagcgtgaccgccgccgacaccgccgtgtactactgcgccaaggtgagcaccggcggcttcttcgactactggggccagggcaccctggtgaccgtgagcagcggcggcggcggcagcggcggcggcggcagcggcggcggcggcagcgagatcgagctgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcaccccccccaccttcggccccggcaccaaggtggagatcaagaggaccggcggcggaggatctggcggaggtggaagcggaggcggaggaagcggtggcggcggatctcaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaggaggaggggggtctgggggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccat

SEQ ID NO: 323 - protein, 678 aa, Artificial Sequence
  Description of Artificial Sequence: Synthetic polypeptide
MEFGLSWVFLVALFRGVQCDMAQVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGPGTKVEIKRTGGGGSGGGGSGGGGSGGGGSQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARP

SEQ ID NO: 324 - DNA, 1668 bp, Artificial Sequence
  Description of Artificial Sequence: Synthetic polynucleotide
atggagtttgggctgagctgggttttcctcgttgctctttttagaggtgtccagtgtcaggtgcagctggtgcagagcggcgccgaggtgaagaagcccggcgagagcgtgaaggtgagctgcaaggccagcggctacaccttcaccaactacggcatgaactgggtgaagcaggcccccggccagggcctggagtggatgggctggatcaacacctacaccggcgagcccacctacgccgacaagttccagggcagggtgaccatgaccaccgacaccagcaccagcaccgcctacatggagatcaggaacctgggcggcgacgacaccgccgtgtactactgcgccaggtggagctggagcgacggctactacgtgtacttcgactactggggccagggcaccagcgtgaccgtgagcagcggcggcggcggcagcggcggcggcggcagcggcggcggcggcagcgacatcgtgatgacccagagccccgacagcctgaccgtgagcctgggcgagaggaccaccatcaactgcaagagcagccagagcgtgctggacagcagcaccaacaagaacagcctggcctggtaccagcagaagcccggccagccccccaagctgctgctgagctgggccagcaccagggagagcggcatccccgacaggttcagcggcagcggcagcggcaccgacttcaccctgaccatcgacagcccccagcccgaggacagcgccacctactactgccagcagagcgcccacttccccatcaccttcggccagggcaccaggctggagatcaagggaggaggtgggtctggaggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggccatctagacatcatcaccatcaccat

SEQ ID NO: 325 - protein, 556 aa, Artificial Sequence
  Description of Artificial Sequence: Synthetic polypeptide
MEFGLSWVFLVALFRGVQCQVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSSGGGGSGGGGSGGGGSDIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIKGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARPSRHHHHHH

SEQ ID NO: 326 - DNA, 3696 bp, Artificial Sequence
  Description of Artificial Sequence: Synthetic polynucleotide
atgaggcttctggtgcttctttggggttgcttgctgttgcccggttacgaagcagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacaaggtaatacgcttccgtacacgttcggaggggggactaagttggaaataacaggaggcggcgggtctggaggtggaggatctggtggtggcgggtctggaggcggcgggtctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactacaactcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgccgcgcgaaacgcagcggcagcggcgcgaccaactttagcctgctgaaacaggcgggcgatgtggaagaaaacccgggcccgatggagtttgggctgagctgggttttcctcgttgctctttttagaggtgtccagtgtgacatggcccaggtgcagctgcaggagagcggccccggcctggtgaagcccagcgagaccctgagcctgacctgcgtggtgagcggcggcagcatcagcagcagcaactggtggagctgggtgaggcagccccccggcaagggcctggagtggatcggcgagatctaccacagcggcagccccgactacaaccccagcctgaagagcagggtgaccatcagcgtggacaagagcaggaaccagttcagcctgaagctgagcagcgtgaccgccgccgacaccgccgtgtactactgcgccaaggtgagcaccggcggcttcttcgactactggggccagggcaccctggtgaccgtgagcagcggcggcggcggcagcggcggcggcggcagcggcggcggcggcagcgagatcgagctgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcagcctgcagagcggcgtgcccagcaggttcagcggcagcggcagcggcaccgacttcaccctgaccatcagcagcctgcagcccgaggacttcgccacctactactgccagcagagctacagcaccccccccaccttcggccccggcaccaaggtggagatcaagaggaccggcggcggaggatctggcggaggtggaagcggaggcggaggaagcggtggcggcggatctcaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaggaggaggggggtctgggggtggaggatctggtggaggtgggtctggaggaggtggatcccccgaggaacctctagtggtgaaggtggaagagggagataccgctgccctgtggtgcctcaaggggacctcagatggccccactcagcagctgacctggtctcgggagtccccgcttaaacccttcttaaaatacagcctgggggtgccaggcctgggagtgcacgtgaggcccgacgccatcagcgtggttatccggaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccgggacctcaccgttgcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatgggcacgagcctgatgttgccccgggccacagctcaagacgctggaaagtggtattgtcaccgtggcaacctgaccatgtcattccacctggagatcactgctcggcca

SEQ ID NO: 327 - protein, 1232 aa, Artificial Sequence
  Description of Artificial Sequence: Synthetic polypeptide
MRLLVLLWGCLLLPGYEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRRAKRSGSGATNFSLLKQAGDVEENPGPMEFGLSWVFLVALFRGVQCDMAQVQLQESGPGLVKPSETLSLTCVVSGGSISSSNWWSWVRQPPGKGLEWIGEIYHSGSPDYNPSLKSRVTISVDKSRNQFSLKLSSVTAADTAVYYCAKVSTGGFFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGPGTKVEIKRTGGGGSGGGGSGGGGSGGGGSQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSGGGGSGGGGSGGGGSGGGGSPEEPLVVKVEEGDTAALWCLKGTSDGPTQQLTWSRESPLKPFLKYSLGVPGLGVHVRPDAISVVIRNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDLTVAPGSTLWLSCGVPPDSVSRGPLSWTHVHPKGPKSLLSLELKDDRPARDMWVMGTSLMLPRATAQDAGKWYCHRGNLTMSFHLEITARP

SEQ ID NO: 328 - protein, 275 aa, Homo sapiens
MWIDFFTYSSMSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 329 - protein, 275 aa, Homo sapiens [sequence omitted]

SEQ ID NO: 330 - DNA, 1239 bp, Artificial Sequence
  Description of Artificial Sequence: Synthetic polynucleotide
atggagtttgggctgagctgggttttcctcgttgctctttttagaggtgtccagtgtcaggtgcagctgcaggagtctgggggaggcttggtgcaggctggggggtcccttagactctcctgtgtagcctctggaagcatcagaagtatcaatgtcatgggctggtaccgccaggctccagggaagcagcgcgagttggtcgcagcttgtgctagtgatggcaacacatactatgcggactccgtgaagggccgattcaccatctccagagacaacgccgagaaaacggtgtatctgcagatgaacaacctgaaacctgacgacacagccgtctattactgtgatgcgaattcgagggggaattattattcgggccaggggacccaggtcaccgtttcctcaactagtggcccgggaggccaaggcgcagactacaaagacgatgacgacaagattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtccgcagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagccctgccccctcgc

SEQ ID NO: 331 - protein, 413 aa, Artificial Sequence
  Description of Artificial Sequence: Synthetic polypeptide
MEFGLSWVFLVALFRGVQCQVQLQESGGGLVQAGGSLRLSCVASGSIRSINVMGWYRQAPGKQRELVAACASDGNTYYADSVKGRFTISRDNAEKTVYLQMNNLKPDDTAVYYCDANSRGNYYSGQGTQVTVSSTSGPGGQGADYKDDDDKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 332 - protein, 9 aa, Artificial Sequence
  Description of Artificial Sequence: Synthetic peptide
TSGPGGQGA

SEQ ID NO: 333 - protein, 10 aa, Artificial Sequence
  Description of Artificial Sequence: Synthetic peptide
EQKLISEEDL

SEQ ID NO: 334 - protein, 6 aa, Artificial Sequence
  Description of Artificial Sequence: Synthetic 6xHis tag
HHHHHH

SEQ ID NO: 335 - protein, 30 aa, Artificial Sequence
  Description of Artificial Sequence: Synthetic polypeptide
TSGPGGQGAEQKLISEEDLGAHHHHHHGAS

SEQ ID NO: 336 - protein, 13 aa, Artificial Sequence
  Description of Artificial Sequence: Synthetic peptide
CAASGSIFAINEI

SEQ ID NO: 337 - protein, 10 aa, Artificial Sequence
  Description of Artificial Sequence: Synthetic peptide
CVVSGDTRSI

SEQ ID NO: 338 - protein, 10 aa, Artificial Sequence
  Description of Artificial Sequence: Synthetic peptide
CVASGSIRSI

SEQ ID NO: 339 - protein, 12 aa, Artificial Sequence
  Description of Artificial Sequence: Synthetic peptide
CAASGFTFNSYA

SEQ ID NO: 340 - protein, 12 aa, Artificial Sequence
  Description of Artificial Sequence: Synthetic peptide
CAASGLTFSNYA

SEQ ID NO: 341 - protein, 12 aa, Artificial Sequence
  Description of Artificial Sequence: Synthetic peptide
CAASGFAFDDYA

SEQ ID NO: 342 - protein, 12 aa, Artificial Sequence
  Description of Artificial Sequence: Synthetic peptide
CVVSGATSNVNA

SEQ ID NO: 343 - protein, 12 aa, Artificial Sequence
  Description of Artificial Sequence: Synthetic peptide
CVVSGTMFSGKD

SEQ ID NO: 344 - protein, 12 aa, Artificial Sequence
  Description of Artificial Sequence: Synthetic peptide
CVASGNDISGSA

SEQ ID NO: 345 - protein, 12 aa, Artificial Sequence
  Description of Artificial Sequence: Synthetic peptide
CAASGSIFSINA

SEQ ID NO: 346 - protein, 12 aa, Artificial Sequence
  Description of Artificial Sequence: Synthetic peptide
CVVSGNMLDLNT

SEQ ID NO: 347 - protein, 8 aa, Artificial Sequence
  Description of Artificial Sequence: Synthetic peptide
GSDRSINV

SEQ ID NO: 348 - protein, 8 aa, Artificial Sequence
  Description of Artificial Sequence: Synthetic peptide
GRTIDNGA

SEQ ID NO: 349 - protein, 11 aa, Artificial Sequence
  Description of Artificial Sequence: Synthetic peptide
VAACASDGNTY

SEQ ID NO: 350 - protein, 13 aa, Artificial Sequence
  Description of Artificial Sequence: Synthetic peptide
WVSDINSGGGSTN

SEQ ID NO: 351 - protein, 10 aa, Artificial Sequence
  Description of Artificial Sequence: Synthetic peptide
AINWSGGTTD

SEQ ID NO: 352 - protein, 13 aa, Artificial Sequence
  Description of Artificial Sequence: Synthetic peptide
WVSSISWNGGGTY

SEQ ID NO: 353 - protein, 12 aa, Artificial Sequence
  Description of Artificial Sequence: Synthetic peptide
LVAAISSGGSTS

SEQ ID NO: 354 - protein, 11 aa, Artificial Sequence
  Description of Artificial Sequence: Synthetic peptide
VATVSSDGGTD

SEQ ID NO: 355 - protein, 11 aa, Artificial Sequence
  Description of Artificial Sequence: Synthetic peptide
VAVDAPRERPF

SEQ ID NO: 356 - protein, 11 aa, Artificial Sequence
  Description of Artificial Sequence: Synthetic peptide
VAVVSRFGETT

SEQ ID NO: 357 - protein, 10 aa, Artificial Sequence
  Description of Artificial Sequence: Synthetic peptide
LVAALGISTY

SEQ ID NO: 358 - protein, 7 aa, Artificial Sequence
  Description of Artificial Sequence: Synthetic peptide
ITSGGTT

SEQ ID NO: 359 - protein, 8 aa, Artificial Sequence
  Description of Artificial Sequence: Synthetic peptide
INWSGGAT

SEQ ID NO: 360 - protein, 9 aa, Artificial Sequence
  Description of Artificial Sequence: Synthetic peptide
DANSRGNYY

SEQ ID NO: 361 - protein, 19 aa, Artificial Sequence
  Description of Artificial Sequence: Synthetic peptide
ATELRGSDYYRGPIREYAY

SEQ ID NO: 362 - protein, 19 aa, Artificial Sequence
  Description of Artificial Sequence: Synthetic peptide
AASYRLRITVVVTPDEYHY

SEQ ID NO: 363 - protein, 14 aa, Artificial Sequence
  Description of Artificial Sequence: Synthetic peptide
VKLVDSGWYSAYDY

SEQ ID NO: 364 - protein, 14 aa, Artificial Sequence
  Description of Artificial Sequence: Synthetic peptide
AAQDWATEGYEYDY

SEQ ID NO: 365 - protein, 8 aa, Artificial Sequence
  Description of Artificial Sequence: Synthetic peptide
HFLWGRHY

SEQ ID NO: 366 - protein, 20 aa, Artificial Sequence
  Description of Artificial Sequence: Synthetic peptide
GPSLRTFHGREWYRPPWFTS

SEQ ID NO: 367 - protein, 14 aa, Artificial Sequence
  Description of Artificial Sequence: Synthetic peptide
NARIRGNYGSRIDY

SEQ ID NO: 368 - protein, 8 aa, Artificial Sequence
  Description of Artificial Sequence: Synthetic peptide
ARDYNFES

SEQ ID NO: 369 - protein, 10 aa, Artificial Sequence
  Description of Artificial Sequence: Synthetic peptide
KADTRWGGMY

SEQ ID NO: 370 - protein, 17 aa, Artificial Sequence
  Description of Artificial Sequence: Synthetic peptide
ASRRGVDLRRNSYEYDY

SEQ ID NO: 371 - protein, 11 aa, Artificial Sequence
  Description of Artificial Sequence: Synthetic peptide
GSIFAINEINL

SEQ ID NO: 372 - protein, 8 aa, Artificial Sequence
  Description of Artificial Sequence: Synthetic peptide
GDTRSINL

SEQ ID NO: 373 - protein, 8 aa, Artificial Sequence
  Description of Artificial Sequence: Synthetic peptide
GSIRSINV

SEQ ID NO: 374 - protein, 8 aa, Artificial Sequence
  Description of Artificial Sequence: Synthetic peptide
GFTFNSYA

SEQ ID NO: 375 - protein, 8 aa, Artificial Sequence
  Description of Artificial Sequence: Synthetic peptide
GLTFSNYA

SEQ ID NO: 376 - protein, 8 aa, Artificial Sequence
  Description of Artificial Sequence: Synthetic peptide
GFAFDDYA

SEQ ID NO: 377 - protein, 8 aa, Artificial Sequence
  Description of Artificial Sequence: Synthetic peptide
GATSNVNA

SEQ ID NO: 378 - protein, 8 aa, Artificial Sequence
  Description of Artificial Sequence: Synthetic peptide
GTMFSGKD

SEQ ID NO: 379 - protein, 8 aa, Artificial Sequence
  Description of Artificial Sequence: Synthetic peptide
GNDISGSA

SEQ ID NO: 380 - protein, 8 aa, Artificial Sequence
  Description of Artificial Sequence: Synthetic peptide
GSIFSINA

SEQ ID NO: 381 - protein, 8 aa, Artificial Sequence
  Description of Artificial Sequence: Synthetic peptide
GNMLDLNT

SEQ ID NO: 382 - protein, 7 aa, Artificial Sequence
  Description of Artificial Sequence: Synthetic peptide
CASDGNT

SEQ ID NO: 383 - protein, 8 aa, Artificial Sequence
  Description of Artificial Sequence: Synthetic peptide
INSGGGST

SEQ ID NO: 384 - protein, 8 aa, Artificial Sequence
  Description of Artificial Sequence: Synthetic peptide
INWSGGTT

SEQ ID NO: 385 - protein, 8 aa, Artificial Sequence
  Description of Artificial Sequence: Synthetic peptide
ISWNGGGT

SEQ ID NO: 386 - protein, 7 aa, Artificial Sequence
  Description of Artificial Sequence: Synthetic peptide
ISSGGST

SEQ ID NO: 387 - protein, 7 aa, Artificial Sequence
  Description of Artificial Sequence: Synthetic peptide
VSSDGGT

SEQ ID NO: 388 - protein, 8 aa, Artificial Sequence
  Description of Artificial Sequence: Synthetic peptide
VDAPRERP

SEQ ID NO: 389 - protein, 7 aa, Artificial Sequence
  Description of Artificial Sequence: Synthetic peptide
VSRFGET

SEQ ID NO: 390 - protein, 5 aa, Artificial Sequence
  Description of Artificial Sequence: Synthetic peptide
LGIST